(19) United States
(12) Patent Application Publication
(10) Pub. No.: US 2025/0043319 A1
(43) Pub. Date: Feb. 6, 2025

(54) BIOSYNTHESIS OF CYCLOLAVANDULYL DERIVATIVES OF AROMATIC COMPOUNDS

(71) Applicant: Invizyne Technologies, Inc., Monrovia, CA (US)

(72) Inventors: John BILLINGSLEY, Monrovia, CA (US); Tyler P. KORMAN, Monrovia, CA (US); Mohammad HAYAT, Monrovia, CA (US)

(73) Assignee: Invizyne Technologies, Inc., Monrovia, CA (US)

(21) Appl. No.: 18/918,626

(22) Filed: Oct. 17, 2024

Related U.S. Application Data
(63) Continuation of application No. PCT/US2023/019381, filed on Apr. 21, 2023.
(60) Provisional application No. 63/333,670, filed on Apr. 22, 2022.

(51) Int. Cl. C12P 7/22; C12N 9/10; C12P 17/06

(52) U.S. Cl. CPC C12P 7/22; C12N 9/1085; C12P 17/06; C12Y 205/01084; C12Y 205/01102

(57) ABSTRACT

The present disclosure relates to compositions comprising cyclolavandulyl derivatives of aromatic compounds, such as cannabinoids, flavonoids, and alkaloids, and methods for preparing and using the compositions.

### CROSS REFERENCE TO RELATED APPLICATIONS

This application is a continuation under 35 U.S.C. 120 and 365 (c) of International Application PCT/US2023/019381, filed Apr. 21, 2023, which claims priority benefit of U.S. Provisional Application No. 63/333,670, filed Apr. 22, 2022, each of which is hereby incorporated by reference herein in its entirety for all purposes.

### FIELD

The present disclosure relates to cyclolavandulyl derivatives of aromatic compounds, including cannabinoid, alkaloid, and flavonoid compounds, and compositions and biosynthetic methods for preparing and using these compounds.

### REFERENCE TO SEQUENCE LISTING

The official copy of the Sequence Listing is submitted concurrently with the specification as an WIPO Standard ST.26 formatted XML file with file name “15041-001PV1.xml”, a creation date of Apr. 19, 2023, and a size of 27,126 bytes. This Sequence Listing filed electronically via USPTO EFS-Web is part of the specification and is incorporated in its entirety by reference herein. This sequence listing corresponds to the ST.25 formatted sequence listing with a file name of “15041-001PV1_SeqList_ST25.txt”, a creation date of Feb. 25, 2022, and a size of 44,691 bytes, that was filed with the priority U.S. Provisional Application No. 63/333,670 on Apr. 22, 2022.

### BACKGROUND

Cannabinoids are a large, well-known class of bioactive plant-derived compounds that regulate the cannabinoid receptors (CB1 and CB2) of the human endocannabinoid system. Cannabinoids are promising pharmacological agents with over 100 ongoing clinical trials investigating their therapeutic benefits as antiemetics, anticonvulsants, analgesics, and antidepressants. Further, three cannabinoid therapies have been FDA approved to treat chemotherapy induced nausea, MS spasticity and seizures associated with severe epilepsy.

Although the plant, Cannabis sativa, is known to make over 100 different cannabinoid compounds, the best known and most studied cannabinoids include tetrahydrocannabidiolic acid (THCA), tetrahydrocannabidivarinic acid (THCVA), cannabidiolic acid (CBDA), cannabidivarinic acid (CBDVA), and their decarboxylated analogs (e.g., THC, THCV, CBD, CBDV). Of the cannabinoids made by the plant, all are formed via the enzymatic prenylation with geranyl pyrophosphate (GPP) of the aromatic polyketide substrates, such as olivetolic acid (OA) or divarinic acid (DA). The enzymatic prenylation is carried out by a membrane protein GPP: OA transferase (GOT), also referred to as prenyltransferase. The naturally occurring prenyltransferases found in C. sativa (e.g., PT4, UniProt: A0A455ZJC3) are membrane bound proteins.

A soluble prenyltransferase, NphB (UniProt: A0A2Z4JFA9), has been isolated from Streptomyces sp. CL190. See e.g., U.S. Pat. No. 7,361,483B2. NphB has been further engineered to provide soluble prenyltransferase variants capable of prenylating the aromatic polyketides, OA, or DA with GPP to form the cannabinoid compounds, CBGA or CBGVA, respectively, under a range of biosynthetic conditions. See e.g., WO2019173770A1; WO2019183152A1; WO2020028722A1, WO2021134024A1. The engineered NphB variants can be used in cell-free biosynthesis systems and methods for the preparation of cannabinoid compounds. See e.g., WO2020028722A1 and WO2021134024A1.

NphB has also been shown capable of transferring a range of alkyl groups from pyrophosphate donor substrates (other than GPP) to an aromatic substrate, such as 1,6-dihydroxynaphthalene. See e.g., Johnson et al., “Acceptor substrate determines donor specificity of an aromatic prenyltransferase: expanding the biocatalytic potential of NphB,” Appl. Microbiol. Biotechnol. (2020) 104:4383-4395.

Flavonoids are another large, well-known class of bioactive plant-derived aromatic compounds that exhibit biological activity. Furthermore, prenylated derivatives of flavonoid compounds exhibit enhanced bioactivity. See e.g., Mukai, “Prenylation enhances the biological activity of dietary flavonoids by altering their bioavailability,” Bioscience, Biotechnology and Biochemistry (2018) 82 (2); 207-215.

Cyclolavandulyl is a branched and cyclized carbon C10 monoterpene chemical moiety illustrated in its “forward” configuration by the chemical structure below:

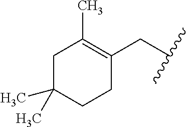 [SMILES: *CC1=C(C)CC(C)(C)CC1]

The biosynthesis of the cyclolavandulyl group occurs in several natural product compounds (e.g., Kallistein A, Seselinonol, Lavanduquinocin, and Lavanducyanin) via an enzymatically catalyzed “non-head-to-tail” condensation and cyclization of two molecules of the C5 dimethylallyl pyrophosphate (DMAPP). See e.g., Ozaki et al., “Cyclolavandulyl Skeleton Biosynthesis via Both Condensation and Cyclization Catalyzed by an Unprecedented Member of the cis-Isoprenyl Diphosphate Synthase Superfamily,” J. Am. Chem. Soc. 2014, 136, 4837-4840. The structure of a cyclolavandulyl diphosphate synthase (CLDS) from Streptomyces spp. CL190 has been structurally characterized and shown to catalyze the conversion of two molecules dimethylallyl pyrophosphate (DMAPP) to the product compound, cyclolavandulyl pyrophosphate. See e.g., Tomita et al., “Structure and mechanism of the monoterpene cyclolavandulyl diphosphate synthase that catalyses consecutive condensation and cyclisation,” Angew. Chemie. Int'l Ed. 2017, 10.1002/anie201708474.

There remains a need for new cannabinoid derivatives, flavonoid derivatives, alkaloid derivatives, and other aromatic derivative compounds, and methods for efficiently producing such compounds.

### SUMMARY

The present disclosure relates generally to cyclolavandulyl derivatives of aromatic compounds, including cannabinoids, alkaloids, and flavonoids, and in vitro biosynthetic methods and compositions for preparing such compounds. This summary is intended to introduce the subject matter of the present disclosure, but does not cover each and every embodiment, combination, or variation that is contemplated and described within the present disclosure. Further embodiments are contemplated and described by the disclosure of the detailed description, drawings, and claims.

In at least one embodiment, the present disclosure provides a compound of structural formula (Ia) or (Ib):

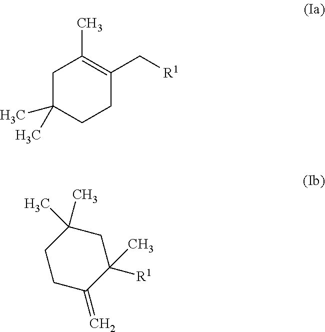

wherein, R¹ is selected from

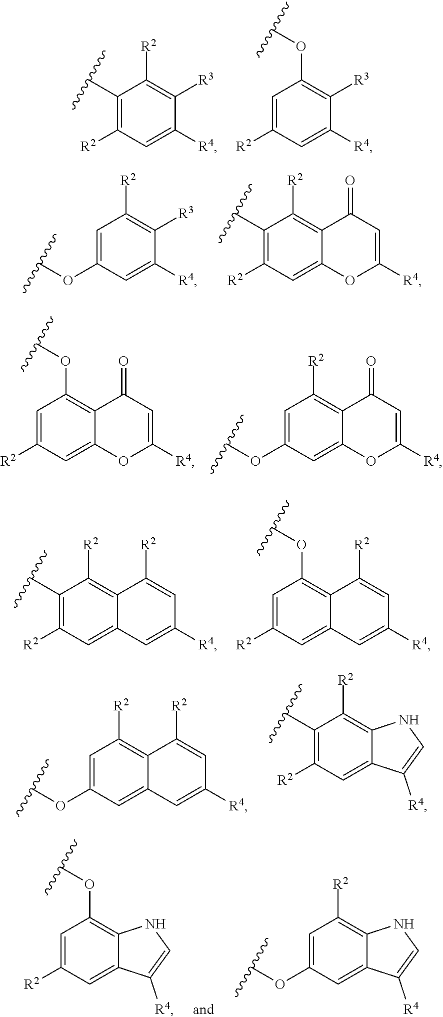

wherein, R² is —H or —OH; R³ is —H or —COOH; and R⁴ is —H, —OH, linear or branched C1-C10 alkyl, linear or branched C1-C10 alkyl-amide, linear or branched C1-C10 alkyl-amine, linear or branched C1-C10 alkyl-alkylene, linear or branched C1-C10 alkyl-alkoxy, or C1-C10 alkyl-aryl, wherein any of the C1-C10 groups is optionally substituted with —OH, —OCH₃, or halogen.

In at least one embodiment of the compound of the present disclosure, R¹ is selected from

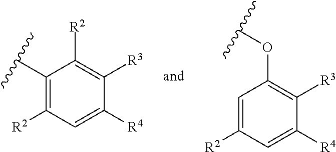

wherein, R² is —H or —OH; R³ is —H or —COOH; and R⁴ is linear or branched C1-C10 alkyl, linear or branched C1-C10 alkyl-amide, linear or branched C1-C10 alkyl-amine, linear or branched C1-C10 alkyl-alkylene, linear or branched C1-C10 alkyl-alkoxy, or C1-C10 alkyl-aryl, wherein any of the C1-C10 groups is optionally substituted with —OH, —OCH₃, or halogen. In at least one embodiment of the compound R² is —OH, and R³ is —COOH. In at least one embodiment of the compound, R⁴ is selected from CH₃, CH₂CH₃, (CH₂)₂CH₃, (CH₂)₃CH₃, (CH₂)₄CH₃, (CH₂)₅CH₃, and (CH₂)₆CH₃; optionally, wherein R² is selected from (CH₂)₂CH₃, (CH₂)₄CH₃, and (CH₂)₆CH₃.

In at least one embodiment of the compound of the present disclosure, the compound is selected from structural formula (IIa), (IIb), (IIc), or (IId):

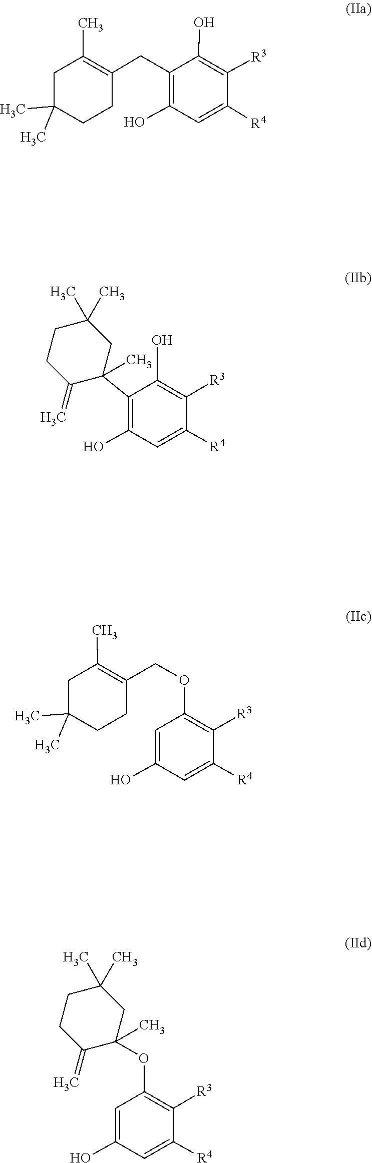

wherein, R³ is —H or —COOH; and R⁴ is linear or branched C1-C10 alkyl, linear or branched C1-C10 alkyl-amide, linear or branched C1-C10 alkyl-amine, linear or branched C1-C10 alkyl-alkylene, linear or branched C1-C10 alkyl-alkoxy, or C1-C10 alkyl-aryl, wherein any of the C1-C10 groups is optionally substituted with —OH, —OCH₃, or halogen. In at least embodiment, R⁴ is selected from CH₃, CH₂CH₃, (CH₂)₂CH₃, (CH₂)₃CH₃, (CH₂)₄CH₃, (CH₂)₅CH₃, (CH₂)₆CH₃, and (CH₂)₇CH₃; optionally, wherein R⁴ is selected from (CH₂)₂CH₃, (CH₂)₄CH₃, and (CH₂)₆CH₃.

In at least one embodiment of the compound of the present disclosure, the compound is selected from compounds (2a), (2b), (2c), (2d), (2e), (2f), (2g), (2h), (2i), (2j), (2k), (2l), (2m), (2n), (2o), (2p), (2q), (2r), (2s), (2t), (2u), (2v), (2w), (2x), (2y), (2z), (2aa), (2bb), (2cc), (2dd), (2ee), (2ff), (2gg), (2hh), (2ii), (2jj), (2kk), (2ll), (2 mm), (2nn), (2oo), (2pp), (2qq), (2rr), (2ss), (2tt), (2uu), (2vv), (2ww), and (2xx):

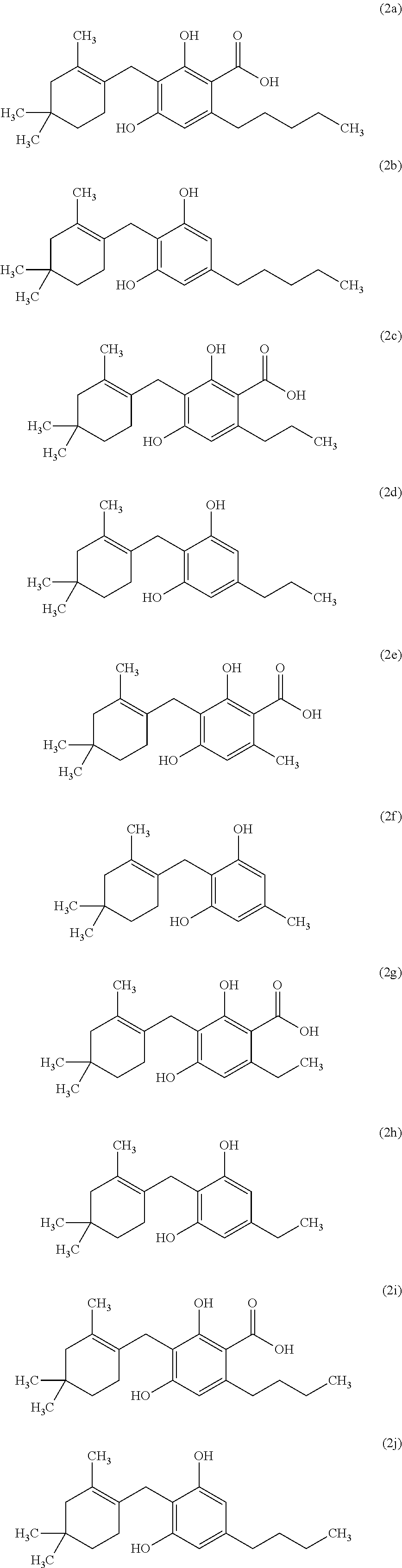

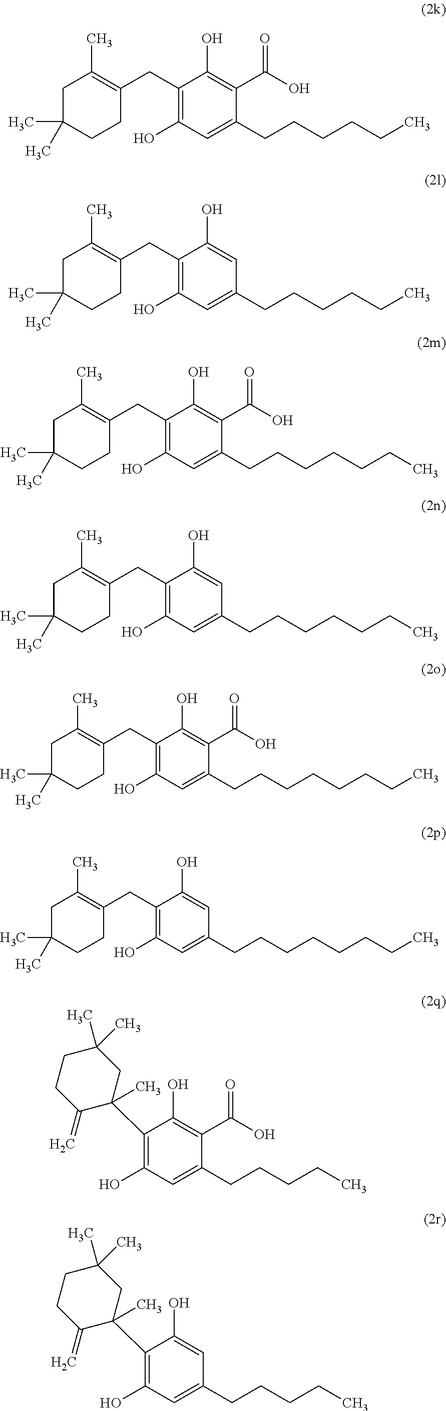

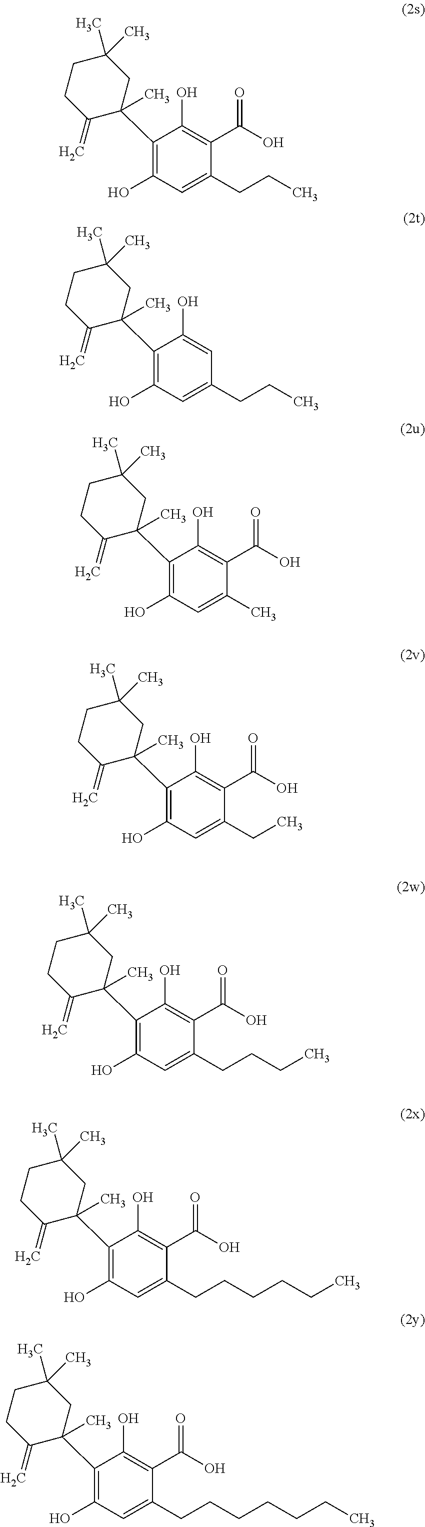

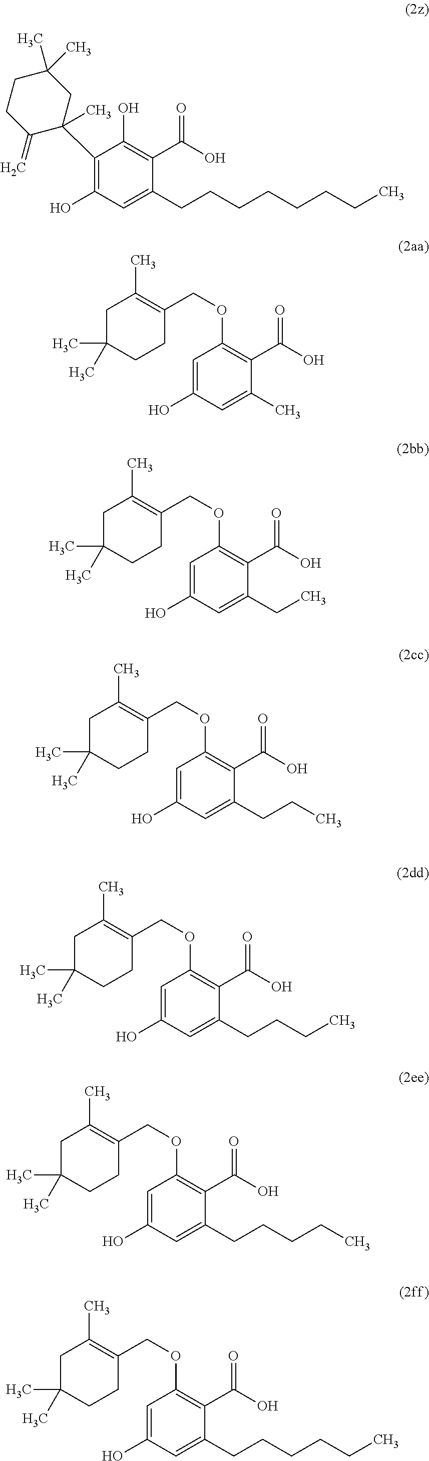

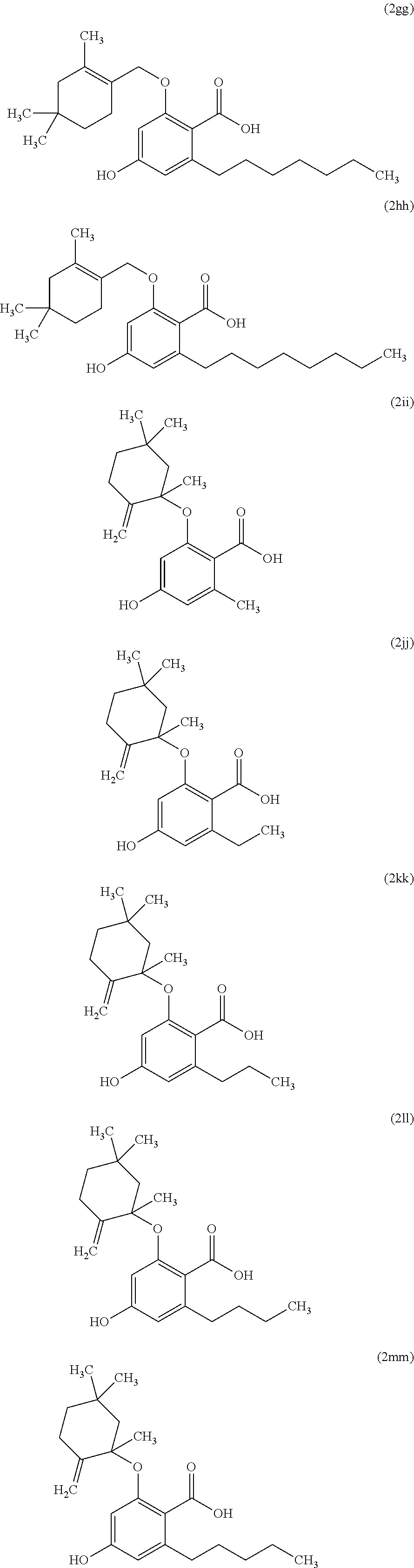

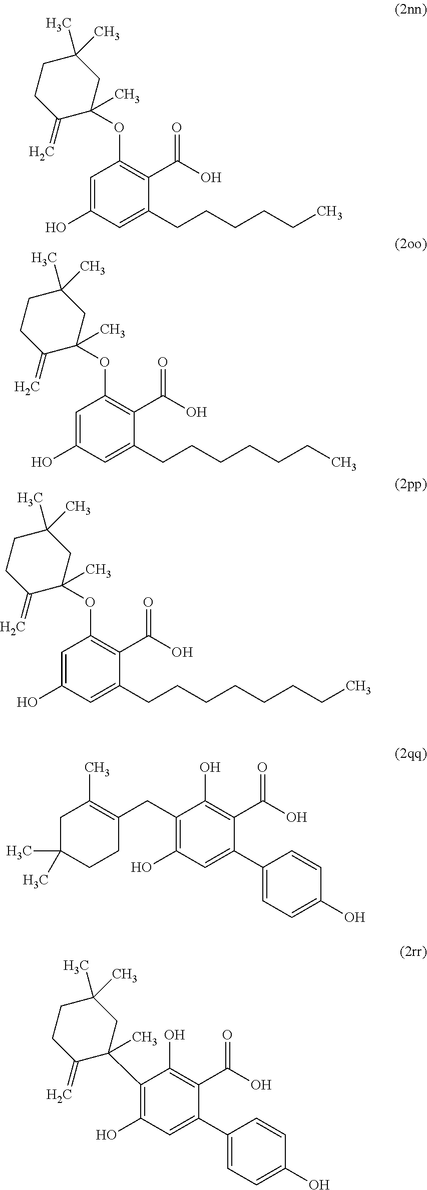

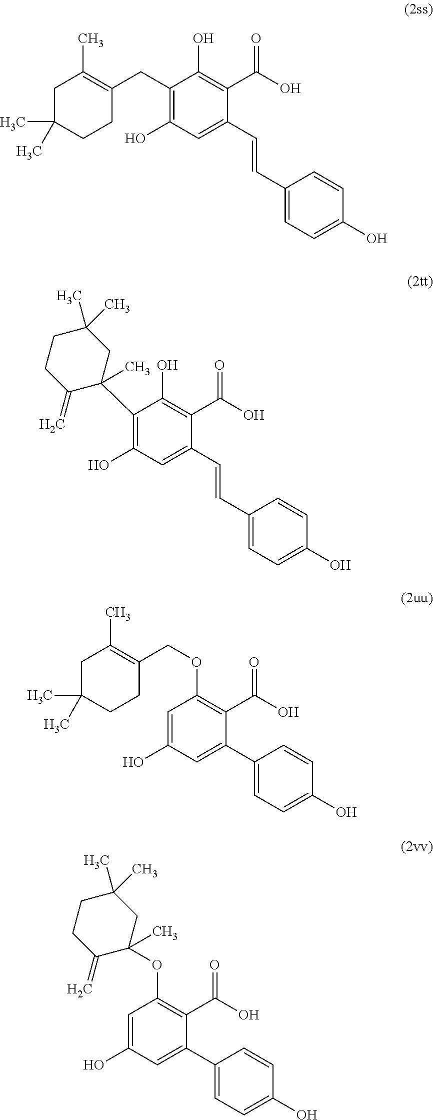 [SMILES: CC1=C(Cc2c(O)cc(/C=C/c3ccc(O)cc3)c(C(=O)O)c2O)CCC(C)(C)C1 ; C=C1CCC(C)(C)CC1(C)c1c(O)cc(/C=C/c2ccc(O)cc2)c(C(=O)O)c1O ; CC1=C(COc2cc(O)cc(-c3ccc(O)cc3)c2C(=O)O)CCC(C)(C)C1 ; C=C1CCC(C)(C)CC1(C)Oc1cc(O)cc(-c2ccc(O)cc2)c1C(=O)O]

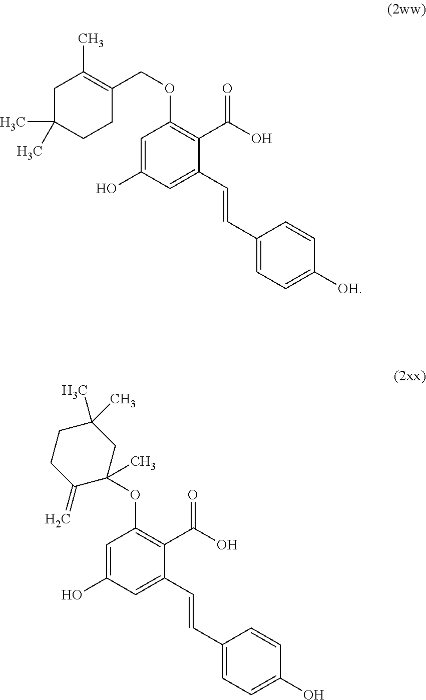 [SMILES: CC1=C(COc2cc(O)cc(/C=C/c3ccc(O)cc3)c2C(=O)O)CCC(C)(C)C1 ; C=C1CCC(C)(C)CC1(C)Oc1cc(O)cc(/C=C/c2ccc(O)cc2)c1C(=O)O]

In at least one embodiment of the compound of the present disclosure, R¹ is selected from:

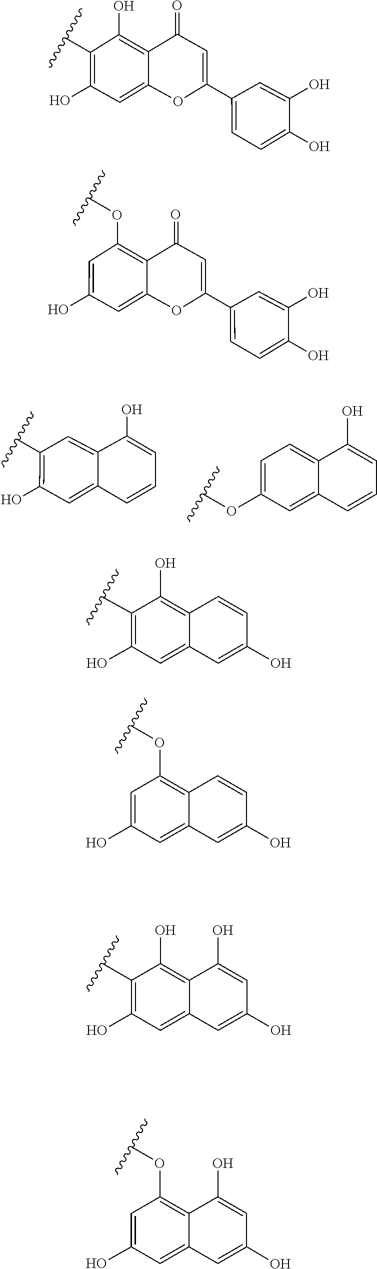

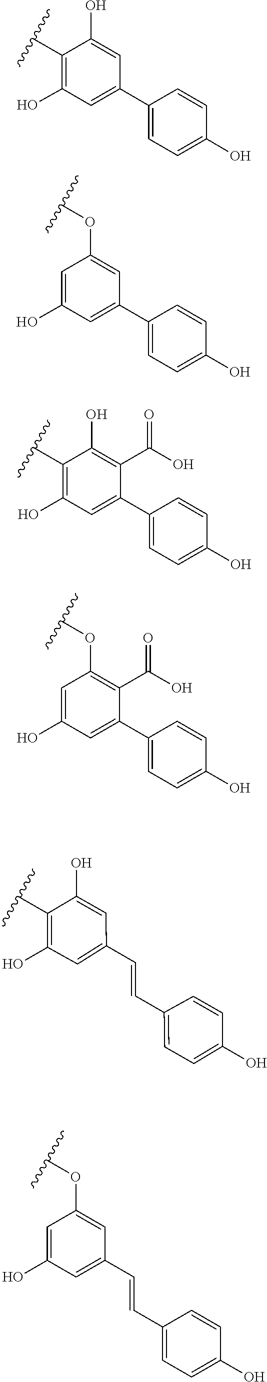 [SMILES: *c1c(O)cc(-c2ccc(O)cc2)cc1O ; *Oc1cc(O)cc(-c2ccc(O)cc2)c1 ; *c1c(O)cc(-c2ccc(O)cc2)c(C(=O)O)c1O ; *Oc1cc(O)cc(-c2ccc(O)cc2)c1C(=O)O ; *c1c(O)cc(/C=C/c2ccc(O)cc2)cc1O ; *Oc1cc(O)cc(/C=C/c2ccc(O)cc2)c1]

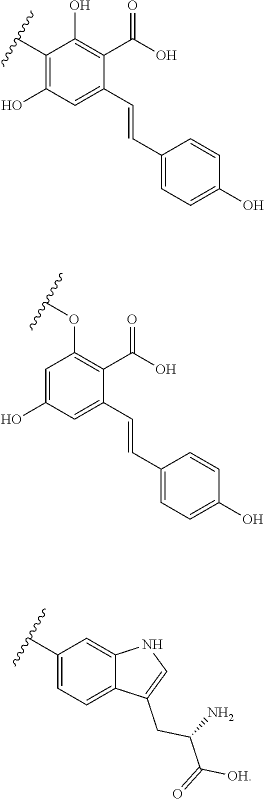 [SMILES: *c1c(O)cc(/C=C/c2ccc(O)cc2)c(C(=O)O)c1O ; *Oc1cc(O)cc(/C=C/c2ccc(O)cc2)c1C(=O)O ; *c1ccc2c(C[C@H](N)C(=O)O)c[nH]c2c1]

In at least one embodiment, the present disclosure provides a method for preparing a cyclolavandulyl-substituted aromatic compound comprising: (a) contacting under suitable reaction conditions: a prenyitransferase, a cyclolavandulyl pyrophosphate (CLPP) of compound (1)

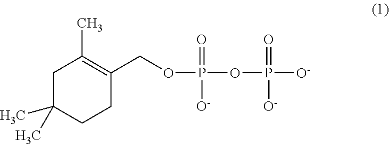 [SMILES: CC1=C(COP(=O)([O-])OP(=O)([O-])[O-])CCC(C)(C)C1]

and an aromatic compound selected from a compound of structural formulas (II), (IV), (V), and

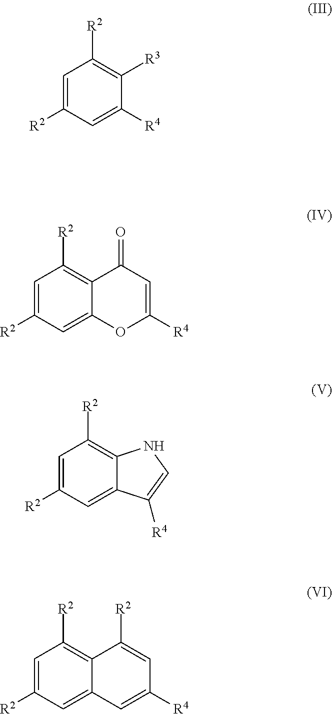

wherein, R² is —H or —OH, R³ is —H or —COOH; and R⁴ is —H, —OH, linear or branched C1-C10 alkyl, linear or branched C1-C10 alkyl-amide, linear or branched C1-C10 alkyl-amine, linear or branched C1-C10 alkyl-alkylene, linear or branched C1-C10 alkyl-alkoxy, or C1-C10 alkyl-aryl, wherein any of the C1-C10 groups is optionally substituted with a —OH, —OCH₃, or a halogen; and (b) recovering the cyclolavandulyl-substituted aromatic compound from the reaction mixture.

In another embodiment, the present disclosure provides a method for preparing a cyclolavandulyl-substituted aromatic compound comprising: (a) contacting under suitable reaction conditions: cyclolavandulyl diphosphate synthase (CLDS), dimethylallyl pyrophosphate, a prenyltransferase, and an aromatic compound selected from a compound of structural formulas (III), (IV), (V), and (VI), wherein, R² is —H or —OH, R³ is —H or —COOH; and R⁴ is —H, —OH, linear or branched C1-C10 alkyl, linear or branched C1-C10 alkyl-amide, linear or branched C1-C10 alkyl-amine, linear or branched C1-C10 alkyl-alkylene, or linear or branched C1-C10 alkyl-alkoxy, wherein any of the linear or branched C1-C10 chains is optionally substituted with a halogen; and (b) recovering the cyclolavandulyl-substituted aromatic compound from the reaction mixture. In at least one embodiment of the method, the cyclolavandulyl diphosphate synthase (CLDS) is a polypeptide comprising an amino acid sequence of SEQ ID NO: 2 or 4, or a variant of SEQ ID NO: 2 or 4; optionally, wherein the variant of SEQ ID NO: 2 comprises an amino acid sequence having at least 90%, at least 95%, at least 97%, at least 98%, or at least 99% identity to SEQ ID NO: 2 or 4.

In at least one embodiment of any of the methods for preparing a cyclolavandulyl-substituted aromatic compound of the present disclosure, the prenyltransferase is selected from: (i) NphB (SEQ ID NO: 8) or a variant of NphB comprising an amino acid sequence having at least 90%, at least 95%, at least 97%, at least 98%, or at least 99% identity to any one of SEQ ID NO: 8; (ii) NphBM31s (SEQ ID NO: 6) or a variant of NphBM31s comprising an amino acid sequence having at least 90%, at least 95%, at least 97%, at least 98%, or at least 99% identity to any one of SEQ ID NO: 6; (iii) a prenyltransferase comprising an amino acid sequence or any one of SEQ ID NO: 9, 10, 11, 12, 13, 14, 15, 16, 17, 18, or 19; and (iv) a variant prenyltransferase comprising an amino acid sequence having at least 90%, at least 95%, at least 97%, at least 98%, or at least 99% identity to any one of SEQ ID NO: 9, 10, 11, 12, 13, 14, 15, 16, 17, 18, or 19.

In at least one embodiment of the methods for preparing a cyclolavandulyl-substituted aromatic compound, the cyclolavandulyl-substituted aromatic compound is selected from compound (4a), (4b), (5a), (5b), (6a), (6b), (7a), (7b), (8a), and (8b):

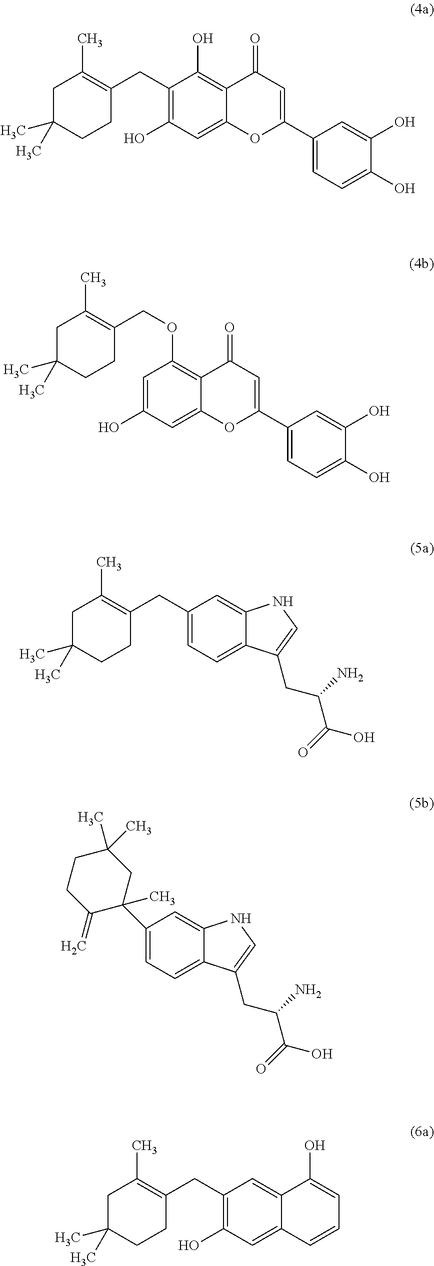 [SMILES: CC1=C(Cc2c(O)cc3oc(-c4ccc(O)c(O)c4)cc(=O)c3c2O)CCC(C)(C)C1 ; CC1=C(COc2cc(O)cc3oc(-c4ccc(O)c(O)c4)cc(=O)c23)CCC(C)(C)C1 ; CC1=C(Cc2ccc3c(C[C@H](N)C(=O)O)c[nH]c3c2)CCC(C)(C)C1 ; C=C1CCC(C)(C)CC1(C)c1ccc2c(C[C@H](N)C(=O)O)c[nH]c2c1 ; CC1=C(Cc2cc3c(O)cccc3cc2O)CCC(C)(C)C1]

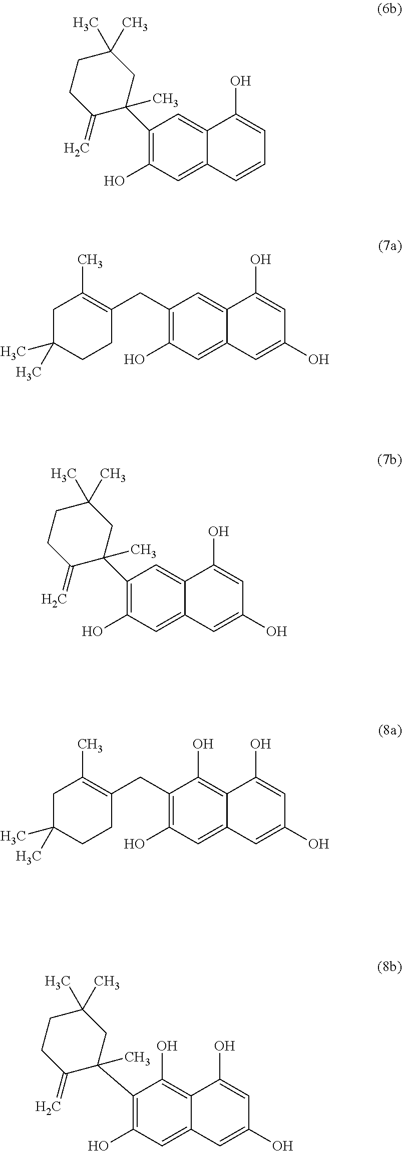 [SMILES: C=C1CCC(C)(C)CC1(C)c1cc2c(O)cccc2cc1O ; CC1=C(Cc2cc3c(O)cc(O)cc3cc2O)CCC(C)(C)C1 ; C=C1CCC(C)(C)CC1(C)c1cc2c(O)cc(O)cc2cc1O ; CC1=C(Cc2c(O)cc3cc(O)cc(O)c3c2O)CCC(C)(C)C1 ; C=C1CCC(C)(C)CC1(C)c1c(O)cc2cc(O)cc(O)c2c1O]

In at least one embodiment of the methods for preparing a cyclolavandulyl-substituted aromatic compound, the aromatic compound is a compound of structural formula (I), wherein, R² is —H or —OH; R³ is —H or —COOH; and R′ is linear or branched C1-C10 alkyl, linear or branched C1-C10 alky-amide, linear or branched C1-C10 alky-amine, linear or branched C1-C10 alkyl-alkylene, linear or branched C1-C10 alkyl-alkoxy, or C1-C10 alkyl-aryl, wherein any of the C1-C10 groups is optionally substituted with a —OH, —OCH₃, or a halogen. In at least one embodiment, R² is —OH, and R³ is —COOH. In at least one embodiment, R⁴ is selected from CH₃, CH₂CH₃, (CH₂)₂CH₃, (CH₂)₃CH₃, (CH₂)₄CH₃, (CH₂)₅CH₃, (CH₂)₆CH₃, and (CH₂)₇CH₃; optionally, wherein R⁴ is selected from (CH₂)₂CH₃, (CH₂)₄CH₃, and (CH₂)₆CH₃. In at least one embodiment, the aromatic compound is a cannabinoid precursor compound selected from divarinic acid (DA), olivetolic acid (OA), and phorolic acid (PA).

In at least one embodiment of the methods for preparing a cyclolavandulyl-substituted aromatic compound, the aromatic compound of structural formula (III) is selected from any one of compounds (3a), (3b), (3c), (3d), (3e), (3f), (3g), and (3h):

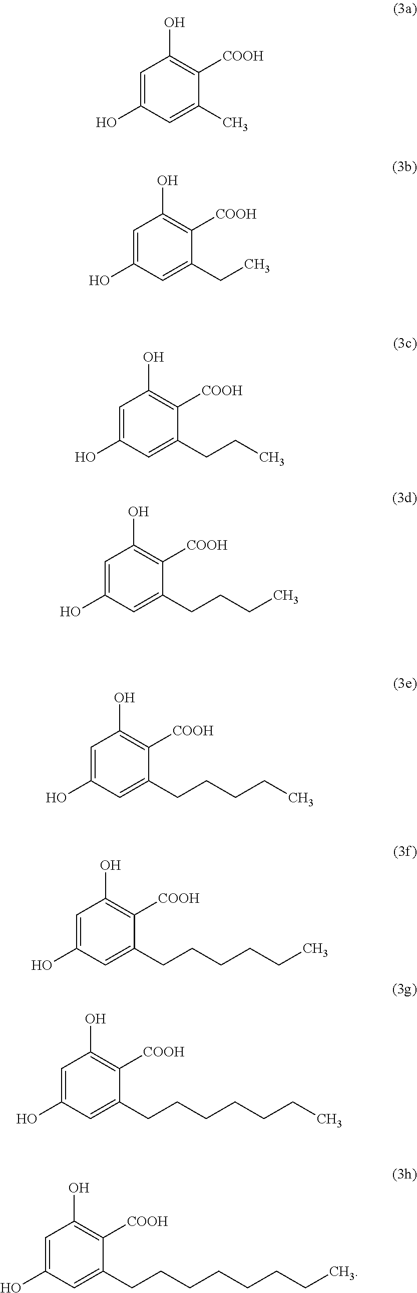

In at least one embodiment of the methods for preparing a cyclolavandulyl-substituted aromatic compound, the recovered cyclolavandulyl-substituted aromatic compound has a structural formula (IIa), (IIb), (IIc), or (IId):

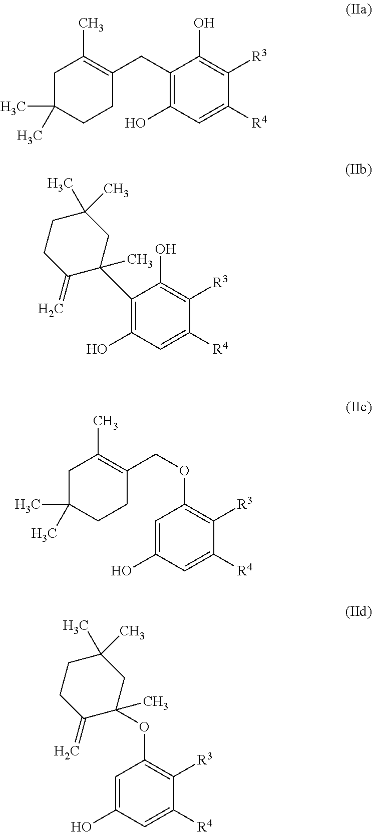

wherein, R³ is —H or —COOH; and R⁴ is R⁴ is linear or branched C1-C10 alkyl, linear or branched C1-C10 alkyl-amide, linear or branched C1-C10 alkyl-amine, linear or branched C1-C10 alkyl-alkylene, linear or branched C1-C10 alkyl-alkoxy, or C1-C10 alkyl-aryl, wherein any of the C1-C10 groups is optionally substituted with a —OH, —OCH₃, or a halogen.

In at least one embodiment of the method, the recovered cyclolavandulyl-substituted compound is a cyclolavandulyl-substituted cannabinoid; optionally, wherein the cyclolavandulyl-substituted cannabinoid is selected from compounds (2a), (2b), (2c), (2d), (2e), (2f), (2g), (2h), (2i), (2j), (2k), (2l), (2m), (2n), (2o), (2p), (2q), (2r), (2s), (2t), (2u), (2v), (2w), (2x), (2y), (2z), (2aa), (2bb), (2cc), (2dd), (2ee), (2ff), (2gg), (2hh), (2ii), (2jj), (2kk), (2ll), (2 mm), (2nn), (2oo), and (2pp).

In at least one embodiment of any of the methods for preparing a cyclolavandulyl-substituted cannabinoid compounds of the present disclosure, the method can further comprise a step of decarboxylating the recovered cyclolavandulyl-substituted compound. For example, the carboxylated cyclolavandulyl-substituted cannabinoid of compound (2a) can be decarboxylated to produce a compound of cyclolavandulyl-substituted cannabinoid of compound (2b).

In another embodiment, the present disclosure also provides compositions of reactants used in the methods. Accordingly, in at least one embodiment the disclosure provides a composition comprising a prenyltransferase, a cyclolavandulyl pyrophosphate of compound (1)

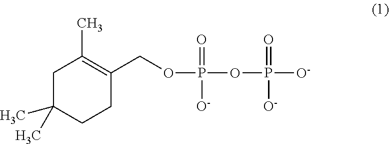 [SMILES: CC1=C(COP(=O)([O-])OP(=O)([O-])[O-])CCC(C)(C)C1]

and an aromatic compound selected from a compound of structural formulas (III), (IV), (V), and (VI), wherein, R² is —H or —OH, R³ is —H or —COOH; and R⁴ is —H, —OH, linear or branched C1-C10 alkyl, linear or branched C1-C10 alkyl-amide, linear or branched C1-C10 alkyl-amine, linear or branched C1-C10 alkyl-alkylene, linear or branched C1-C10 alkyl-alkoxy, or C1-C10 alkyl-aryl, wherein any of the C1-C10 groups is optionally substituted with a —OH, —OCH₃, or a halogen.

In at least one embodiment, the present disclosure provides a composition comprising a cyclolavandulyl diphosphate synthase (CLDS), dimethylallyl pyrophosphate, a prenyltransferase, and an aromatic compound selected from a compound of structural formulas (III), (IV), (V), and (VI), wherein, R² is —H or —OH, R³ is —H or —COOH; and R⁴ is —H, —OH, linear or branched C1-C10 alkyl, linear or branched C1-C10 alkyl-amide, linear or branched C1-C10 alkyl-amine, linear or branched C1-C10 alkyl-alkylene, linear or branched C1-C10 alkyl-alkoxy, or C1-C10 alkyl-aryl, wherein any of the C1-C10 groups is optionally substituted with a —OH, —OCH₃, or a halogen. In at least one embodiment, the cyclolavandulyl diphosphate synthase (CLDS) is a polypeptide comprising an amino acid sequence of SEQ ID NO: 2 or 4, or a variant of SEQ ID NO: 2 or 4; optionally, wherein the variant of SEQ ID NO: 2 or 4 comprises an amino acid sequence having at least 90%, at least 95%, at least 97%, at least 98%, or at least 99% identity to SEQ ID NO: 2 or 4.

In at least one embodiment of the compositions, the prenyltransferase is selected from: (i) NphB (SEQ ID NO: 8) or a variant of NphB comprising an amino acid sequence having at least 90%, at least 95%, at least 97%, at least 98%, or at least 99% identity to any one of SEQ ID NO: 8; (ii) NphBM31s (SEQ ID NO: 6) or a variant of NphBM31s comprising an amino acid sequence having at least 90%, at least 95%, at least 97%, at least 98%, or at least 99% identity to any one of SEQ ID NO: 6; (iii) a prenyltransferase comprising an amino acid sequence or any one of SEQ ID NO: 9, 10, 11, 12, 13, 14, 15, 16, 17, 18, or 19; and (iv) a variant prenyltransferase comprising an amino acid sequence having at least 90%, at least 95%, at least 97%, at least 98%, or at least 99% identity to any one of SEQ ID NO: 9, 10, 11, 12, 13, 14, 15, 16, 17, 18, or 19.

In at least one embodiment of the compositions, the aromatic compound is a compound of structural formula (III), wherein, R² is —H or —OH; R³ is —H or —COOH; and R⁴ is —H, —OH, linear or branched C1-C10 alkyl, linear or branched C1-C10 alkyl-amide, linear or branched C1-C10 alkyl-amine, linear or branched C1-C10 alkyl-alkylene, linear or branched C1-C10 alkyl-alkoxy, or C1-C10 alkyl-aryl, wherein any of the C1-C10 groups is optionally substituted with a —OH, —OCH₃, or a halogen. In at least one embodiment, R⁴ is selected from CH₃, CH₂CH₃, (CH₂)₂CH₃, (CH₂)₃CH₃, (CH₂)₄CH₃, (CH₂)₅CH₃, (CH₂)₆CH₃, and (CH₂)₇CH₃; optionally, wherein R⁴ is selected from (CH₂)₂CH₃, (CH₂)₄CH₃, and (CH₂)₆CH₃. In at least one embodiment, the aromatic compound is a cannabinoid precursor compound selected from divarinic acid (DA), olivetolic acid (OA), and phorolic acid (PA).

In at least one embodiment of the compositions, the aromatic compound of structural formula (III) is selected from any one of compounds (3a), (3b), (3c), (3d), (3e), (3f), (3g), and (3h).

In at least one embodiment of the compositions, the composition further comprises a cyclolavandulyl-substituted aromatic compound is selected from compound (3i), (3j), (3k), (31), (4a), (5a), (6a), and (6b). In at least one embodiment, the cyclolavandulyl-substituted compound is a cyclolavandulyl-substituted cannabinoid; optionally, wherein the cyclolavandulyl-substituted cannabinoid is selected from compounds (2a), (2b), (2c), (2d), (2e), (2f), (2g), (2h), (2i), (2j), (2k), (2l), (2m), (2n), (2o), (2p), (2q), (2r), (2s), (2t), (2u), (2v), (2w), (2x), (2y), (2z), (2aa), (2bb), (2cc), (2dd), (2ee), (2ff), (2gg), (2hh), (2ii), (2jj), (2kk), (2ll), (2 mm), (2nn), (2oo), (2pp), (2qq), (2rr), (2ss), (2tt), (2uu), (2vv), (2ww), and (2xx).

### BRIEF DESCRIPTION OF THE DRAWINGS

A better understanding of the novel features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 depicts a schematic overview of steps, molecular inputs/outputs, and enzymes involved in cannabinoid biosynthesis relevant to the biosynthetic methods compositions for preparing the cyclolavandulyl derivatives of cannabinoids of the present disclosure.

FIG. 2 depicts exemplary prenylation reactions producing the standard cannabinoids, CBGVA, CBGA, and CBGPA, and the alternative “cyclolavandulylation” reactions using cyclolavandulyl pyrophosphate (CLPP) as the cyclolavandulyl group donor to an acceptor cannabinoid precursor substrate to produce the cyclolavandulyl-cannabinoid compounds, CBCLVA, and CBCLA.

FIG. 3 depicts LC-MS results of the in vitro biosynthesis of cyclolavandulyl-CBCLA, as described in Example 1.

FIG. 4 depicts exemplary LC-MS results of the in vitro biosynthesis of the standard cannabinoids, CBGVA, CBGA, and CBGPA, and the cyclolavandulylated cannabinoid compounds, CBCLVA, and CBCLA.

FIG. 5A, FIG. 5B, FIG. 5C, FIG. 5D, and FIG. 5E depicts LC-MS results for the cell-free biosynthesis of 5 different cyclolavandulylated aromatic compounds prepared as described in Example 2. FIG. 5A: HPLC results for cyclolavandulyl-resveratrol; FIG. 5B: HPLC results for cyclolavandulyl-luteolin; FIG. 5C: HPLC results for cyclolavandulyl-naringenin; FIG. 5D: HPLC results for cyclolavandulyl-tryptophan; and FIG. 5E: HPLC results for cyclolavandulyl-olivetolic acid.

### DETAILED DESCRIPTION

For the descriptions herein and the appended claims, the singular forms “a”, and “an” include plural referents unless the context clearly indicates otherwise. Thus, for example, reference to “a protein” includes more than one protein, and reference to “a compound” refers to more than one compound. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. The use of “comprise,” “comprises,” “comprising” “include,” “includes,” and “including” are interchangeable and not intended to be limiting. It is to be further understood that where descriptions of various embodiments use the term “comprising,” those skilled in the art would understand that in some specific instances, an embodiment can be alternatively described using language “consisting essentially of” or “consisting of.”

Where a range of values is provided, unless the context clearly dictates otherwise, it is understood that each intervening integer of the value, and each tenth of each intervening integer of the value, unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of these limits, ranges excluding (i) either or (ii) both of those included limits are also included in the invention. For example, “1 to 50,” includes “2 to 25,” “5 to 20,” “25 to 50,” “1 to 10,” etc.

Generally, the nomenclature used herein and the techniques and procedures described herein include those that are well understood and commonly employed by those of ordinary skill in the art, such as the common techniques and methodologies described in e.g., Green and Sambrook, Molecular Cloning: A Laboratory Manual (Fourth Edition), Vols. 1-3, Cold Spring Harbor Laboratory, Cold Spring Harbor, N.Y., 2012 (hereinafter “Sambrook”); and Current Protocols in Molecular Biology, F. M. Ausubel et al., eds., originally published in 1987 in book form by Greene Publishing Associates, Inc. and John Wiley & Sons, Inc., and regularly supplemented through 2011, and now available in journal format online as Current Protocols in Molecular Biology, Vols. 00-130, (1987-2020), published by Wiley & Sons, Inc. in the Wiley Online Library (hereinafter “Ausubel”).

All publications, patents, patent applications, and other documents referenced in this disclosure are hereby incorporated by reference in their entireties for all purposes to the same extent as if each individual publication, patent, patent application or other document were individually indicated to be incorporated by reference herein for all purposes.

Unless defined otherwise, all technical and scientific terms used herein have the same meaning as commonly understood by one of ordinary skill in the art to which the present invention pertains. It is to be understood that the terminology used herein is for describing particular embodiments only and is not intended to be limiting. For purposes of interpreting this disclosure, the following description of terms will apply and, where appropriate, a term used in the singular form will also include the plural form and vice versa.

### Definitions

“Cyclolavandulyl group” refers to a branched and cyclized carbon C10 monoterpene group in either the “forward” or “reverse” form as illustrated by the chemical moiety structures below:

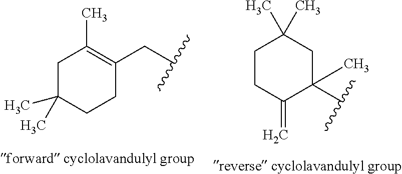 [SMILES: *CC1=C(C)CC(C)(C)CC1 ; *C1(C)CC(C)(C)CCC1=C]

The cyclolavandulyl group is known to occur in several natural product compounds, including Kallistein A, Seselinonol, Lavanduquinocin, and Lavanducyanin. The biosynthesis of cyclolavandulyl structure occurs via the enzymatically catalyzed “non-head-to-tail” condensation and cyclization of two molecules of the C5 dimethylallyl pyrophosphate (DMAPP). See e.g., Ozaki et al., “Cyclolavandulyl Skeleton Biosynthesis via Both Condensation and Cyclization Catalyzed by an Unprecedented Member of the cis-Isoprenyl Diphosphate Synthase Superfamily,” J. Am. Chem. Soc. 2014, 136, 4837-4840.

“Cyclolavandulyl diphosphate synthase” or “CLDS” refers to an enzyme capable of catalyzing the conversion of two molecules dimethylallyl pyrophosphate (DMAPP) to the product compound, cyclolavandulyl pyrophosphate (CLPP) (compound (1)). A “CLDS” polypeptide can include any naturally occurring, recombinant, and/or engineered (variant) polypeptides having the CLDS activity of Scheme 1, and is intended to include enzymes, such as the wild-type CLDS from Streptomyces sp. CL190 (GenBank accession: BAO66170.1; PDB: 5YGJ_A; UniProt entry X5IYJ5), as well as, recombinant or engineered polypeptides derived from the wild-type CL190 enzyme, and any other enzymes having CLDS activity.

“Prenyltransferase” or “aromatic prenyltransferase” or “PT” as used herein, refers to an enzyme capable of catalyzing the transfer of a prenyl pyrophosphate donor substrate (e.g., geranyl pyrophosphate) to an aromatic acceptor compound (e.g., olivetolic acid). An aromatic prenyltransferase (PT) polypeptide can include naturally occurring and recombinant polypeptides having the PT activity of Scheme 2, and is intended to include enzymes, such as the wild-type NphB from Streptomyces sp. CL190, as well as, recombinant or engineered polypeptide variants derived from the wild-type NphB enzyme, such as NphBM31s and others disclosed in WO2021134024A1, which is hereby incorporated by reference herein, and other naturally occurring enzymes having PT activity, including those disclosed in the Examples. The variant NphBM31s has the following mutations relative to the wild-type: M141, Y31W, T69P, T771, T981, S136A, E222D, G224S, A232S, N236T, Y288V and G297K. As disclosed elsewhere herein, it is a surprising discovery of the present disclosure that enzymes with PT activity can also catalyze the transfer of a cyclolavandulyl group from a pyrophosphate donor substrate, such as CLPP (compound (1)) to an aromatic compound.

“Cannabinoid” refers to a compound that acts on cannabinoid receptor and is intended to include the endocannabinoid compounds that are produced naturally in animals, the phytocannabinoid compounds produced naturally in cannabis plants, and the synthetic cannabinoids compounds. Exemplary cannabinoids are provided in Table 1, and include, but are not limited to, cannabigerolic acid (CBGA) and cannabigerovarinic acid (CBGVA).

TABLE 1
Exemplary cannabinoid compounds

| | Abbrev. | |
|---|---|---|
| Compound Name | Name | Chemical Structure |
| cannabigerolic acid | CBGA | 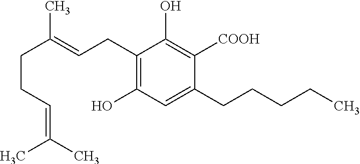 [SMILES: CCCCCc1cc(O)c(C/C=C(\C)CCC=C(C)C)c(O)c1C(=O)O] |
| cannabigerol | CBG | 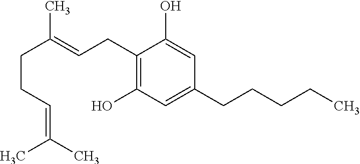 [SMILES: CCCCCc1cc(O)c(C/C=C(\C)CCC=C(C)C)c(O)c1] |
| Δ⁹-tetrahydrocannabinolic acid | Δ⁹-THCA | 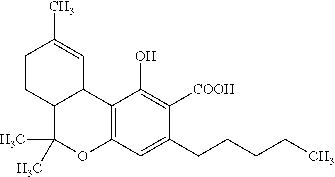 [SMILES: CCCCCc1cc2c(c(O)c1C(=O)O)C1C=C(C)CCC1C(C)(C)O2] |
| Δ⁹-tetrahydrocannabinol | Δ⁹-THC | 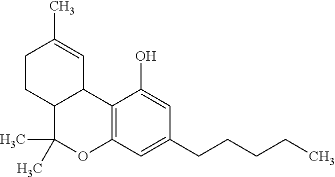 [SMILES: CCCCCc1cc(O)c2c(c1)OC(C)(C)C1CCC(C)=CC21] |
| Δ⁸-tetrahydrocannabinolic acid | Δ⁸-THCA | 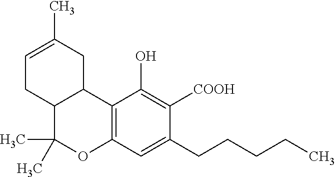 [SMILES: CCCCCc1cc2c(c(O)c1C(=O)O)C1CC(C)=CCC1C(C)(C)O2] |
| Δ⁸-tetrahydrocannabinol | Δ⁸-THC | 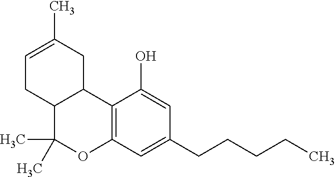 [SMILES: CCCCCc1cc(O)c2c(c1)OC(C)(C)C1CC=C(C)CC21] |
| cannabidiolic acid | CBDA | 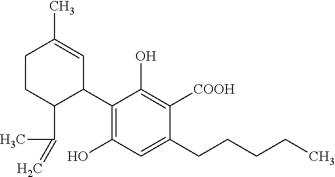 [SMILES: C=C(C)C1CCC(C)=CC1c1c(O)cc(CCCCC)c(C(=O)O)c1O] |
| cannabidiol | CBD | 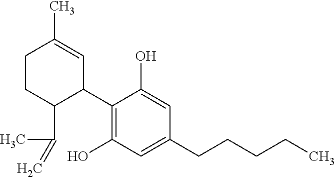 [SMILES: C=C(C)C1CCC(C)=CC1c1c(O)cc(CCCCC)cc1O] |
| cannabichromenic acid | CBCA | 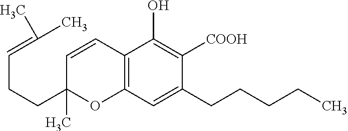 [SMILES: CCCCCc1cc2c(c(O)c1C(=O)O)C=CC(C)(CCC=C(C)C)O2] |
| cannabichromene | CBC | 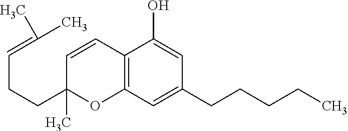 [SMILES: CCCCCc1cc(O)c2c(c1)OC(C)(CCC=C(C)C)C=C2] |
| cannabinolic acid | CBNA | 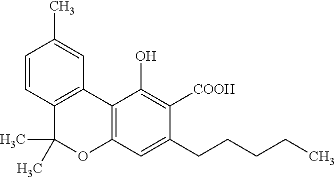 [SMILES: CCCCCc1cc2c(c(O)c1C(=O)O)-c1cc(C)ccc1C(C)(C)O2] |
| cannabinol | CBN | 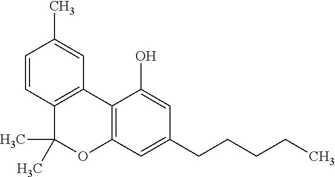 [SMILES: CCCCCc1cc(O)c2c(c1)OC(C)(C)c1ccc(C)cc1-2] |
| cannabidivarinic acid | CBDVA | 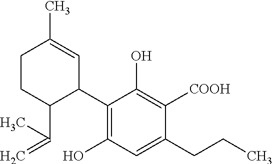 [SMILES: C=C(C)C1CCC(C)=CC1c1c(O)cc(CCC)c(C(=O)O)c1O] |
| cannabidivarin | CBDV | 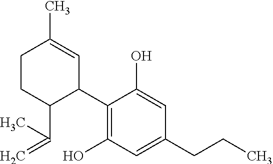 [SMILES: C=C(C)C1CCC(C)=CC1c1c(O)cc(CCC)cc1O] |
| Δ⁹- tetrahydrocannabivarinic acid | Δ⁹- THCVA | 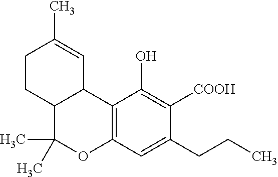 [SMILES: CCCc1cc2c(c(O)c1C(=O)O)C1C=C(C)CCC1C(C)(C)O2] |
| Δ⁹-tetrahydrocannabivarin | Δ⁹-THCV | 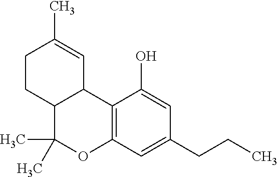 [SMILES: CCCc1cc(O)c2c(c1)OC(C)(C)C1CCC(C)=CC21] |
| Cannabidibutolic acid | CBDBA | 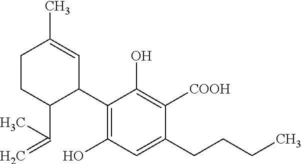 [SMILES: C=C(C)C1CCC(C)=CC1c1c(O)cc(CCCC)c(C(=O)O)c1O] |
| Cannabidibutol | CBDB | 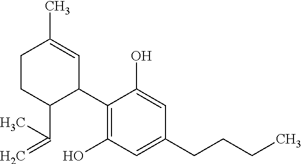 [SMILES: C=C(C)C1CCC(C)=CC1c1c(O)cc(CCCC)cc1O] |
| Δ⁹-tetrahydrocannabutolic acid | Δ⁹- THCBA | 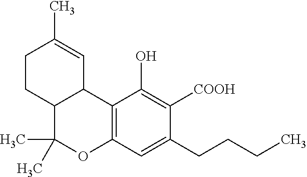 [SMILES: CCCCc1cc2c(c(O)c1C(=O)O)C1C=C(C)CCC1C(C)(C)O2] |
| Δ⁹-tetrahydrocannabutol | Δ⁹-THCB | 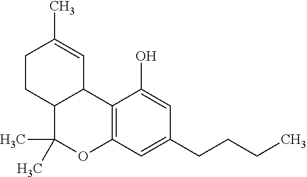 [SMILES: CCCCc1cc(O)c2c(c1)OC(C)(C)C1CCC(C)=CC21] |
| Cannabidiphorolic acid | CBDPA | 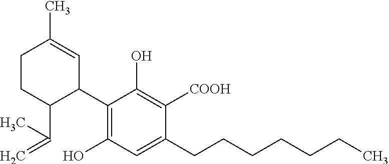 [SMILES: C=C(C)C1CCC(C)=CC1c1c(O)cc(CCCCCCC)c(C(=O)O)c1O] |
| Cannabidiphorol | CBDP | 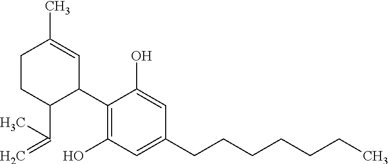 [SMILES: C=C(C)C1CCC(C)=CC1c1c(O)cc(CCCCCCC)cc1O] |
| Δ⁹- tetrahydrocannabiphorolic acid | Δ⁹- THCPA | 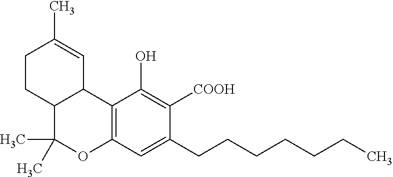 [SMILES: CCCCCCCc1cc2c(c(O)c1C(=O)O)C1C=C(C)CCC1C(C)(C)O2] |
| Δ⁹- tetrahydrocannabiphorol | Δ⁹-THCP | 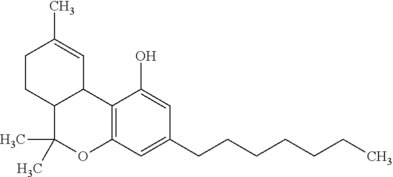 [SMILES: CCCCCCCc1cc(O)c2c(c1)OC(C)(C)C1CCC(C)=CC21] |
| cannabichromevarinic acid | CBCVA | 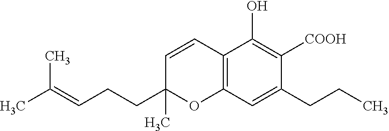 [SMILES: CCCc1cc2c(c(O)c1C(=O)O)C=CC(C)(CCC=C(C)C)O2] |
| cannabichromevarin | CBCV | 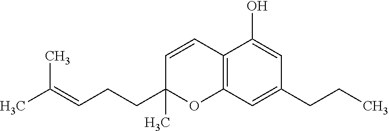 [SMILES: CCCc1cc(O)c2c(c1)OC(C)(CCC=C(C)C)C=C2] |
| cannabigerovarinic acid | CBGVA | 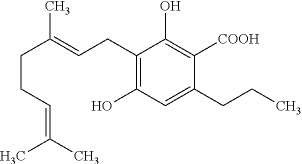 [SMILES: CCCc1cc(O)c(C/C=C(\C)CCC=C(C)C)c(O)c1C(=O)O] |
| cannabigerovarin | CBGV | 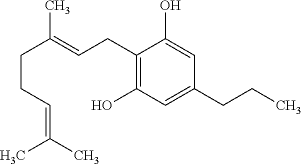 [SMILES: CCCc1cc(O)c(C/C=C(\C)CCC=C(C)C)c(O)c1] |
| cannabicyclolic acid | CBLA | 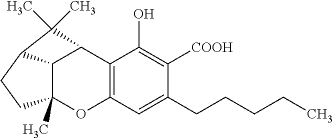 |
| cannabicyclol | CBL | 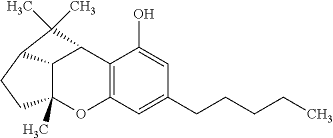 |
| cannabielsoinic acid | CBEA | 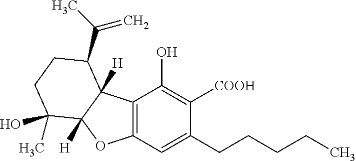 [SMILES: C=C(C)[C@@H]1CC[C@](C)(O)[C@H]2Oc3cc(CCCCC)c(C(=O)O)c(O)c3[C@@H]12] |
| cannabielsoin | CBE | 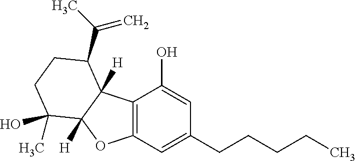 [SMILES: C=C(C)[C@@H]1CC[C@](C)(O)[C@H]2Oc3cc(CCCCC)cc(O)c3[C@@H]12] |
| cannabicitranic acid | CBTA | 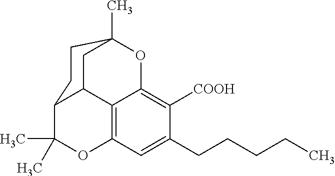 [SMILES: CCCCCc1cc2c3c(c1C(=O)O)OC1(C)CCC(C3C1)C(C)(C)O2] |
| cannabicitran | CBT | 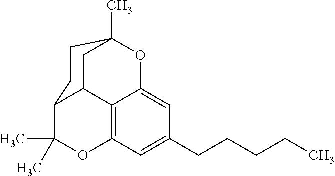 [SMILES: CCCCCc1cc2c3c(c1)OC(C)(C)C1CCC(C)(CC31)O2] |

“Cannabinoid precursor compound” or “cannabinoid precursor substrate” as used herein refers to a compound or molecule acted on by an enzyme in a biosynthetic step for producing a cannabinoid. Exemplary cannabinoid precursors are provided in Table 2, and include, but are not limited to, the aromatic polyketides, olivetolic acid (OA), and divarinic acid (DA), which are enzymatically prenylated with a geranyl group to form the cannabinoids, CBGA, and CBGVA, respectively.

TABLE 2
Exemplary cannabinoid precursor compounds

| | Abbrev. | |
|---|---|---|
| Compound Name | Name | Chemical Structure |
| 2,4-dihydroxy-6- methylbenzoic acid | | 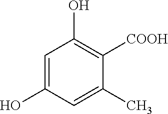 |
| 2-ethyl-4,6- dihydroxybenzoic acid | | 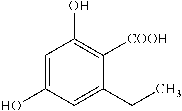 |
| Divarinic acid (2,4-dihydroxy-6- propylbenzoic acid) | DA | 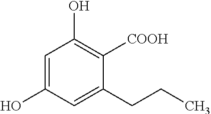 |
| Butolic acid (2-butyl-4,6- dihydroxybenzoic acid) | BA | 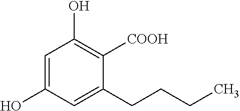 |
| Olivetolic acid (2,4-dihydroxy-6- pentylbenzoic acid) | OA | 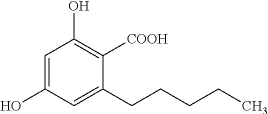 [SMILES: CCCCCc1cc(O)cc(O)c1C(=O)O] |
| 2-hexyl-4,6- dihydroxybenzoic acid | | 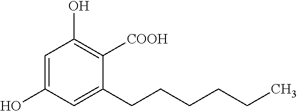 [SMILES: CCCCCCc1cc(O)cc(O)c1C(=O)O] |
| Phorolic acid (2-heptyl-4,6- dihydroxybenzoic acid) | PA | 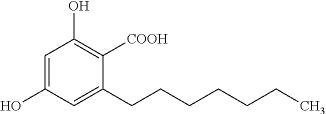 [SMILES: CCCCCCCc1cc(O)cc(O)c1C(=O)O] |

“Cyclolavandulyl-substituted cannabinoid” refers to a cannabinoid precursor compound modified with a cyclolavandulyl group. Typically, the cyclolavandulyl group modifies the cannabinoid precursor compound at a position where a prenyltransferase (e.g., NphB) enzymatically prenylates (e.g., with a geranyl group from GPP) the compound. Exemplary cyclolavandulyl-substituted cannabinoids of the present disclosure include but are not limited to the compounds having the chemical structures of compounds (2a), (2b), (2c), (2d), (2e), (2f), (2g), (2h), (2i), (2j), (2k), (2l), (2m), (2n), (2o), (2p), (2q), (2r), (2s), (2t), (2u), (2v), (2w), (2x), (2y), (2z), (2aa), (2bb), (2cc), (2dd), (2ee), (2ff), (2gg), (2hh), (2ii), (2jj), (2kk), (2ll), (2 mm), (2nn), (2oo), and (2pp), (including CBCLA, CBCL, CBCLVA, CBCLV, CBCLPA, and CPCLP) as depicted in Table 3.

TABLE 3
Exemplary cyclolavandulyl-substituted cannabinoids

| Compound | Chemical Structure |
|---|---|
| (2a) | 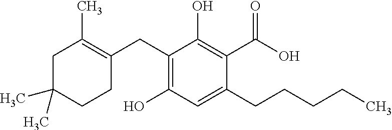 [SMILES: CCCCCc1cc(O)c(CC2=C(C)CC(C)(C)CC2)c(O)c1C(=O)O] CBCLA |
| (2b) | 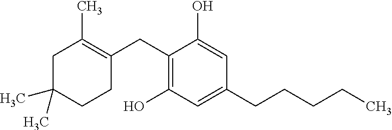 [SMILES: CCCCCc1cc(O)c(CC2=C(C)CC(C)(C)CC2)c(O)c1] CBCL |
| (2c) | 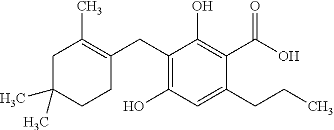 [SMILES: CCCc1cc(O)c(CC2=C(C)CC(C)(C)CC2)c(O)c1C(=O)O] CBCLVA |
| (2d) | 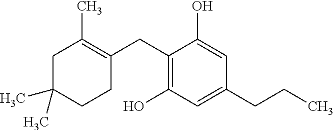 [SMILES: CCCc1cc(O)c(CC2=C(C)CC(C)(C)CC2)c(O)c1] CBCLV |
| (2e) | 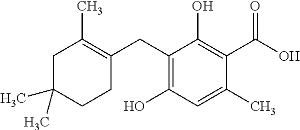 [SMILES: CC1=C(Cc2c(O)cc(C)c(C(=O)O)c2O)CCC(C)(C)C1] |
| (2f) | 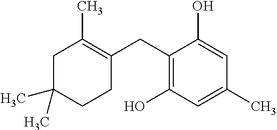 |
| (2g) | 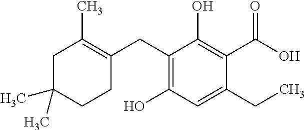 [SMILES: CCc1cc(O)c(CC2=C(C)CC(C)(C)CC2)c(O)c1C(=O)O] |
| (2h) | 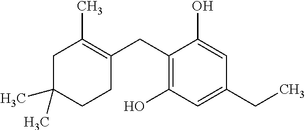 [SMILES: CCc1cc(O)c(CC2=C(C)CC(C)(C)CC2)c(O)c1] |
| (2i) | 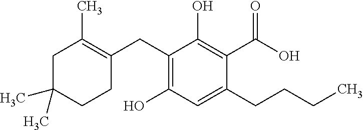 [SMILES: CCCCc1cc(O)c(CC2=C(C)CC(C)(C)CC2)c(O)c1C(=O)O] CBCLBA |
| (2j) | 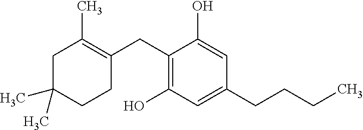 [SMILES: CCCCc1cc(O)c(CC2=C(C)CC(C)(C)CC2)c(O)c1] CBCLB |
| (2k) | 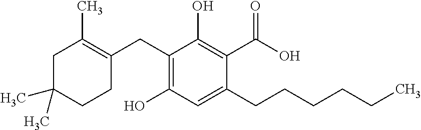 [SMILES: CCCCCCc1cc(O)c(CC2=C(C)CC(C)(C)CC2)c(O)c1C(=O)O] |
| (2l) | 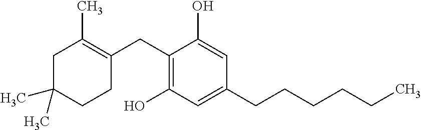 [SMILES: CCCCCCc1cc(O)c(CC2=C(C)CC(C)(C)CC2)c(O)c1] |
| (2m) | 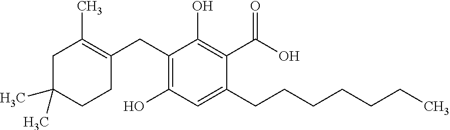 [SMILES: CCCCCCCc1cc(O)c(CC2=C(C)CC(C)(C)CC2)c(O)c1C(=O)O] CBCLPA |
| (2n) | 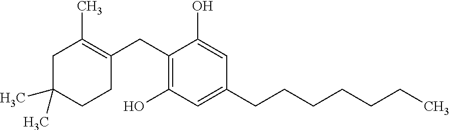 [SMILES: CCCCCCCc1cc(O)c(CC2=C(C)CC(C)(C)CC2)c(O)c1] CBCLP |
| (2o) | 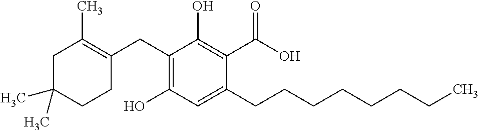 [SMILES: CCCCCCCCc1cc(O)c(CC2=C(C)CC(C)(C)CC2)c(O)c1C(=O)O] |
| (2p) | 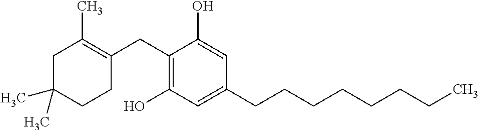 [SMILES: CCCCCCCCc1cc(O)c(CC2=C(C)CC(C)(C)CC2)c(O)c1] |
| (2q) | 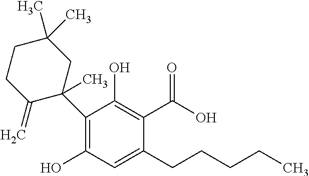 [SMILES: C=C1CCC(C)(C)CC1(C)c1c(O)cc(CCCCC)c(C(=O)O)c1O] |
| (2r) | 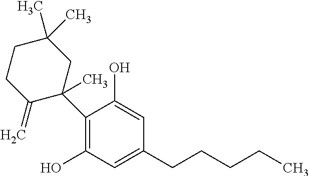 [SMILES: C=C1CCC(C)(C)CC1(C)c1c(O)cc(CCCCC)cc1O] |
| (2s) | 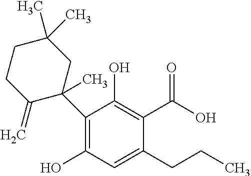 [SMILES: C=C1CCC(C)(C)CC1(C)c1c(O)cc(CCC)c(C(=O)O)c1O] |
| (2t) | 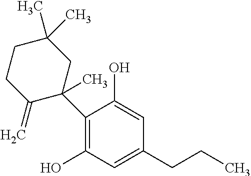 [SMILES: C=C1CCC(C)(C)CC1(C)c1c(O)cc(CCC)cc1O] |
| (2u) | 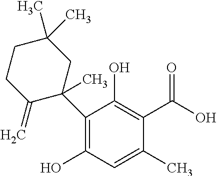 [SMILES: C=C1CCC(C)(C)CC1(C)c1c(O)cc(C)c(C(=O)O)c1O] |
| (2v) | 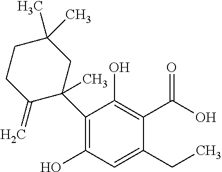 [SMILES: C=C1CCC(C)(C)CC1(C)c1c(O)cc(CC)c(C(=O)O)c1O] |
| (2w) | 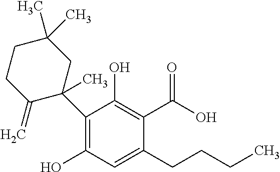 [SMILES: C=C1CCC(C)(C)CC1(C)c1c(O)cc(CCCC)c(C(=O)O)c1O] |
| (2x) | 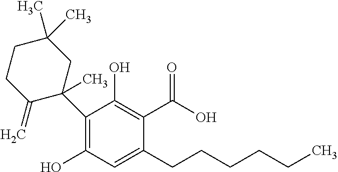 [SMILES: C=C1CCC(C)(C)CC1(C)c1c(O)cc(CCCCCC)c(C(=O)O)c1O] |
| (2y) | 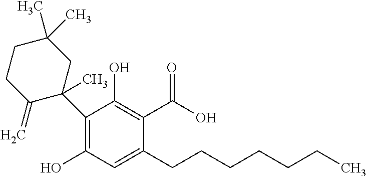 [SMILES: C=C1CCC(C)(C)CC1(C)c1c(O)cc(CCCCCCC)c(C(=O)O)c1O] |
| (2z) | 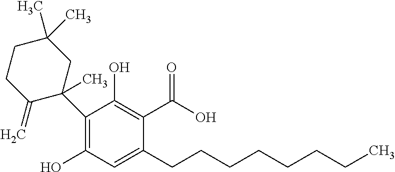 [SMILES: C=C1CCC(C)(C)CC1(C)c1c(O)cc(CCCCCCCC)c(C(=O)O)c1O] |
| (2aa) | 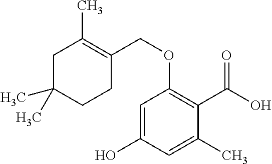 [SMILES: CC1=C(COc2cc(O)cc(C)c2C(=O)O)CCC(C)(C)C1] |
| (2bb) | 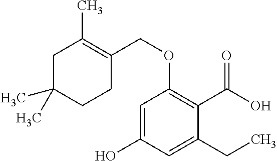 [SMILES: CCc1cc(O)cc(OCC2=C(C)CC(C)(C)CC2)c1C(=O)O] |
| (2cc) | 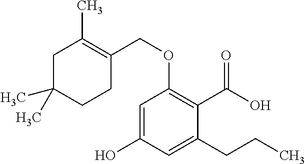 [SMILES: CCCc1cc(O)cc(OCC2=C(C)CC(C)(C)CC2)c1C(=O)O] |
| (2dd) | 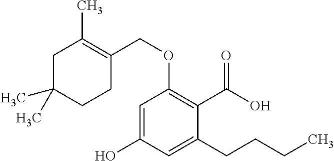 [SMILES: CCCCc1cc(O)cc(OCC2=C(C)CC(C)(C)CC2)c1C(=O)O] |
| (2ee) | 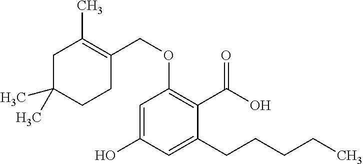 [SMILES: CCCCCc1cc(O)cc(OCC2=C(C)CC(C)(C)CC2)c1C(=O)O] |
| (2ff) | 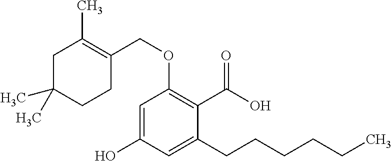 [SMILES: CCCCCCc1cc(O)cc(OCC2=C(C)CC(C)(C)CC2)c1C(=O)O] |
| (2gg) | 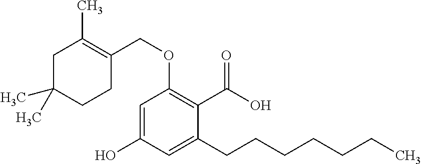 [SMILES: CCCCCCCc1cc(O)cc(OCC2=C(C)CC(C)(C)CC2)c1C(=O)O] |
| (2hh) | 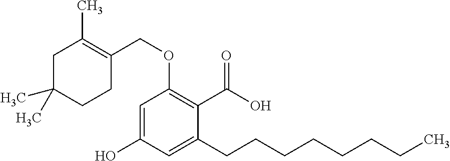 [SMILES: CCCCCCCCc1cc(O)cc(OCC2=C(C)CC(C)(C)CC2)c1C(=O)O] |
| (2ii) | 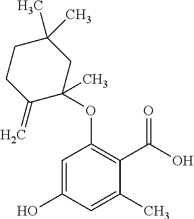 [SMILES: C=C1CCC(C)(C)CC1(C)Oc1cc(O)cc(C)c1C(=O)O] |
| (2jj) | 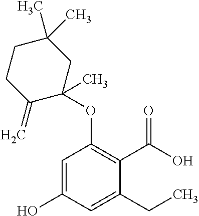 [SMILES: C=C1CCC(C)(C)CC1(C)Oc1cc(O)cc(CC)c1C(=O)O] |
| (2kk) | 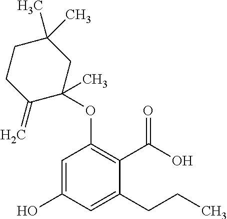 [SMILES: C=C1CCC(C)(C)CC1(C)Oc1cc(O)cc(CCC)c1C(=O)O] |
| (2ll) | 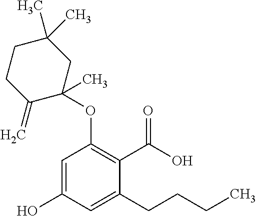 [SMILES: C=C1CCC(C)(C)CC1(C)Oc1cc(O)cc(CCCC)c1C(=O)O] |
| (2mm) | 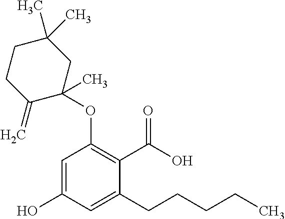 [SMILES: C=C1CCC(C)(C)CC1(C)Oc1cc(O)cc(CCCCC)c1C(=O)O] |
| (2nn) | 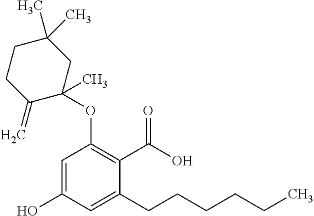 [SMILES: C=C1CCC(C)(C)CC1(C)Oc1cc(O)cc(CCCCCC)c1C(=O)O] |
| (2oo) | 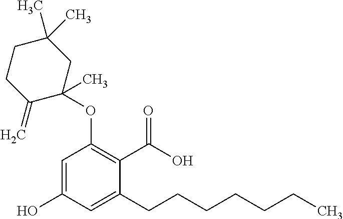 [SMILES: C=C1CCC(C)(C)CC1(C)Oc1cc(O)cc(CCCCCCC)c1C(=O)O] |
| (2pp) | 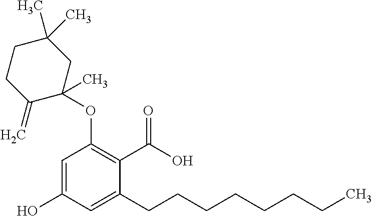 [SMILES: C=C1CCC(C)(C)CC1(C)Oc1cc(O)cc(CCCCCCCC)c1C(=O)O] |

“Flavonoid compound” refers to a compound of the class of polyphenolic secondary metabolite compounds found in plants, including, but not limited to, compounds in the following families: flavones, flavonols, flavanones, flavanonols, flavans, isoflavonoids. Exemplary flavonoids useful in the compositions and methods of the present disclosure include but are not limited to: Luteolin, Apigenin, Tangeritin, Quercetin, Kaempferol, Myricetin, Fisetin, Galangin, Isorhamnetin, Pachypodol, Rhamnazin, Pyranoflavonols, Furanoflavonols, Hesperetin, Naringenin, Eriodictyol, and Homoeriodictyol.

“Aromatic compound” refers to a compound that has at least one aromatic group capable of accepting a cyclolavandulyl group transfer from cyclolavandulyl pyrophosphate mediated by a prenyltransferase (e.g., NphB). Typically, such aromatic compounds are also capable of accepting transfer of a prenyl group (e.g., with a geranyl group from GPP) mediated by a prenyl transferase. Exemplary aromatic compounds of the present disclosure include but are not limited to the compounds of structural formulas III, IV, V, and VI, as disclosed elsewhere herein, and include, but are not limited to compounds of the chemical structures depicted in Table 4.

TABLE 4
Exemplary aromatic compounds
Chemical Structure 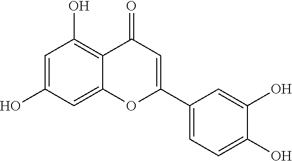 [SMILES: O=c1cc(-c2ccc(O)c(O)c2)oc2cc(O)cc(O)c12]
2-(3,4-dihydroxyphenyl)-5,7-dihydroxy-4H-1-benzopyran-4-one 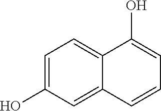 [SMILES: Oc1ccc2c(O)cccc2c1]
naphthalene-1,6-diol 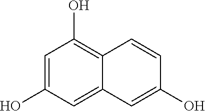 [SMILES: Oc1ccc2c(O)cc(O)cc2c1]
naphthalene-1,3,6-triol 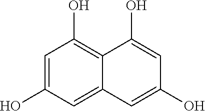 [SMILES: Oc1cc(O)c2c(O)cc(O)cc2c1]
naphthalene-1,3,6,8-tetrol 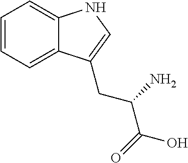 [SMILES: N[C@@H](Cc1c[nH]c2ccccc12)C(=O)O]
L-tryptophan 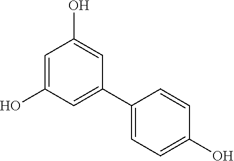 [SMILES: Oc1ccc(-c2cc(O)cc(O)c2)cc1]
[1,1′-biphenyl]-3,4′,5-triol 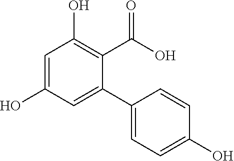 [SMILES: O=C(O)c1c(O)cc(O)cc1-c1ccc(O)cc1]
3,4′,5-trihydroxy[1,1′-biphenyl]-2-carboxylic acid 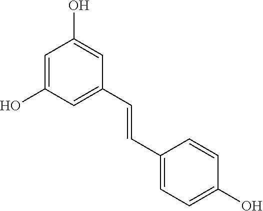 [SMILES: Oc1ccc(/C=C/c2cc(O)cc(O)c2)cc1]
5-[(E)-2-(4-hydroxyphenyl)ethenyl]benzene-1,3-diol 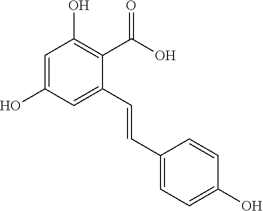 [SMILES: O=C(O)c1c(O)cc(O)cc1/C=C/c1ccc(O)cc1]
2,4-dihydroxy-6-[(E)-2-(4-hydroxyphenyl)ethenyl]benzoic acid

“Cyclolavandulyl-substituted aromatic compound” refers to an aromatic compound (e.g., cannabinoid precursor, alkaloid, or flavonoid compound) modified with a cyclolavandulyl group. Typically, the cyclolavandulyl group modifies the aromatic compound at a position where a prenyltransferase (e.g., NphB) enzymatically prenylates (e.g., with a geranyl group from GPP) the compound. Exemplary cyclolavandulyl-substituted aromatic compounds of the present disclosure include but are not limited to, the exemplary cyclolavandulyl-substituted cannabinoids of Table 3, and the cyclolavandulyl-substituted aromatic compounds (4a), (4b), (5a), (5b), (6a), (6b), (7a), (7b), (8a), (8b), (2qq), (2rr), (2ss), (2tt), (2uu), (2vv), (2ww), and (2xx) having the chemical structures depicted in Table 5.

TABLE 5
Exemplary cyclolavandulyl-substituted aromatic compounds 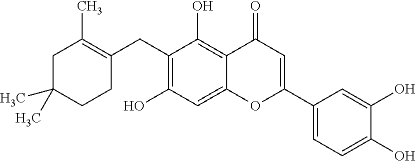 [SMILES: CC1=C(Cc2c(O)cc3oc(-c4ccc(O)c(O)c4)cc(=O)c3c2O)CCC(C)(C)C1]
(4a) 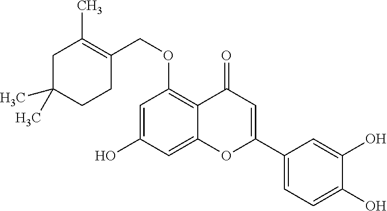 [SMILES: CC1=C(COc2cc(O)cc3oc(-c4ccc(O)c(O)c4)cc(=O)c23)CCC(C)(C)C1]
(4b) 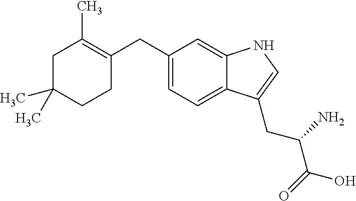 [SMILES: CC1=C(Cc2ccc3c(C[C@H](N)C(=O)O)c[nH]c3c2)CCC(C)(C)C1]
(5a) 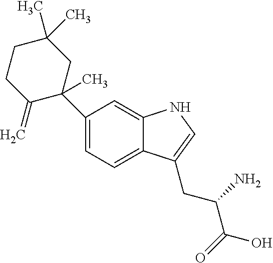 [SMILES: C=C1CCC(C)(C)CC1(C)c1ccc2c(C[C@H](N)C(=O)O)c[nH]c2c1]
(5b) 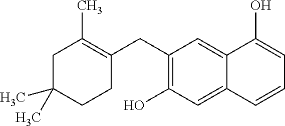 [SMILES: CC1=C(Cc2cc3c(O)cccc3cc2O)CCC(C)(C)C1]
(6a) 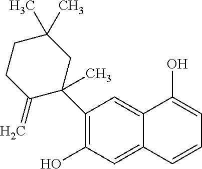 [SMILES: C=C1CCC(C)(C)CC1(C)c1cc2c(O)cccc2cc1O]
(6b) 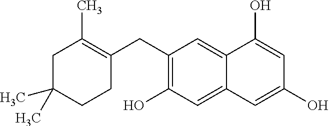 [SMILES: CC1=C(Cc2cc3c(O)cc(O)cc3cc2O)CCC(C)(C)C1]
(7a) 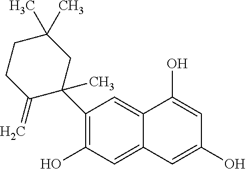 [SMILES: C=C1CCC(C)(C)CC1(C)c1cc2c(O)cc(O)cc2cc1O]
(7b) 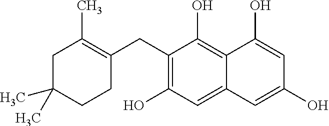 [SMILES: CC1=C(Cc2c(O)cc3cc(O)cc(O)c3c2O)CCC(C)(C)C1]
(8a) 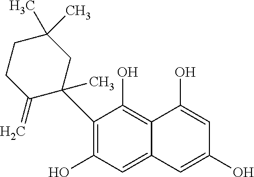 [SMILES: C=C1CCC(C)(C)CC1(C)c1c(O)cc2cc(O)cc(O)c2c1O]
(8b) 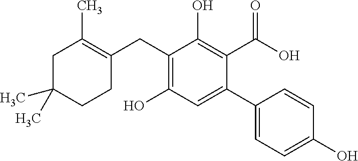 [SMILES: CC1=C(Cc2c(O)cc(-c3ccc(O)cc3)c(C(=O)O)c2O)CCC(C)(C)C1]
(2qq) 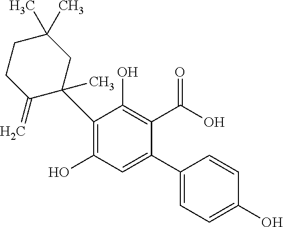 [SMILES: C=C1CCC(C)(C)CC1(C)c1c(O)cc(-c2ccc(O)cc2)c(C(=O)O)c1O]
(2rr) 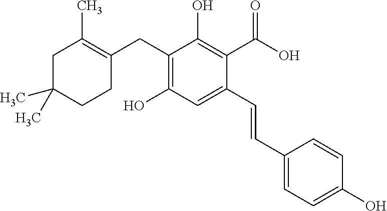 [SMILES: CC1=C(Cc2c(O)cc(/C=C/c3ccc(O)cc3)c(C(=O)O)c2O)CCC(C)(C)C1]
(2ss) 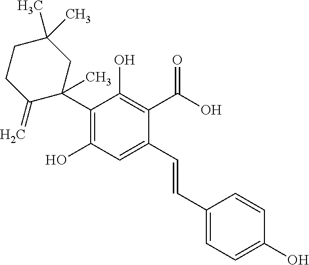 [SMILES: C=C1CCC(C)(C)CC1(C)c1c(O)cc(/C=C/c2ccc(O)cc2)c(C(=O)O)c1O]
(2tt) 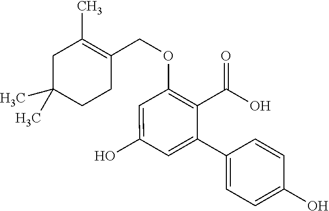 [SMILES: CC1=C(COc2cc(O)cc(-c3ccc(O)cc3)c2C(=O)O)CCC(C)(C)C1]
(2uu) 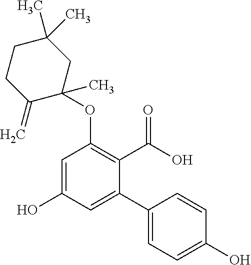 [SMILES: C=C1CCC(C)(C)CC1(C)Oc1cc(O)cc(-c2ccc(O)cc2)c1C(=O)O]
(2vv) 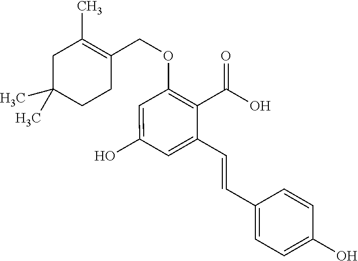 [SMILES: CC1=C(COc2cc(O)cc(/C=C/c3ccc(O)cc3)c2C(=O)O)CCC(C)(C)C1]
(2ww) 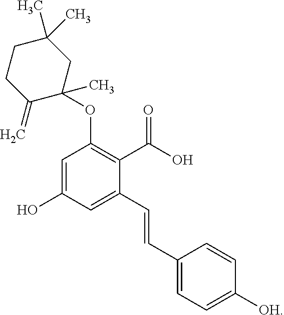 [SMILES: C=C1CCC(C)(C)CC1(C)Oc1cc(O)cc(/C=C/c2ccc(O)cc2)c1C(=O)O]
(2xx)

“Conversion” as used herein refers to the enzymatic conversion of the substrate(s) to the corresponding product(s). “Percent conversion” refers to the percent of the substrate that is converted to the product within a period of time under specified conditions. Thus, the “enzymatic activity” or “activity” of an enzymatic conversion can be expressed as “percent conversion” of the substrate to the product.

“Product” as used herein in the context of an enzyme mediated process refers to the compound or molecule resulting from the activity of the enzyme. In the context of the enzymes with CLDS activity useful in the methods and compositions of the present disclosure, an exemplary product is the cyclolavandulyl pyrophosphate (compound (1)) as shown in Scheme 1. In the context of the enzymes with PT activity useful in the methods and compositions of the present disclosure, exemplary products are the cyclolavandulyl-substituted aromatic compound listed in Tables 3 and 5.

“Substrate” as used herein in the context of an enzyme mediated process refers to the compound(s) or molecule(s) acted on by the enzyme. In the context of the enzymes with CLDS activity useful in the compositions and methods the present disclosure, substrates acted on by the enzyme can include dimethylallyl pyrophosphate (DMAPP) (see e.g., Scheme 1) and derivatives thereof. Similarly, in the context of the enzymes with PT activity, substrates acted on by the enzymes include the cyclolavandulyl group donor, cyclolavandulyl pyrophosphate (compound (1)) and the range of cyclolavandulyl acceptor aromatic compounds, including but not limited to, aromatic polyketides, such as cannabinoid precursor compounds of Table 2 (e.g., olivetolic acid), alkaloid compounds, flavonoid compounds, and other aromatic compounds of Table 4, including the exemplary aromatic compounds described elsewhere herein including the examples.

“Host cell” as used herein refers to a cell capable of being functionally modified with recombinant nucleic acids and functioning to express recombinant products, including polypeptides and compounds produced by activity of the polypeptides.

“Nucleic acid,” or “polynucleotide” as used herein interchangeably to refer to two or more nucleosides that are covalently linked together. The nucleic acid may be wholly comprised ribonucleosides (e.g., RNA), wholly comprised of 2′-deoxyribonucleotides (e.g., DNA) or mixtures of ribo- and 2′-deoxyribonucleosides. The nucleoside units of the nucleic acid can be linked together via phosphodiester linkages (e.g., as in naturally occurring nucleic acids), or the nucleic acid can include one or more non-natural linkages (e.g., phosphorothioester linkage). Nucleic acid or polynucleotide is intended to include single-stranded or double-stranded molecules, or molecules having both single-stranded regions and double-stranded regions. Nucleic acid or polynucleotide is intended to include molecules composed of the naturally occurring nucleobases (i.e., adenine, guanine, uracil, thymine, and cytosine), or molecules comprising that include one or more modified and/or synthetic nucleobases, such as, for example, inosine, xanthine, hypoxanthine, etc.

“Protein,” “polypeptide,” and “peptide” are used herein interchangeably to denote a polymer of at least two amino acids covalently linked by an amide bond, regardless of length or post-translational modification (e.g., glycosylation, phosphorylation, lipidation, myristilation, ubiquitination, etc.). As used herein “protein” or “polypeptide” or “peptide” polymer can include D- and L-amino acids, and mixtures of D- and L-amino acids.

“Naturally-occurring” or “wild-type” as used herein refers to the form as found in nature. For example, a naturally occurring nucleic acid sequence is the sequence present in an organism that can be isolated from a source in nature and which has not been intentionally modified by human manipulation.

“Recombinant,” “engineered,” or “non-naturally occurring” when used herein with reference to, e.g., a cell, nucleic acid, or polypeptide, refers to a material, or a material corresponding to the natural or native form of the material, that has been modified in a manner that would not otherwise exist in nature, or is identical thereto but is produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

“Nucleic acid derived from” as used herein refers to a nucleic acid having a sequence at least substantially identical to a sequence of found in naturally in an organism. For example, cDNA molecules prepared by reverse transcription of mRNA isolated from an organism, or nucleic acid molecules prepared synthetically to have a sequence at least substantially identical to, or which hybridizes to a sequence at least substantially identical to a nucleic sequence found in an organism.

“Coding sequence” refers to that portion of a nucleic acid (e.g., a gene) that encodes an amino acid sequence of a protein.

“Heterologous nucleic acid” as used herein refers to any polynucleotide that is introduced into a host cell by laboratory techniques and includes polynucleotides that are removed from a host cell, subjected to laboratory manipulation, and then reintroduced into a host cell.

“Codon optimized” refers to changes in the codons of the polynucleotide encoding a protein to those preferentially used in a particular organism such that the encoded protein is efficiently expressed in the organism of interest. Although the genetic code is degenerate in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some embodiments, the polynucleotides encoding the imine reductase enzymes may be codon optimized for optimal production from the host organism selected for expression.

“Preferred, optimal, high codon usage bias codons” refers to codons that are used at higher frequency in the protein coding regions than other codons that code for the same amino acid. The preferred codons may be determined in relation to codon usage in a single gene, a set of genes of common function or origin, highly expressed genes, the codon frequency in the aggregate protein coding regions of the whole organism, codon frequency in the aggregate protein coding regions of related organisms, or combinations thereof. Codons whose frequency increases with the level of gene expression are typically optimal codons for expression. A variety of methods are known for determining the codon frequency (e.g., codon usage, relative synonymous codon usage) and codon preference in specific organisms, including multivariate analysis, for example, using cluster analysis or correspondence analysis, and the effective number of codons used in a gene (see GCG CodonPreference, Genetics Computer Group Wisconsin Package; CodonW, John Peden, University of Nottingham; Mclnerney, J. O, 1998, Bioinformatics 14:372-73; Stenico et al., 1994, Nucleic Acids Res. 222437-46; Wright, F., 1990, Gene 87:23-29). Codon usage tables are available for a growing list of organisms (see for example, Wada et al., 1992, Nucleic Acids Res. 20:2111-2118; Nakamura et al., 2000, Nucl. Acids Res. 28:292; Duret, et al., supra; Henaut and Danchin, “Escherichia coli and Salmonella,” 1996, Neidhardt, et al. Eds., ASM Press, Washington D.C., p. 2047-2066. The data source for obtaining codon usage may rely on any available nucleotide sequence capable of coding for a protein. These data sets include nucleic acid sequences actually known to encode expressed proteins (e.g., complete protein coding sequences-CDS), expressed sequence tags (ESTS), or predicted coding regions of genomic sequences (see for example, Mount, D., Bioinformatics: Sequence and Genome Analysis, Chapter 8, Cold Spring Harbor Laboratory Press, Cold Spring Harbor, N.Y., 2001; Uberbacher, E. C., 1996, Methods Enzymol. 266:259-281; Tiwari et al., 1997, Comput. Appl. Biosci. 13:263-270).

“Control sequence” as used herein refers to all sequences, which are necessary or advantageous for the expression of a polynucleotide and/or polypeptide as used in the present disclosure. Each control sequence may be native or foreign to the nucleic acid sequence encoding a polypeptide. Such control sequences include, but are not limited to, a leader, a promoter, a polyadenylation sequence, a pro-peptide sequence, a signal peptide sequence, and a transcription terminator. At a minimum, control sequences typically include a promoter, and transcriptional and translational stop signals. The control sequences may be provided with linkers for the purpose of introducing specific restriction sites facilitating ligation of the control sequences with the coding region of the nucleic acid sequence encoding a polypeptide.

“Operably linked” as used herein refers to a configuration in which a control sequence is appropriately placed (e.g., in a functional relationship) at a position relative to a polynucleotide sequence or polypeptide sequence of interest such that the control sequence directs or regulates the expression of the sequence of interest.

“Promoter sequence” refers to a nucleic acid sequence that is recognized by a host cell for expression of a polynucleotide of interest, such as a coding sequence. The promoter sequence contains transcriptional control sequences, which mediate the expression of a polynucleotide of interest. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

“Percentage of sequence identity,” “percent sequence identity,” “percentage homology,” or “percent homology” are used interchangeably herein to refer to values quantifying comparisons of the sequences of polynucleotides or polypeptides, and are determined by comparing two optimally aligned sequences over a comparison window, wherein the portion of the polynucleotide or polypeptide sequence in the comparison window may comprise additions or deletions (or gaps) as compared to the reference sequence for optimal alignment of the two sequences. The percentage values may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman, 1981, Adv. Appl. Math. 2:482, by the homology alignment algorithm of Needleman and Wunsch, 1970, J. Mol. Biol. 48:443, by the search for similarity method of Pearson and Lipman, 1988, Proc. Natl. Acad. Sci. USA 85:2444, by computerized implementations of these algorithms (GAP, BESTFIT, FASTA, and TFASTA in the GCG Wisconsin Software Package), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)). Examples of algorithms that are suitable for determining percent sequence identity and sequence similarity are the BLAST and BLAST 2.0 algorithms, which are described in Altschul et al., 1990, J. Mol. Biol. 215:403-410 and Altschul et al., 1977, Nucleic Acids Res. 3389-3402, respectively. Software for performing BLAST analyses is publicly available through the National Center for Biotechnology Information website. This algorithm involves first identifying high scoring sequence pairs (HSPs) by identifying short words of length W in the query sequence, which either match or satisfy some positive-valued threshold score T when aligned with a word of the same length in a database sequence. T is referred to as, the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff and Henikoff, 1989, Proc Natl Acad Sci USA 89:10915). Exemplary determination of sequence alignment and % sequence identity can employ the BESTFIT or GAP programs in the GCG Wisconsin Software package (Accelrys, Madison Wis.), using default parameters provided.

“Reference sequence” refers to a defined sequence used as a basis for a sequence comparison. A reference sequence may be a subset of a larger sequence, for example, a segment of a full-length nucleic acid or polypeptide sequence. A reference sequence typically is at least 20 nucleotide or amino acid residue units in length but can also be the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides or polypeptides over a “comparison window” to identify and compare local regions of sequence similarity. “Comparison window” refers to a conceptual segment of at least about 20 contiguous nucleotide positions or amino acids residues wherein a sequence may be compared to a reference sequence of at least 20 contiguous nucleotides or amino acids and wherein the portion of the sequence in the comparison window may comprise additions or deletions (or gaps) of 20 percent or less as compared to the reference sequence (which does not comprise additions or deletions) for optimal alignment of the two sequences.

“Substantial identity” or “substantially identical” refers to a polynucleotide or polypeptide sequence that has at least 70% sequence identity, at least 80% sequence identity, at least 85% sequence identity, at least 90% sequence identity, at least 95% sequence identity, or at least 99% sequence identity, as compared to a reference sequence over a comparison window of at least 20 nucleoside or amino acid residue positions, frequently over a window of at least 30-50 positions, wherein the percentage of sequence identity is calculated by comparing the reference sequence to a sequence that includes deletions or additions which total 20 percent or less of the reference sequence over the window of comparison.

“Corresponding to,” “reference to,” or “relative to” when used in the context of the numbering of a given amino acid or polynucleotide sequence refers to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an engineered imine reductase, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned.

“Isolated” as used herein in reference to a molecule means that the molecule (e.g., cannabinoid, polynucleotide, polypeptide) is substantially separated from other compounds that naturally accompany it, e.g., protein, lipids, and polynucleotides. The term embraces nucleic acids which have been removed or purified from their naturally-occurring environment or expression system (e.g., host cell or in vitro synthesis).

“Substantially pure” refers to a composition in which a desired molecule is the predominant species present (i.e., on a molar or weight basis it is more abundant than any other individual macromolecular species in the composition) and is generally a substantially purified composition when the object species comprises at least about 50 percent of the macromolecular species present by mole or % weight.

“Recovered” as used herein in relation to an enzyme, protein, or cannabinoid compound, refers to a more or less pure form of the enzyme, protein, or cannabinoid.

### Biosynthetic Methods and Compositions for Preparing Cyclolavandulyl-Substituted Aromatic Compounds

Enzymes with prenyltransferase (PT) activity are capable of transferring a prenyl group from certain prenyl group donor substrates (e.g., geranyl pyrophosphate or “GPP”) to compounds capable of acting as prenyl group acceptor substrates, including a wide range of aromatic compounds such as flavonoids, alkaloids, and cannabinoid precursors. See e.g., Johnson et al., “Acceptor substrate determines donor specificity of an aromatic prenyltransferase: expanding the biocatalytic potential of NphB,” Applied Microbiology and Biotechnology (2020) 104:4383-4395. Scheme 1 (below) illustrates an exemplary prenyltransferase catalyzed transfer of the geranyl group of GPP to the aromatic polyketide cannabinoid precursor, olivetolic acid (OA), to form the cannabinoid, CBGA.

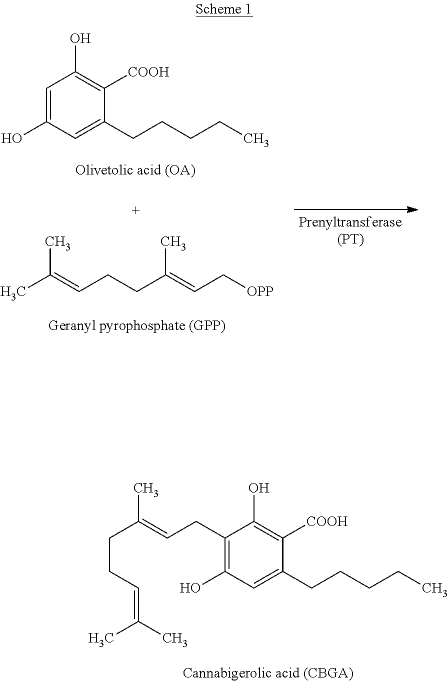

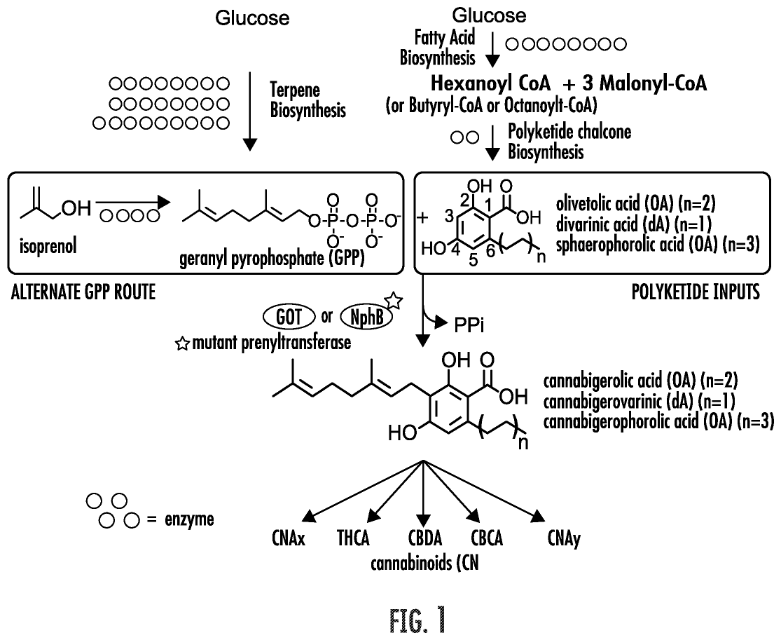

FIG. 1 depicts an illustrative schematic of the molecular inputs/outputs and enzymatic pathways in cannabinoid biosynthesis leading to the enzymatic prenylation step where a prenyltransferase (or “GOT”) (e.g., NphB) catalyzes the transfer of the geranyl group of GPP to an aromatic polyketide (e.g., olivetolic acid) that results in the formation of the prenylated aromatic cannabinoid product (e.g., CBGA).

Enzymes with cyclolavandulyl diphosphate synthase (CLDS) activity are capable of catalyzing the conversion of two molecules dimethylallyl pyrophosphate (DMAPP) to the product compound, cyclolavandulyl pyrophosphate (CLPP) (compound (1)) as shown in Scheme 2 below.

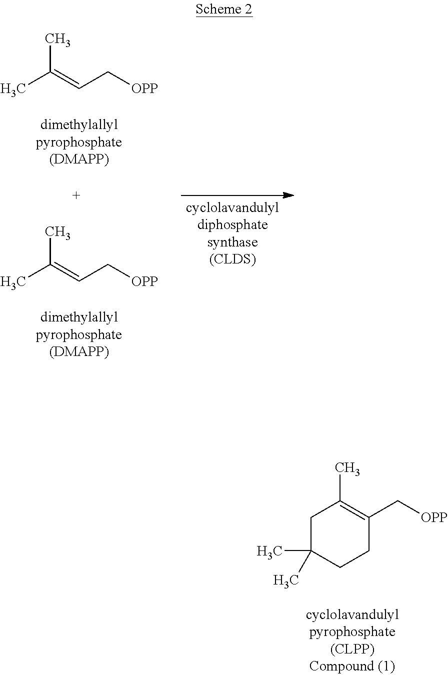

A naturally-occurring enzyme with CLDS activity from Streptomyces sp. CL190, has been isolated and structurally characterized. See, e.g., Ozaki et al., “Cyclolavandulyl Skeleton Biosynthesis via Both Condensation and Cyclization Catalyzed by an Unprecedented Member of the cis-Isoprenyl Diphosphate Synthase Superfamily,” J. Am. Chem. Soc. 2014, 136, 4837-4840; Tomita et al., “Structure and mechanism of the monoterpene cyclolavandulyl diphosphate synthase that catalyses consecutive condensation and cyclisation,” Angew. Chemie. Int'l Ed. 2017, 10.1002/anie201708474. The polypeptide sequence of the CLDS from Streptomyces sp. CL190 (GenBank accession: BAO66170.1; PDB: 5YGJ_A) has 217 amino acids and is provided herein as SEQ ID NO: 2:

(SEQ ID NO: 2)
MTTLMLLPDGMRRWSEKNGVSLDDGYAAMGDKIIEFMGWAKEEGVKTLYI
TASSAANHGRPEAAVNTFMEAFTEVIRRCHSQFKFDFSGSLDLVSEDYLT
ELSALRDKSDSESDFTLHYILGMSLSHEVVGIFNKLNGKIPEMTEEILAE
NAYVPTQVDYIIRTGGAIRMSSFFPLMSPYAELHFSPVLFPDTTRADFDA
ALKDLRARDRRFGGYPA

It is a surprising discovery of the present disclosure that a cyclolavandulyl group donor compound, such as CLPP (compound (1)), can be used as a donor substrate by enzymes with PT activity (e.g., NphB) in the biosynthesis of various cyclolavandulyl-substituted aromatic compounds, such as cyclolavandulylated cannabinoid, CBCLA of compound (2e) as shown in Scheme 3 below:

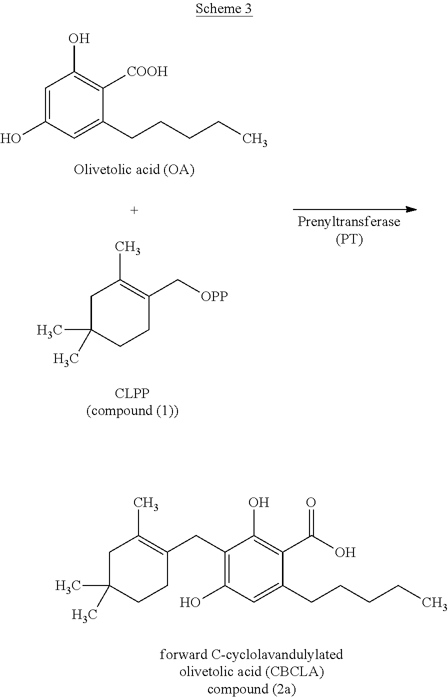

The discovery that enzymes with PT activity can catalyze the transfer of a cyclolavandulyl to an aromatic compound use of enzymes provides for novel biosynthetic methods and compositions useful for the preparation of cyclolavandulyl-substituted aromatic compounds. In at least one embodiment, the present disclosure contemplates a method for preparing a cyclolavandulyl-substituted aromatic compound like that illustrated in Scheme 3. Such a method comprises a biocatalytic step of contacting the cyclolavandulyl donor compound, CLPP (compound (1)) with an aromatic compound (e.g., OA), and prenyltransferase, under suitable conditions, and then recovering the enzymatic product of a cyclolavandulyl-substituted aromatic compound (e.g., compound (2e)) from the reaction mixture. In such a method, the reaction can be carried out using in vitro enzymatic reaction conditions like those typically used for a prenyltransferase reaction with using GPP as the prenyl donor rather than the cyclolavandulyl donor compound, CLPP (compound (1)).

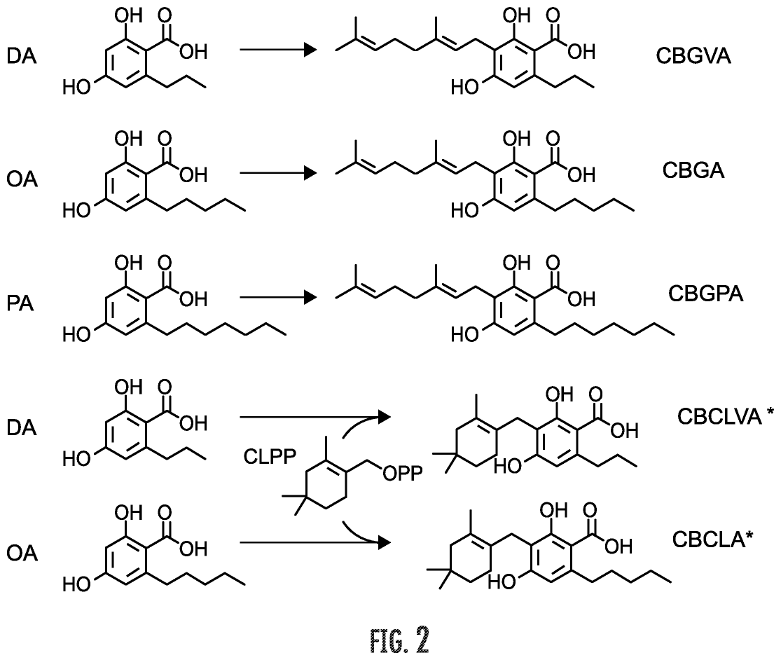

As a comparison, FIG. 2 depicts the biosynthetic prenylation reactions producing the standard cannabinoids, CBGVA, CBGA, and CBGPA, and the alternative biosynthetic cyclolavandulylation reactions that can be carried out using CLPP as the cyclolavandulyl group donor to the cannabinoid precursor substrate that results in production of the cyclolavandulyl-cannabinoid compounds, CBCLVA, and CBCLA.

Without intending to be bound by mechanism, it is well established that prenylation as catalyzed by prenyltransferases occurs in two steps: (1) formation of the resonance stabilized allylic cation; and (2) nucleophilic attack by the activated aromatic substrate. See e.g., Tanner, “Mechanistic studies on the indole prenyltransferases,” Natural Product Reports, Issue 1, 2015; doi.org/10.1039/C4NP00099D. Following dissociation of the prenyl group donor pyrophosphate (e.g., GPP) in the active site of a prenyltransferase, the resulting primary allylic carbocation is stabilized through resonance with the tertiary carbocation. This allylic cation is prone to nucleophilic attack at both the primary (C1) position as well as the tertiary (C3) position as dictated by the active site architecture of the prenyltransferase. “Forward” prenylation is said to occur if the nucleophile attacks the C1 position, whereas “reverse” prenylation is said to occur if the nucleophile attacks the C3 position. See e.g., Walsh “Biological Matching of Chemical Reactivity: Pairing Indole Nucleophilicity with Electrophilic Isoprenoids,” ACS Chem. Biol. 2014, 9, 12, 2718-2728; doi.org/10.1021/cb500695k. Generally, the nucleophile must either be an indole or an activated benzene. The stereoselectivity and regioselectivity (which atom —C, —O, or —N in the activated substrate) is dictated by active site architecture and can be readily modulated via protein engineering. See e.g., Yang et al., “Catalytic Mechanism of Aromatic Prenylation by NphB,” Biochemistry 2012, 51, 12, 2606-2618; Fan et al., “Site-directed Mutagenesis Switching a Dimethylallyl Tryptophan Synthase to a Specific Tyrosine C3-Prenylating Enzyme,” J. Biol. Chem. 290 (3), 16 Jan. 2015, 1364-1373; Valliere et al., “A cell-free platform for the prenylation of natural products and application to cannabinoid production,” Nature Communications 10, 565 (2019); DOI: 10.1038/s41467-019-08448-y. Owing to the electron rich nature of aromatic hydroxyls, both forward and reverse prenylation at the hydroxyl positions is widely observed in natural products (Ref 6, Ref 7, Ref 8).

Accordingly, it is contemplated that cyclolavandulylation of an aromatic catalyzed by an aromatic prenyltransferase as disclosed herein can provide both “forward” products (as shown in Scheme 3) or “reverse” products as shown in the exemplary Scheme 4 below.

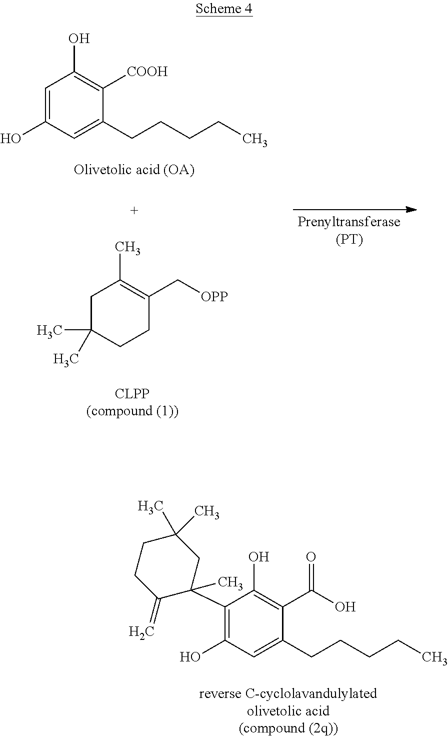

It also is contemplated that cyclolavandulylation of an aromatic compound catalyzed by an aromatic prenyltransferase as disclosed herein can provide both “forward” and “reverse” products at the hydroxyl position resulting cyclolavandulated aromatic produces as shown in the exemplary Scheme 5 and Scheme 6 below.

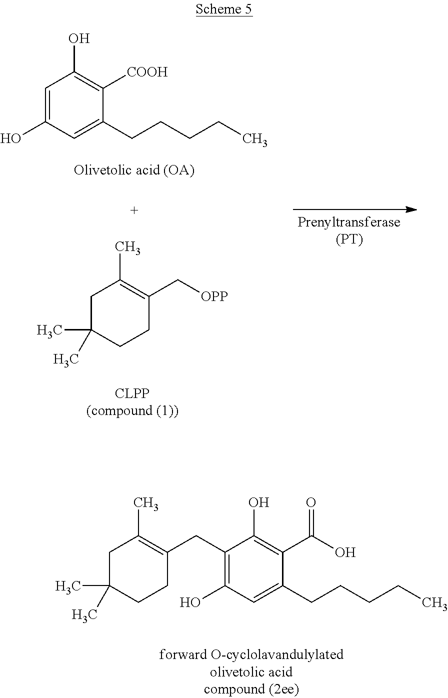

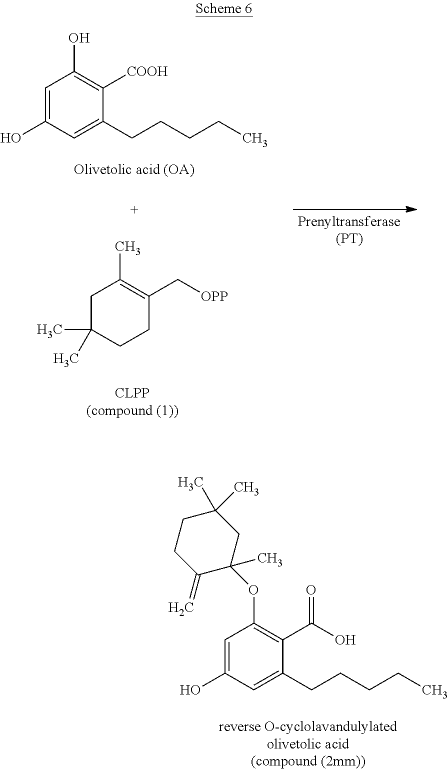

Accordingly, in at least one embodiment, the present disclosure provides a method for the biosynthesis of a cyclolavandulyl-substituted aromatic compound comprising: (a) contacting in a reaction mixture under suitable reaction conditions, the cyclolavandulyl donor compound, CLPP (compound (1)), an enzyme with aromatic prenyltransferase activity (e.g., NphB or an NphB variant), and an aromatic compound capable of acting as a cyclolavandulyl acceptor substrate; and (b) recovering the cyclolavandulyl-substituted aromatic compound from the reaction mixture.

As described elsewhere herein, it is also contemplated that the enzymatic reactions of Schemes 3, 4, 5, and 6 that produce the exemplary carboxylated forms of cyclolavandulyl-substituted cannabinoid compounds (2a), (2q), (2ee), and (2 mm) can be extended to include a further step of decarboxylation and thereby produce the corresponding decarboxylated cannabinoid compound. For example, the reaction of Scheme 3 produces the carboxylated cyclolavandulyl-substituted cannabinoid compound (2a), which can be further decarboxylated to produce compound (2b).

In an alternative method contemplated by the present disclosure, rather than adding CLPP (compound (1)) as a reagent in the enzymatic reaction with a PT, this cyclolavandulyl donor compound can be prepared in situ using via the enzymatic reaction of DMAPP with an enzyme having CLDS activity (e.g., the reaction of Scheme 2). In other words, the CLPP generating enzymatic reaction of Scheme 2 can be combined with the PT-catalyzed cyclolavandulyl group transfer reaction, like that illustrated in Scheme 3. In such a method, two enzymes, one with CLDS activity, and one with PT activity, are combined in a reaction mixture with DMAPP and the aromatic compound that is the cyclolavandulyl group acceptor. Under the suitable reaction conditions, the cyclolavandulyl donor substrate, CLPP (compound (1)) is catalyzed in situ by the CLDS and then acts as the substrate with the enzyme having PT activity for transfer to the aromatic compound.

Accordingly, in at least one embodiment, the present disclosure provides a method for the biosynthesis of a cyclolavandulyl-substituted aromatic compound comprising: (a) contacting in a reaction mixture under suitable reaction conditions, an enzyme with cyclolavandulyl diphosphate synthase (CLDS) activity, a dimethylallyl pyrophosphate (DMAPP), an enzyme with aromatic prenyltransferase activity, and an aromatic compound capable of acting as a cyclolavandulyl acceptor substrate; and (b) recovering the cyclolavandulyl-substituted aromatic compound from the reaction mixture.

The compositions of enzymes and reactants used in the enzymatic reaction methods are also provided herein. Thus, in at least one embodiment the disclosure provides a composition comprising: the cyclolavandulyl donor compound, CLPP (compound (1)), an enzyme with aromatic prenyltransferase activity (e.g., NphB or an NphB variant), and an aromatic compound capable of acting as a cyclolavandulyl acceptor substrate. In another embodiment, the disclosure provides a composition comprising an enzyme with cyclolavandulyl diphosphate synthase (CLDS) activity, a dimethylallyl pyrophosphate (DMAPP), an enzyme with aromatic prenyltransferase activity, and an aromatic compound capable of acting as a cyclolavandulyl acceptor substrate.

As noted elsewhere herein, enzymes with PT activity have been known to transfer prenyl groups to a range of aromatic compounds, including aromatic polyketides, flavonoids, alkaloids, and aromatic amino acid analogs. The present disclosure contemplates that the promiscuity of enzymes with PT activity (e.g., NphB and its variants) with aromatic prenyl group acceptor compounds allows for cyclolavandulyl transfer to such a range of compounds in a similar manner. Accordingly, aromatic compounds capable of acting as cyclolavandulyl group acceptor substrates for biosynthesis of cyclolavandulyl-substituted aromatic products in the methods and compositions of the present disclosure include compounds of structural formula (III)

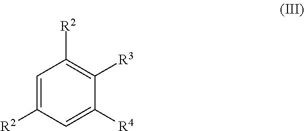

wherein, R² is —H or —OH, R³ is —H or —COOH; and R⁴ is linear or branched C1-C10 alkyl, linear or branched C1-C10 alkyl-amide, linear or branched C1-C10 alkyl-amine, linear or branched C1-C10 alkyl-alkylene, linear or branched C1-C10 alkyl-alkoxy, or C1-C10 alkyl-aryl, wherein any of the C1-C10 groups is optionally substituted with a —OH, —OCH₃, or a halogen. Exemplary aromatic polyketides represented by structural formula (III) that are capable of acting as cyclolavandulyl group acceptor substrates, include, but are not limited to, the cannabinoid precursor compounds of Table 2.

The cannabinoid precursor compounds (3a), (3b), (3c), (3d), (3e), (3f), (3g), and (3h) (see Table 2) can form a range of cannabinoid product compounds upon cyclolavandulylation with a suitable cyclolavandulyl donor, such as compound (1), via PT activity (e.g., NphB) to form a range cyclolavandulyl-substituted cannabinoid products of compounds (2a), (2b), (2c), (2d), (2e), (2f), (2g), (2h), (2i), (2j), (2k), (2l), (2m), (2n), (2o), (2p), (2q), (2r), (2s), (2t), (2u), (2v), (2w), (2x), (2y), (2z), (2aa), (2bb), (2cc), (2dd), (2ee), (2ff), (2gg), (2hh), (2ii), (2jj), (2kk), (2ll), (2 mm), (2nn), (2oo), and (2pp) (see e.g., compounds of Table 3).

It is further contemplated that the preparation of a carboxylated cyclolavandulyl-substituted cannabinoid compound of Table 3, can include a further step of decarboxylation to provide the corresponding decarboxylated cyclolavandulyl-substituted cannabinoid compound. For example, the carboxylated cyclolavandulyl-substituted cannabinoids of compound (2a) and (2c) can be decarboxylated to produce the cyclolavandulyl-substituted cannabinoids of compounds (2b) and (2d), respectively.

Other aromatic compounds capable of acting as cyclolavandulyl group acceptor substrates for enzymes with PT activity include compounds of structural formula (IV), (V), and (VI)

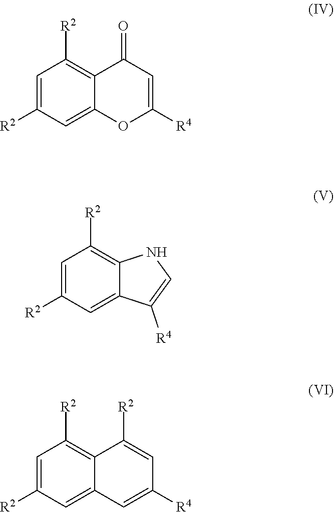

wherein, R² is —H or —OH, R³ is —H or —COOH; and R⁴ is —H, —OH, linear or branched C1-C10 alkyl, linear or branched C1-C10 alkyl-amide, linear or branched C1-C10 alkyl-amine, linear or branched C1-C10 alkyl-alkylene, linear or branched C1-C10 alkyl-alkoxy, or C1-C10 alkyl-aryl, wherein any of the C1-C10 groups is optionally substituted with a —OH, —OCH₃, or a halogen.

The compounds represented by structural formulas (IV), (V), and (VI) capable of acting as cyclolavandulyl group acceptor substrates, include but are not limited to, flavonoid compounds, alkaloid compounds, and other compounds listed in Table 4. The aromatic compounds of Table 4 can form a range of product compounds upon cyclolavandulylation with a suitable cyclolavandulyl donor, such as compound (1), via PT activity (e.g., NphB) to form a range cyclolavandulyl-substituted aromatic products including compounds (3i), (3j), (3k), (31), (4a), (5a), (6a), and (6b) (see e.g., compounds of Table 5).

In another embodiment, the present disclosure provides a method for preparing a cyclolavandulyl-substituted aromatic compound comprising: (a) contacting under suitable reaction conditions: cyclolavandulyl diphosphate synthase (CLDS), dimethylallyl pyrophosphate, a prenyltransferase, and an aromatic compound selected from a compound of structural formulas (III), (IV), (V), and (VI), wherein, R² is —H or —OH, R³ is —H or —COOH; and R⁴ is —H, —OH, linear or branched C1-C10 alkyl, linear or branched C1-C10 alkyl-amide, linear or branched C1-C10 alkyl-amine, linear or branched C1-C10 alkyl-alkylene, linear or branched C1-C10 alkyl-alkoxy, or C1-C10 alkyl-aryl, wherein any of the C1-C10 groups is optionally substituted with a —OH, —OCH₃, or a halogen; and (b) recovering the cyclolavandulyl-substituted aromatic compound from the reaction mixture.

As noted elsewhere herein, the naturally occurring cyclolavandulyl diphosphate synthase (CLDS) from Streptomyces CL190 is capable of converting two molecules of DMAPP to CLPP (compound (1)). In at least one embodiment of the method, the cyclolavandulyl diphosphate synthase (CLDS) is a polypeptide comprising an amino acid sequence of the CLDS of SEQ ID NO: 2. It is also contemplated that variants of the naturally occurring CLDS of SEQ ID NO: 2 can be prepared that have improved properties for use in the methods and compositions of the present disclosure. For example, a CLDS variant with increased thermostability and/or solubility for use in cell-free reaction methods. Accordingly, in at least one embodiment of the methods or composition, an enzyme with CLDS activity can be used wherein the enzyme is a variant of the naturally occurring CLDS of SEQ ID NO: 2, for example, wherein the variant comprises an amino acid sequence having at least 90%, at least 95%, at least 97%, at least 98%, or at least 99% identity to SEQ ID NO: 2. In at least one embodiment, the CLDS is modified with a HIS tag, such as the CLDS of SEQ ID NO: 4.

The naturally occurring prenyltransferase, NphB from Streptomyces sp. CL190, which has the amino acid sequence of SEQ ID NO: 8, has been engineered to provide soluble variants, such as NphBM31s (SEQ ID NO: 6), with PT activity capable of prenylating the aromatic polyketides, OA, or DA with GPP to form the cannabinoid compounds, CBGA or CBGVA, respectively. See e.g., WO2019173770A1; WO2019183152A1; WO2020028722A1, and WO2021134024A1, each of which is hereby incorporated by reference herein. These engineered NphB variants can be used in cell-free biosynthesis systems and methods for the preparation of cannabinoid compounds. See e.g., WO2020028722A1 and WO2021134024A1. The variant, NphBM31s (SEQ ID NO: 6) an exemplary enzyme with PT activity useful in the methods and compositions of the present disclosure. Specific protocols and conditions for the use of NphB, NphBM31s, other NphB variants, and other PTs are provided in the Examples and elsewhere herein. Accordingly, in at least one embodiment of any of the methods or compositions for preparing a cyclolavandulyl-substituted aromatic compounds disclosed herein, the enzyme with prenyltransferase activity used is NphB, or a variant of NphB; optionally, wherein the NphB or variant of NphB comprises an amino acid sequence having at least 90%, at least 95%, at least 97%, at least 98%, or at least 99% identity to NphB (SEQ ID NO: 8) or NphBM31s (SEQ ID NO: 6), or another variant of NphB or NphBM31s, such as any of variants disclosed in WO2021134024A1, which is hereby incorporated by reference herein.

Aromatic prenyltransferases other than NphB from Streptomyces sp. CL190 are known and contemplated for use in the methods and compositions for preparing cyclolavandulyl substituted aromatic compounds of the present disclosure. Screening, such as described in the examples of the present disclosure, can be used to identify useful prenyltransferases. Accordingly, in at least one embodiment, the methods and compositions of the present disclosure can be carried out using a naturally occurring prenyltransferase comprising an amino acid selected from SEQ ID NO: 9, 10, 11, 12, 13, 14, 15, 16, 17, and 18. In another embodiment, it is contemplated that variants of these prenyltransferases can be engineered and screened for activity in transferring a cyclolavandulyl group to an aromatic compound acceptor. Accordingly, in at least one embodiment, the methods and compositions of the present disclosure can be carried out using a variant of a prenyltransferase comprising an amino acid selected from SEQ ID NO: 9, 10, 11, 12, 13, 14, 15, 16, 17, and 18, wherein the variant comprises an amino acid sequence having at least 90%, at least 95%, at least 97%, at least 98%, or at least 99% identity to SEQ ID NO: 9, 10, 11, 12, 13, 14, 15, 16, 17, and 18. In at least one embodiment, the prenyltransferase comprises an amino acid sequence having at least 90%, at least 95%, at least 97%, at least 98%, or at least 99% identity to SEQ ID NO: 6, 8, 9, 14, 17, 18, or 19.

As noted elsewhere herein, the transfer of a prenyl group from a donor substrate, such as geranyl pyrophosphate (GPP) to an aromatic polyketide compound is a critical enzymatic step in the biosynthesis of many compounds of interest, including cannabinoids. Accordingly, it is contemplated that the methods and compositions of the present disclosure that use a prenyltransferase activity to catalyze the transfer of a cyclolavandulyl group to an aromatic acceptor compound can be used in a range of in vitro, cell-free systems, or in vivo, recombinant host cell systems for the biosynthesis of cyclolavandulylated aromatic compounds that are normally synthesized via a prenyltransferase step. FIG. 1 depicts a schematic overview of the molecular inputs/outputs and enzymes involved in such an exemplary system that utilizes a prenyltransferase in the biosynthesis of cannabinoid compounds. In the right side of the scheme of FIG. 1, the input molecule glucose is converted via fatty acid biosynthesis enzymes to the precursor compounds, hexanoyl-CoA, and malonyl-CoA. Or alternatively to hexanoyl-CoA, butyryl-CoA, or octanoyl-CoA. The precursors, hexanoyl-CoA and malonyl-CoA are converted via polyketide chalcone biosynthesis enzymes to the cannabinoid precursor compounds, olivetolic acid (OA). Or alternatively, butyryl-CoA is converted to divarinic acid (DA), or the precursor octanoyl-CoA is converted to sphaerophorolic acid (PA). Each of the cannabinoid precursors in this scheme, OA, DA, or PA, is capable as acting as a cannabinoid precursor substrate compound, or as the aromatic cyclolavandulyl group acceptor in the methods of the present disclosure. The left side of this scheme of FIG. 1, depicts a terpene biosynthesis route that converts glucose input molecules to geranyl pyrophosphate (GPP), which is the co-substrate used by the engineered NphB to convert the cannabinoid precursor, OA, DA, or PA, to the corresponding cannabinoid product compounds, CBGA, CBGVA, or CBGPA. It is contemplated that this terpene biosynthesis route can be abbreviated with the production of the cyclolavandulyl precursor, DMAPP, and thereby incorporated into the methods of the present disclosure. As shown in the scheme of FIG. 1, the cannabinoid products are themselves precursor substrate compounds that can be converted by cannabinoid synthase enzyme to the cannabinoids, THCA, CBDA, CBCA, and other structural analogs.

In at least one embodiment, the methods, and compositions for the biosynthesis of cyclolavandulyl-substituted aromatic compounds the present disclosure can be used in cell-free, in vitro biosynthesis of cyclolavandulyl-substituted cannabinoid compounds. Cell free cannabinoid biosynthesis methods utilizing the soluble prenyltransferase, NphB, are described in Valliere et al. “A bio-inspired cell-free system for cannabinoid production from inexpensive inputs,” Nature Chemical Biology Vol. 16, December 2020, 1427-1433; WO2020028722A1, and WO2021134024A1, each of which is hereby incorporated by reference herein. The increased thermostability of some of the engineered NphB variant polypeptides disclosed in e.g., WO2021134024A1, allows them to be incorporated directly into the cell-free cyclolavandulyl-substituted cannabinoid biosynthesis methods of the present disclosure. Moreover, use of these engineered NphB variant polypeptides can allow for higher temperatures resulting higher rates of conversion.

In at least one embodiment, the present disclosure provides a cell-free biosynthetic reaction scheme and system for the production of a range of cyclolavandulyl-substituted aromatic compounds, including cannabinoids, flavonoids, alkaloids, and other cyclolavandulylated products using the methods and compositions of the present disclosure. In at least one embodiment, the cell-free biosynthetic reaction scheme provides a pathway for production of DMAPP, and the production of an enzyme with CLDS activity that converts the DMAPP to CLPP. In at least one embodiment, the cell-free biosynthetic reaction scheme also provides a pathway for the production of the aromatic cyclolavandulyl-acceptor substrate, e.g., a cannabinoid precursor such as OA, DA, or PA. In at least one embodiment, the cell-free biosynthetic reaction schemes also incorporate the soluble prenyltransferase NphB, or one of its variants, as the enzyme with PT activity for transfer of the cyclolavandulyl group to the aromatic compound acceptor in the reaction mixture. The use of a cell-free biosynthetic scheme and system can simplify optimization of the biosynthesis, by allowing facile modification or addition pathway enzymes and modification of reagents or co-factors.

As illustrated by the exemplary cannabinoid biosynthetic reaction scheme of FIG. 1, the input compounds hexanoyl-CoA and malonyl-CoA can be used as substrates in a cell-free biosynthesis pathway for production of the cannabinoid precursor compound, olivetolic acid (OA). This biosynthesis begins with the condensation of hexanoyl-CoA and malonyl-CoA catalyzed by olivetol synthase (OLS) (BAG14339.1 from C. sativa) to generate 3,5,7-trioxododecanoyl-CoA. The enzyme, olivetolic acid cyclase (OAC) (AFN42527.1 from C. sativa) cyclizes 3,5,7-trioxododecanoyl-CoA to OA. Similar biosynthesis pathways can lead to the OA analogs, DA, and PA. The prenyl donor substrate, GPP, is produced via terpene biosynthesis enzyme pathways. In some instances, the enzymatic pathway steps may utilize co-factors (e.g., NAD(P)H, ATP/ADP etc.). Table 6 provides a list of exemplary enzymes that can be used in a cell-free biosynthesis system incorporating the recombinant prenyltransferase polypeptides of the present disclosure.

TABLE 6
Enzymes useful in a cell-free enzymatic system

| Enzyme | | | |
|---|---|---|---|
| Abbreviation | Enzyme | Source Organism | Accession # |
| AAE3 | Acyl Activating Enzyme 3 | C. sativa | AFD33347.1 |
| MatB | Malonyl-CoA Synthetase | R. plaustris | CAE25665.1 |
| mdcA | Malonate Decarboxylase α | Geobacillus sp. 44B | OQO99201.1 |
| | subunit | | |
| PTA | Phosphotransacetylase | G. stearothermophilus | WP_053532564 |
| OLS | Olivetol Synthase | C. sativa | BAG14339.1 |
| OAC | Olivetolic Acid Cyclase | C. sativa | AFN42527.1 |
| ADK | Adenylate Kinase | G. thermodenitrificans | ABO65513 |
| Ppase | Pyrophosphatase | G. stearothermophilus | O05724 |
| CPK | Creatine Kinase | Rabbit Muscle | Sigma Aldrich |
| ThiM | Hydroxyethylthiazole kinase | E. coli | NP_416607 |
| IPK | Isopentenyl Kinase | M. jannaschii | WP_01069535 |
| IDI | Isopentyl diphosphate | E. coli | NP_417365 |
| | isomerase | | |
| FPPS S82F | Farnesyl Pyrophosphate | G. stearothermophilus | KOR95521 |
| | Synthase | | |

The cell-free biosynthetic reactions using the recombinant polypeptides of the present disclosure can be carried out using a range of biocatalytic reaction methods. For example, the pathway enzymes can be purchased commercially, mixed in a suitable buffer with the recombinant prenyltransferase polypeptides of the present disclosure, and then the solution is exposed to the suitable substrate, and incubated under conditions suitable for production of the desired cannabinoid compound. In some embodiments, it is contemplated that one or more of the pathway enzymes can be bound to a solid support. It is also contemplated that one or more of the pathway enzymes can be expressed using phage display or other surface expression system and, for example, fixed in a fluid pathway corresponding to points in the metabolic pathway's cycle.

It is also contemplated that one or more polynucleotides encoding the one or more pathway enzymes can be cloned into one or more host cells under conditions providing expression of the pathway enzymes. The host cells can then be lysed and the lysate comprising the one or more enzymes (including the recombinant prenyltransferase polypeptides) can be combined with a suitable buffer and substrate (and one or more additional enzymes of the pathway, if necessary) to produce the desired cannabinoid. Alternatively, the enzymes can be isolated from the lysed preparations with or without heat treatment and then recombined in an appropriate buffer.

In one embodiment, the pathway enzymes, other than the PT and CLDS polypeptides of the present disclosure can derived from thermophilic microorganisms. The microorganisms are cultured to express the thermostable enzymes, then lysed, and the culture lysate heated to a temperature wherein the thermostable enzymes of the pathway remain active while other enzymes become inactive. Such a heat purified lysate preparation can then be used together with the PT and/or CLDS polypeptides of the present disclosure in a cell-free biosynthesis reaction to produce a desired cyclolavandulyl-substituted aromatic compound.

In at least one embodiment of a method for producing a cyclolavandulyl-substituted aromatic compound (e.g., cannabinoid), a heterologous nucleic acid encoding a recombinant polypeptide having CLDS activity and increased thermostability, (e.g., a thermostable CLDS variant) can be introduced into a recombinant host cell. The recombinant host cell can then be used for production of the polypeptide, or incorporated in a biocatalytic process that utilized the CLDS activity of the recombinant polypeptide expressed by the host cell for the catalytic preparation of the cyclolavandulyl group donor substrate, CLPP, used by a PT to produce cyclolavandulyl-substituted cannabinoid, CBCLA. In at least one embodiment, the recombinant host cell can further comprise a pathway of enzymes capable of producing a cannabinoid (e.g., CBGA) in addition to the recombinant polypeptide with CLDS activity. It is contemplated that a recombinant host cell comprising a heterologous nucleic acid encoding a recombinant polypeptide of the present disclosure can provide improved biosynthesis of a CBCLA in terms of titer, yield, and production rate, due to the improved thermostability of the expressed activity.

Accordingly, in at least one embodiment, the present disclosure provides a method of producing a cyclolavandulyl-substituted aromatic compound, wherein the method comprises: (a) culturing in a suitable medium a recombinant host cell of the present disclosure; and (b) recovering the produced cyclolavandulyl-substituted derivative. In at least one embodiment, the method of producing a cyclolavandulyl-substituted derivative further contacting a cell-free extract of the culture containing the produced cyclolavandulyl-substituted aromatic compound with a biocatalytic reagent or chemical reagent capable of converting the compound to a further derivative compound. In at least one embodiment, the biocatalytic reagent is an enzyme capable of converting the produced cyclolavandulyl-substituted cannabinoid (e.g., CBCLA) to a different cyclolavandulyl-substituted cannabinoid compound (e.g., CBCL). In at least one embodiment, the chemical reagent is capable of chemically modifying the produced cyclolavandulyl-substituted cannabinoid to produce a different cyclolavandulyl-substituted cannabinoid. In at least one embodiment of the method for producing a cyclolavandulyl-substituted cannabinoid, the method can further comprise contacting a cell-free extract of the culture containing the produced cyclolavandulyl-substituted cannabinoid with a biocatalytic reagent or chemical reagent.

It is contemplated that the cyclolavandulyl-substituted cannabinoid, flavonoid, alkaloid, or other aromatic derivative produced using the methods and compositions of the present disclosure can be produced and/or recovered from the reaction in the form of a salt. In at least one embodiment, the recovered salt of the cyclolavandulyl-substituted cannabinoid, flavonoid, alkaloid, or other aromatic compound is a pharmaceutically acceptable salt. Such pharmaceutically acceptable salts retain the biological effectiveness and properties of the free base compound.

### EXAMPLES

Various features and embodiments of the disclosure are illustrated in the following representative examples, which are intended to be illustrative, and not limiting. Those skilled in the art will readily appreciate that the specific examples are only illustrative of the invention as described more fully in the claims which follow thereafter. Every embodiment and feature described in the application should be understood to be interchangeable and combinable with every embodiment contained within.

### Example 1: In Vitro Biosynthesis of a Cyclolavandulyl-Substituted Cannabinoid CBCLA

This example illustrates the preparation of the cyclolavandulyl-substituted cannabinoid compound, cannabicyclolavolic acid (CBCLA), via a cell-free biosynthesis reaction using a recombinantly produced cyclolavandulyl diphosphate synthase, CLDS, from Streptomyces sp. CL190 and the recombinant prenyltransferase, NphB.

### Materials and Methods

### A. Cloning and Expression of CLDS

The gene of GenBank accession AB872045.1 which encodes the CLDS from Streptomyces sp. CL190 (SEQ ID NO: 2) was codon-optimized and synthesized by Twist DNA with the addition of an N-terminus 6×-HIS tag for expression in Escherichia coli and received as a clonal gene in the pET28a expression vector. The expressed CLDS protein with HIS tag corresponds to SEQ ID NO: 4. The clonal gene in the pET28a expression vector was transformed into BL21-Gold (DE3) competent cells using standard chemical transformation methods. A single colony was used to inoculate 4 mL LB+kanamycin (50 μg/mL), which was grown at 37° C. and 250 rpm. After 12 hours, the overnight was used to inoculate 1 L LB+kanamycin (50 μg/mL). At an OD600 of ˜0.6, the culture was induced with the addition of 0.4 mM isopropyl β-d-1-thiogalactopyranoside (IPTG) and grown at 18° C. and 250 rpm. After 12 hours, protein purification was carried out using standard Ni-NTA methods.

B. Cell-Free Cannabinoid Derivative Biosynthesis Reaction with CLDS

Production of CBCLA using a cell-free biosynthesis reaction was carried out using a modified procedure similar to that described in Ozaki et al. (“Cyclolavandulyl Skeleton Biosynthesis via Both Condensation and Cyclization Catalyzed by an Unprecedented Member of the cis-Isoprenyl Diphosphate Synthase Superfamily,” J. Am. Chem. Soc. 2014, 136, 4837-4840) and Tomita et al., (“Structure and mechanism of the monoterpene cyclolavandulyl diphosphate synthase that catalyses consecutive condensation and cyclisation,” Angew. Chemie. Int'l Ed. 2017, 10.1002/anie201708474) with the prenyltransferase substrate, cyclolavandulyl-pyrophosphate (CLPP), first synthesized from DMAPP. Following CLPP synthesis, olivetolic acid (OA) and the prenyltransferase, NphBM31s were added to the solution to form the cyclolavandulyl derivative of OA. NphBM31s (SEQ ID NO: 6) is prepared by cloning and expression in the pET28 vector as described above for CLDS, or as described in WO2021134024A1. Briefly: 10 mM DMAPP, 5 mM MgCl₂, 100 mM Tris-HCl (pH=8), and 4 mg/mL CLDS (SEQ ID NO: 4) were combined in a 2.25 mL reaction, which was incubated at 28° C. for 48 hours. Then, 5 mM of the olivetolic acid and 1.26 mg/ml of NphBM31s were added to the reaction mixture, which was incubated at 28° C. for an additional 48 hours.

### C. HPLC Analysis of Biosynthesis Reaction Products

Analysis of the reaction mixture was carried out as follows: 50 UL of the reaction mixture were added to 1 mL MeOH. The resulting solution was vortexed, centrifuged at 17,200 g for 5 minutes, and transferred to an HPLC vial for analysis. The sample was analyzed using an UltiMate 3000 HPLC equipped with a 100×4.6 mm 3 μm Syncronis C8 column and a mobile phase consisting of H₂O (0.1% TFA) and ACN (0.1% TFA).

### Results

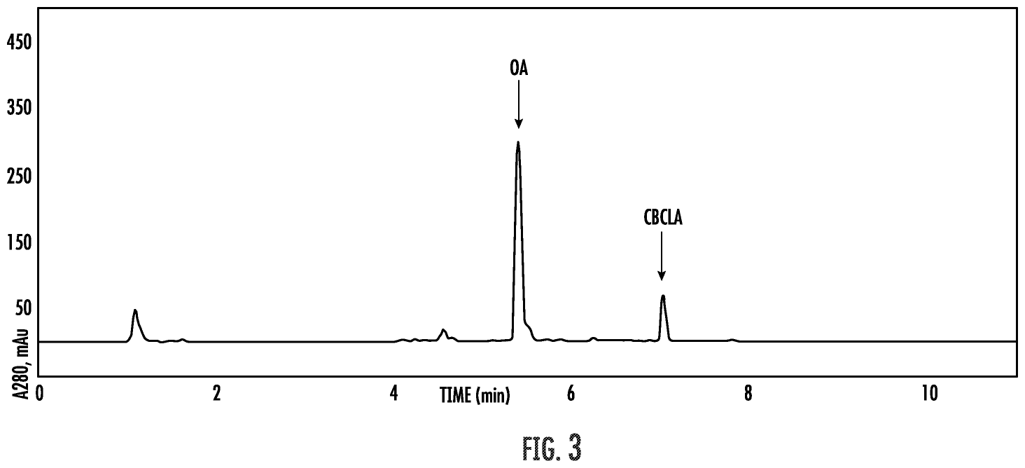

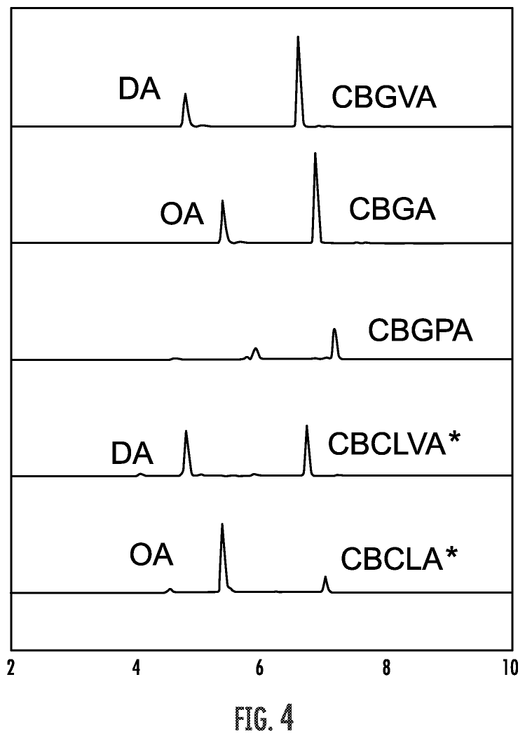

As shown by the HPLC chromatogram depicted in FIG. 3, the cyclolavandulyl cannabinoid compound, CBCLA was produced in the cell-free reaction at titer of ˜ 0.5 g/L. HPLC analysis confirmed that the compound is distinct from CBGA and that only a single, specific product is made. FIG. 4 shows exemplary LC-MS plots for the in vitro biosynthesis of the prenylated cannabinoids, CBGVA, CBGA, and CBGPA, for comparison with the LC-MS plots for the cyclolavandulylated cannabinoid compounds, CBCLVA, and CBCLA.

### Example 2: Biosynthesis of Cyclolavandulyl-Substituted Aromatic Compounds Using Various Prenyltransferases

This example illustrates in vitro cyclolavandulylation of various aromatic compounds in a cell-free biosynthesis reaction using a recombinantly produced cyclolavandulyl diphosphate synthase, CLDS (SEQ ID NO: 4) and a range of enzymes with prenyltransferase activity.

### Materials and Methods

### A. Preparation of Prenyltransferases

Genes encoding the following prenyltransferases were codon-optimized, synthesized, and cloned into the pET28a vector for protein expression and isolation/purification as described in Example 1.
NphB (Accession: 1ZB6_A) (SEQ ID NO: 8)
AbPT (Accession: KPI30840.1) (SEQ ID NO: 9)
SkPT (Accession: Q2L6E3.1) (SEQ ID NO: 10)
SvPT (Accession: WP_078899560.1) (SEQ ID NO: 11)
Sr1310PT (Accession: WP_057602682.1) (SEQ ID NO: 12)
NapT8 (Accession: ABS50461.1) (SEQ ID NO: 13)
NapT9 (Accession: ABS50490.1) (SEQ ID NO: 14)
Mcl23PT (Accession: AGH68908.1) (SEQ ID NO: 15)
CnqP3PT (Accession: WP_047018069.1) (SEQ ID NO: 16)
AtaWTPT (Accession: AMB20850.1) (SEQ ID NO: 17)
MzPT1 (Accession: WP_135330628.1) (SEQ ID NO: 18)
MzPT2 (Accession: WP_135330626.1) (SEQ ID NO: 19)
   B. Cell-Free Biosynthesis Reaction with CLPP and Various Aromatic Substrates

The cyclolavandulyl diphosphate synthase, CLDS was prepared as described in Example 1 and used in the synthesis of CLPP.

Stocks of the various aromatic compounds used as substrates for cyclolavandulylation activity screening were obtained from the following commercial sources:
Resveratrol (Fisher Scientific) 200 mM stock in DMSO
Luteolin (Fisher Scientific) 200 mM stock in DMSO
Naringenin (Fisher Scientific) 200 mM stock in DMSO
Tryptamine (Fisher Scientific) 200 mM stock in DMSO
Tryptophan (Fisher Scientific) 40 mM stock in water
Olivetolic acid (Toronto Research Chemicals) 200 mM stock in DMSO
Divarinic acid (Toronto Research Chemicals) 200 mM stock in DMSO
Phorolic acid (prepared in house) 200 mM stock in DMSO
Olivetol (Fisher Scientific) 200 mM stock in DMSO
Screening for cell-free biosynthesis was carried out as follows. 10 mM DMAPP, 5 mM MgCl₂, 100 mM Tris-HCl (pH=8), and 4 mg/mL CLDS (SEQ ID NO: 4) were combined in a 6.80 mL reaction, which was incubated at 28° C. for 48 hours. This mixture was aliquoted into a 96-well plate (80 μL per well). The various prenyltransferases (18 μL, ˜1 g/L final concentration) listed above were screened across various substrates (2 μL of stock solution, variable concentration shown above), which were added to the wells and incubated at 28° C. for an additional 48 hours.

### C. Analysis of Reaction Products

Analysis of the reaction mixture was carried out as follows: 50 μL of the reaction mixture were added to 1 mL MeOH. The resulting solution was vortexed, centrifuged at 17,200 g for 5 minutes, and transferred to an HPLC vial for analysis. HRMS-QTOF analyses of the cell free reactions was performed on an Agilent Technologies 6545 Accurate Mass QTOF LC/MS with a reverse-phase column (Agilent Infinity Lab Poroshell 120 ECC18, 2.7 μm, 3.0×50 mm) using positive ESI with 1% MeCN—H₂O (0.1% FA) for 2 min followed by a linear gradient of 1-99% MeCN—H₂O (0.1% FA) for 14 min with a flow rate of 0.4 mL/min.

Mass spectrometric (MS) analysis of chromatograms was carried out using MassHunter Qualitative Analysis 10.0. Targeted metabolomics using the exact m/z of the calculated proton adduct [H⁺] in the presence and absence of prenyltransferase indicated the presence of cyclolavandulylated product. Results are shown in Table 7.

TABLE 7

| | Aromatic Compound (Substrate) | | | | | | | | |
|---|---|---|---|---|---|---|---|---|---|
| Prenyl- | Res- | | Narin- | Trypt- | Trypt- | Olivetolic | Divarinic | Phorolic | |
| transferase | veratrol | Luteolin | genin | amine | ophan | Acid | Acid | Acid | Olivetol |
| NphBM31s | Yes | No | No | No | No | Yes | Yes | No | No |
| (SEQ ID NO: 6) | | | | | | | | | |
| NphB | No | No | Yes | No | Yes | No | — | — | — |
| (SEQ ID NO: 8) | | | | | | | | | |
| AbPT | No | No | No | No | Yes | No | — | — | — |
| (SEQ ID NO: 9) | | | | | | | | | |
| SkPT | No | No | No | No | No | No | — | — | — |
| (SEQ ID NO: 10) | | | | | | | | | |
| SvPT | No | No | No | No | No | No | — | — | — |
| (SEQ ID NO: 11) | | | | | | | | | |
| Sr1310PT | No | No | No | No | No | No | — | — | — |
| (SEQ ID NO: 12) | | | | | | | | | |
| NapT8 | No | No | No | No | No | No | — | — | — |
| (SEQ ID NO: 13) | | | | | | | | | |
| NapT9 | No | Yes | No | No | No | No | — | — | — |
| (SEQ ID NO: 14) | | | | | | | | | |
| Mcl23PT | No | No | No | No | No | No | — | — | — |
| (SEQ ID NO: 15) | | | | | | | | | |
| CnqP3PT | No | No | No | No | No | No | — | — | — |
| (SEQ ID NO: 16) | | | | | | | | | |
| AtaWTPT | Yes | No | Yes | No | Yes | No | — | — | — |
| (SEQ ID NO: 17) | | | | | | | | | |
| MzPT1 | No | No | No | No | No | Yes | — | — | — |
| (SEQ ID NO: 18 ) | | | | | | | | | |
| MzPT2 | No | No | No | No | No | No | — | — | — |
| (SEQ ID NO: 19) | | | | | | | | | |

### Results:

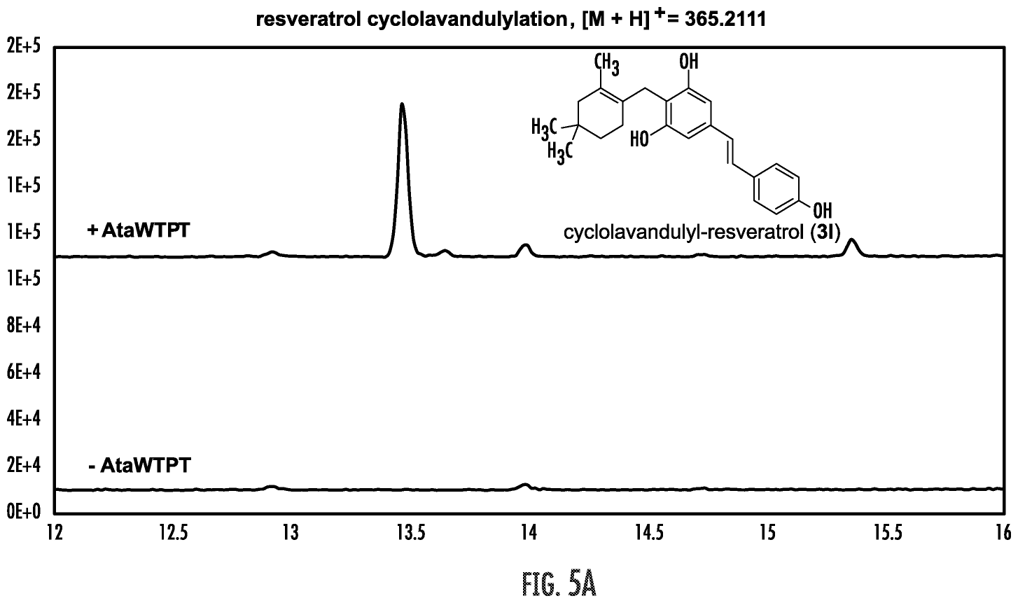

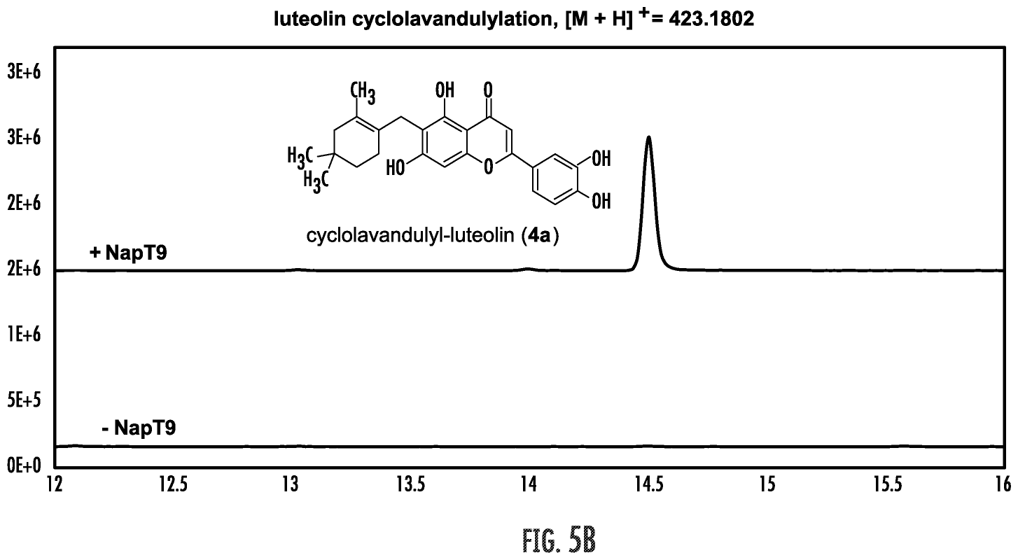

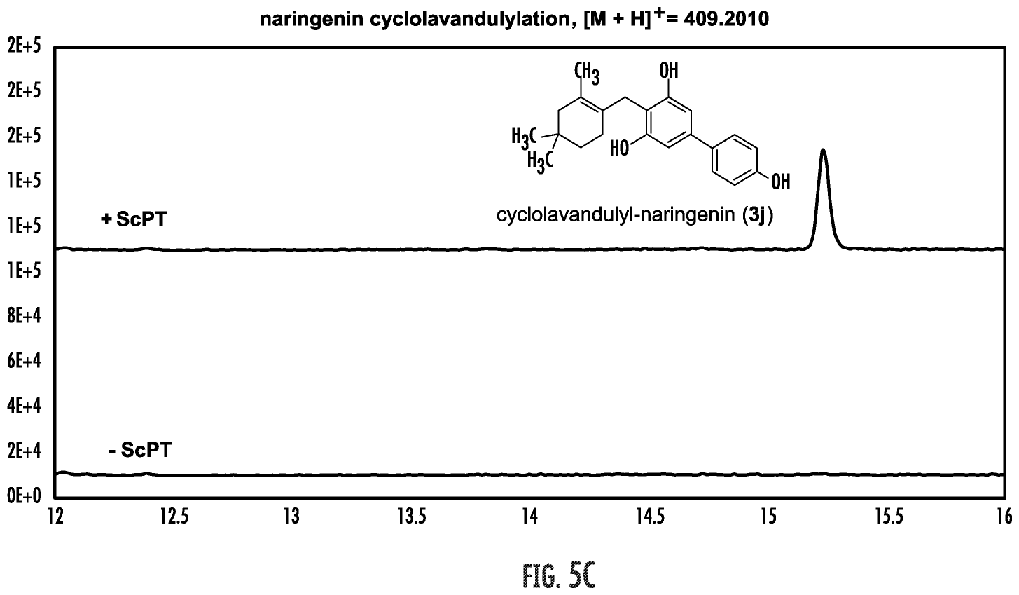

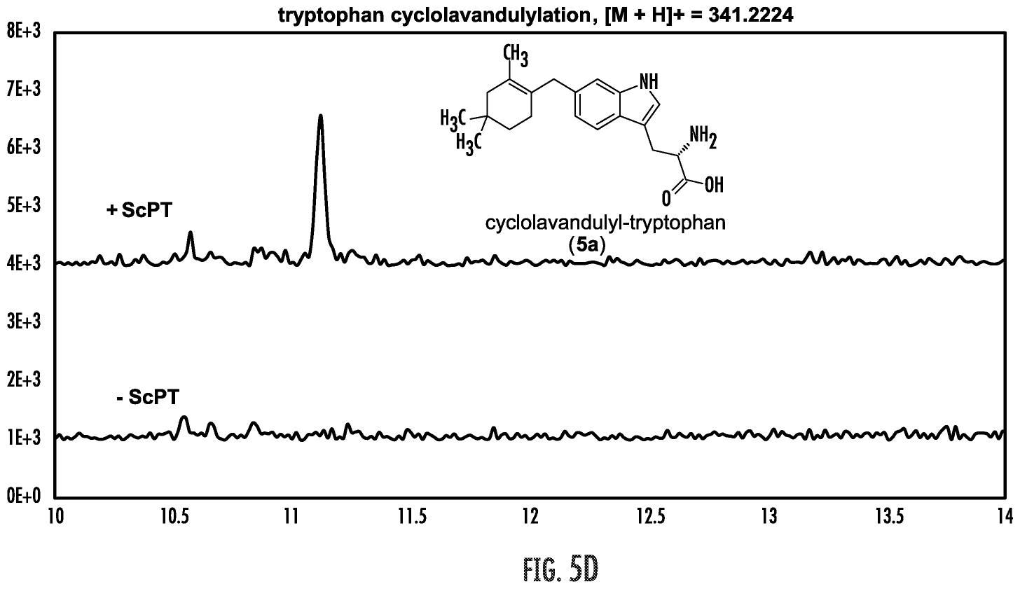

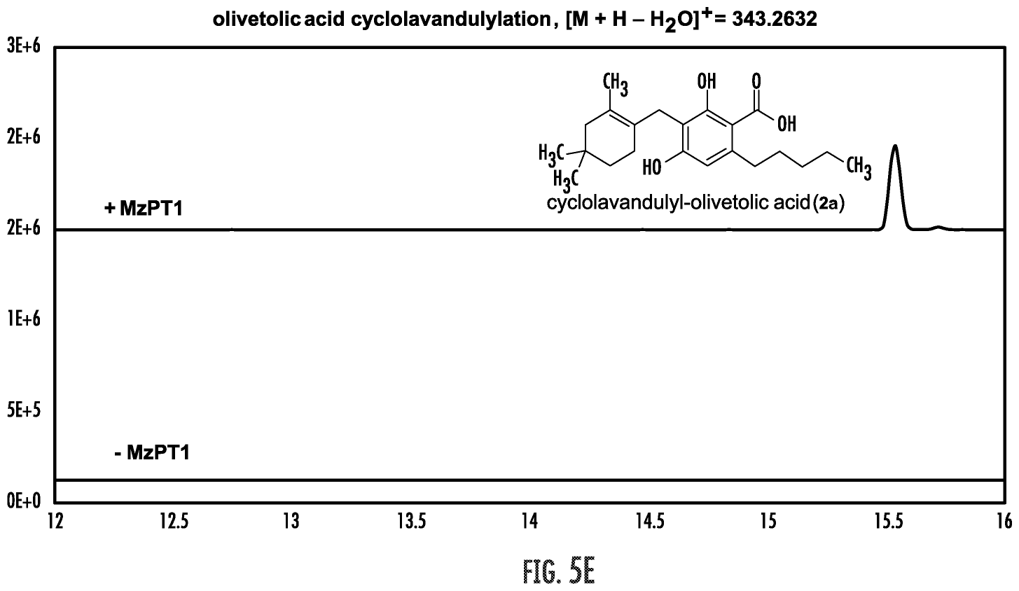

As shown by the results summarized in Table 7, the following prenyltransferases when combined in a cell-free biosynthesis reaction with CLDS, from Streptomyces sp. CL190 and DMAPP produced a cyclolavandulylated aromatic compound: NphBm31s (SEQ ID NO: 6), NphB (SEQ ID NO: 8), AbPT (SEQ ID NO: 9), NapT9 (SEQ ID NO: 14), AtaWTPT (SEQ ID NO: 17), and MzPT1 (SEQ ID NO: 18). HPLC plots of five exemplary cell-free biosynthesis reactions with and without the prenyltransferase are shown in FIG. 5A-FIG. 5E. The reactions with the added prenyltransferase yielded HPLC peaks that were further analyzed by MS and found to be consistent with the cyclolavandulylated aromatic compound product shown in the plot. Although several of the tested prenyltransferases exhibited no detectable cyclolavandulylation activity with tested aromatic compounds, it is contemplated that these enzymes may be engineered (e.g., using directed evolution techniques) and/or reaction conditions optimized so as to provide such activity.

While the foregoing disclosure of the present invention has been described in some detail by way of example and illustration for purposes of clarity and understanding, this disclosure including the examples, descriptions, and embodiments described herein are for illustrative purposes, are intended to be exemplary, and should not be construed as limiting the present disclosure. It will be clear to one skilled in the art that various modifications or changes to the examples, descriptions, and embodiments described herein can be made and are to be included within the spirit and purview of this disclosure and the appended claims. Further, one of skill in the art will recognize a number of equivalent methods and procedure to those described herein. All such equivalents are to be understood to be within the scope of the present disclosure and are covered by the appended claims.

Additional embodiments of the invention are set forth in the following claims.

The disclosures of all publications, patent applications, patents, or other documents mentioned herein are expressly incorporated by reference in their entirety for all purposes to the same extent as if each such individual publication, patent, patent application or other document were individually specifically indicated to be incorporated by reference herein in its entirety for all purposes and were set forth in its entirety herein. In case of conflict, the present specification, including specified terms, will control.

What is claimed is:

1. A compound of structural formula (Ia) or (Ib): 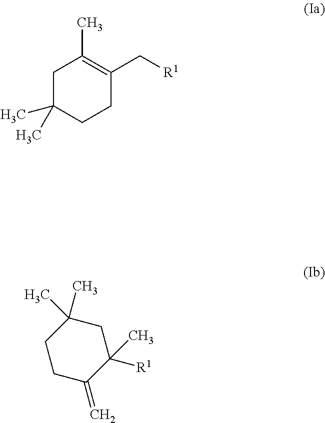
wherein, R¹ is selected from 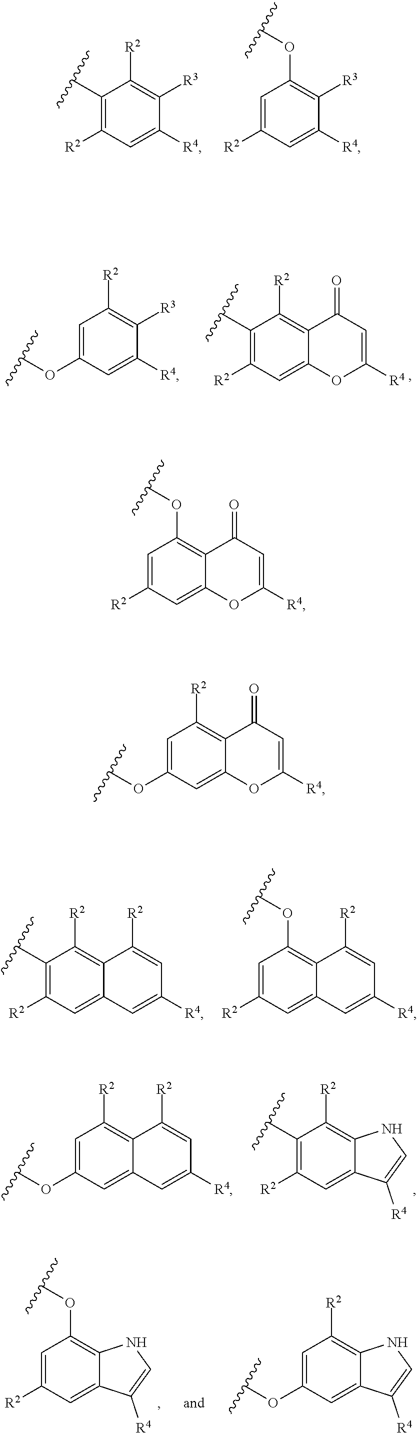
wherein,
R² is —H or —OH;
R³ is —H or —COOH; and
R⁴ is —H, —OH, linear or branched C1-C10 alkyl, linear or branched C1-C10 alkyl-amide, linear or branched C1-C10 alkyl-amine, linear or branched C1-C10 alkyl-alkylene, linear or branched C1-C10 alkyl-alkoxy, or C1-C10 alkyl-aryl, wherein any of the C1-C10 groups is optionally substituted with —OH, —OCH₃, or halogen.

2. The compound of claim 1, wherein R¹ is selected from 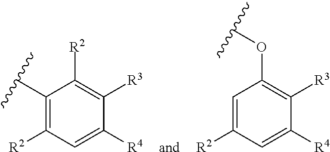
wherein,
R² is —H or —OH
R³ is —H or —COOH; and
R⁴ is linear or branched C1-C10 alkyl, linear or branched C1-C10 alkyl-amide, linear or branched C1-C10 alkyl-amine, linear or branched C1-C10 alkyl-alkylene, linear or branched C1-C10 alkyl-alkoxy, or C1-C10 alkyl-aryl, wherein any of the C1-C10 groups is optionally substituted with —OH, —OCH₃, or halogen.

3. The compound of claim 2, wherein:
(a) R² is —OH, and R³ is —COOH; and/or
(b) R⁴ is selected from CH₃, CH₂CH₃, (CH₂)₂CH₃, (CH₂)₃CH₃, (CH₂)₄CH₃, (CH₂)₅CH₃, and (CH₂)₆CH₃; optionally, wherein R⁴ is selected from (CH₂)₂CH₃, (CH₂)₄CH₃, and (CH₂)₆CH₃.

4. The compound of claim 2, wherein the compound is selected from structural formula (IIa), (IIb), (IIc), or (IId): 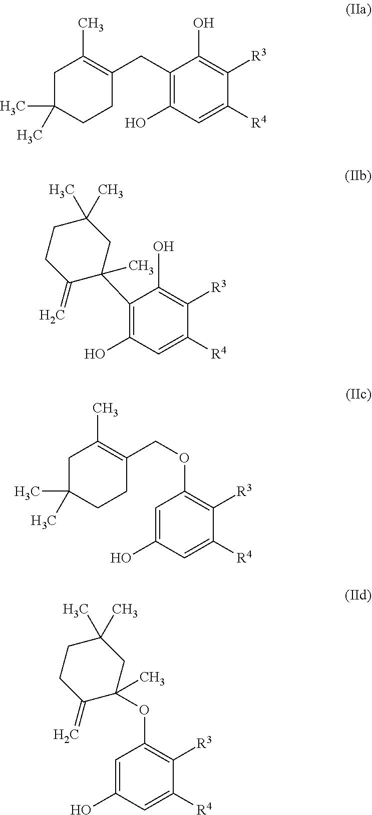
wherein,
R³ is —H or —COOH; and
R⁴ is linear or branched C1-C10 alkyl, linear or branched C1-C10 alkyl-amide, linear or branched C1-C10 alkyl-amine, linear or branched C1-C10 alkyl-alkylene, linear or branched C1-C10 alkyl-alkoxy, or C1-C10 alkyl-aryl, wherein any of the C1-C10 groups is optionally substituted with —OH, —OCH₃, or halogen.

5. The compound of claim 4, wherein R⁴ is selected from CH₃, CH₂CH₃, (CH₂)₂CH₃, (CH₂)₃CH₃, (CH₂)₄CH₃, (CH₂)₅CH₃, (CH₂)₆CH₃, and (CH₂)₇CH₃; optionally, wherein R⁴ is selected from (CH₂)₂CH₃, (CH₂)₄CH₃, and (CH₂)₆CH₃.

6. The compound of claim 5, wherein the compound is selected from compounds (2b), (2d), (2a), (2c), (2e), (2f), (2g), (2h), (2i), (2j), (2k), (2l), (2m), (2n), (2o), (2p), (2q), (2r), (2s), (2t), (2u), (2v), (2w), (2x), (2y), (2z), (2aa), (2bb), (2cc), (2dd), (2ee), (2ff), (2gg), (2hh), (2ii), (2jj), (2kk), (2ll), (2 mm), (2nn), (2oo), (2pp), (2qq), (2rr), (2ss), (2tt), (2uu), (2vv), (2ww), and (2xx):

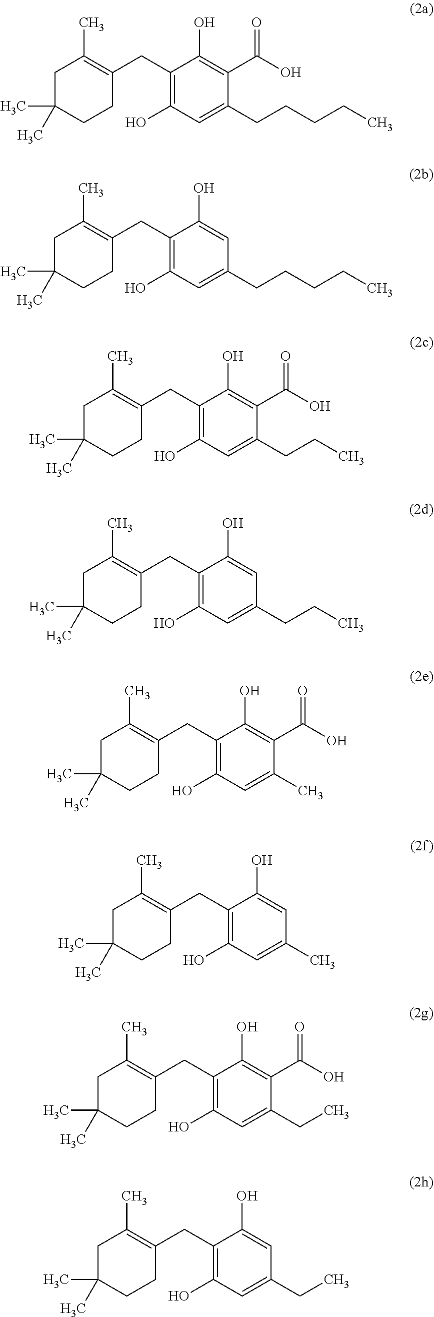

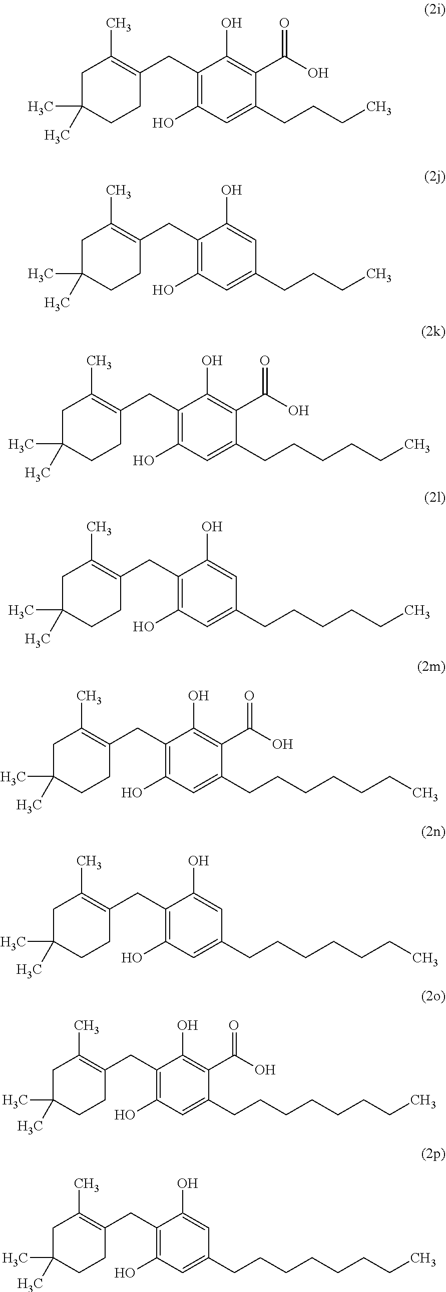

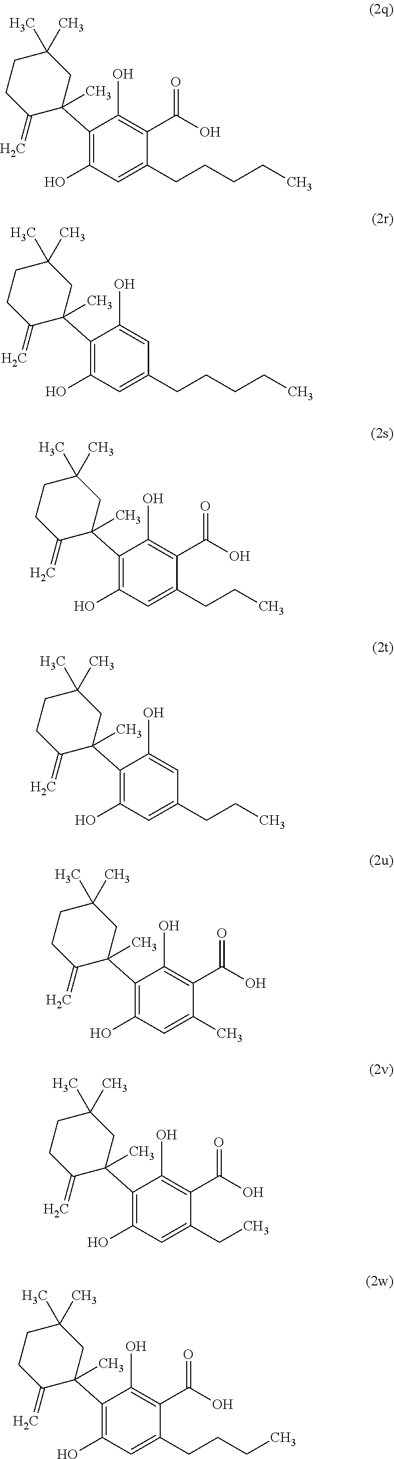

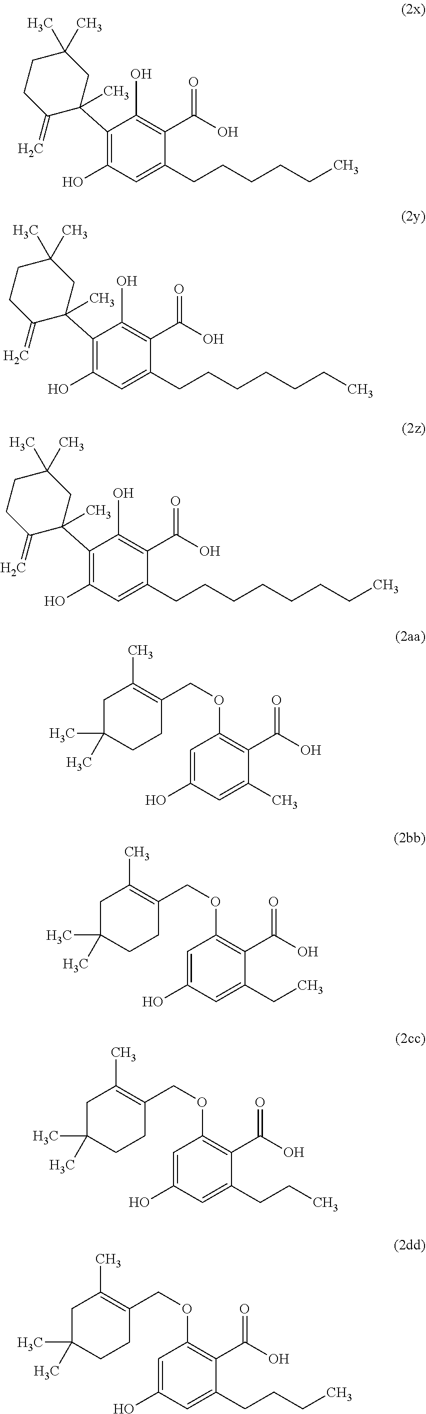

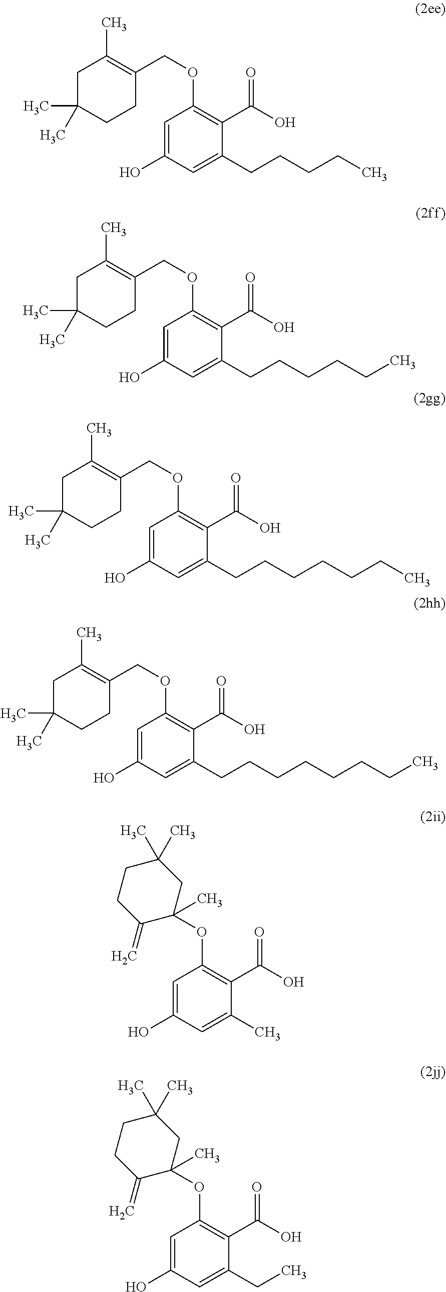

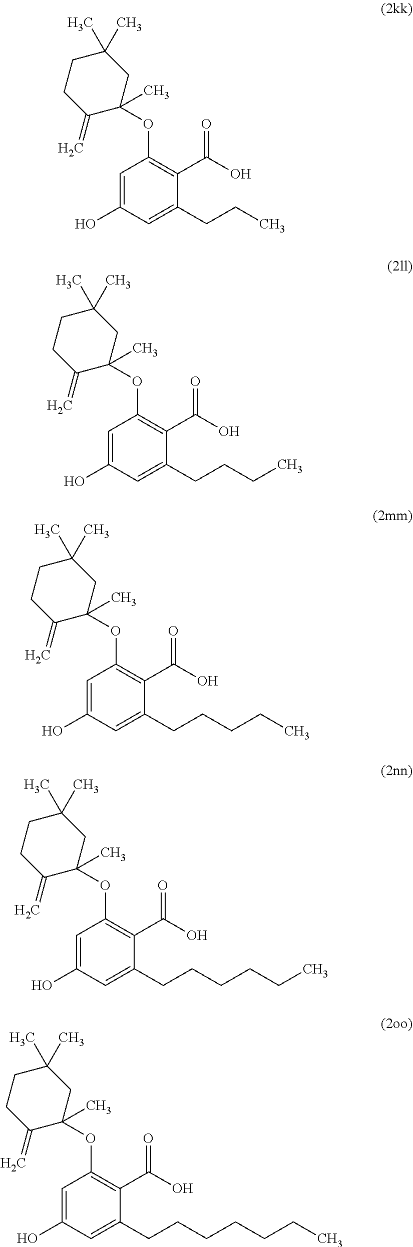 [SMILES: C=C1CCC(C)(C)CC1(C)Oc1cc(O)cc(CCC)c1C(=O)O ; C=C1CCC(C)(C)CC1(C)Oc1cc(O)cc(CCCC)c1C(=O)O ; C=C1CCC(C)(C)CC1(C)Oc1cc(O)cc(CCCCC)c1C(=O)O ; C=C1CCC(C)(C)CC1(C)Oc1cc(O)cc(CCCCCC)c1C(=O)O ; C=C1CCC(C)(C)CC1(C)Oc1cc(O)cc(CCCCCCC)c1C(=O)O]

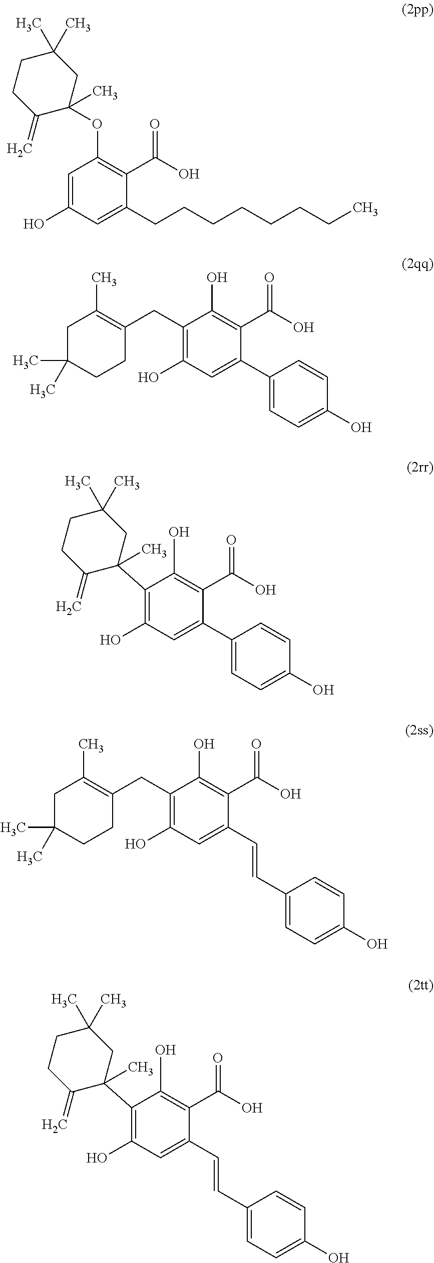

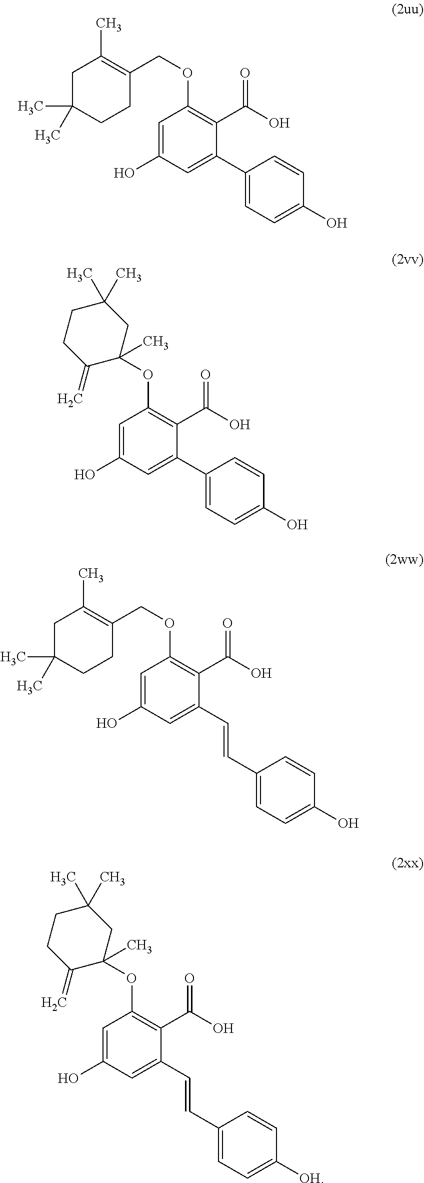 [SMILES: CC1=C(COc2cc(O)cc(-c3ccc(O)cc3)c2C(=O)O)CCC(C)(C)C1 ; C=C1CCC(C)(C)CC1(C)Oc1cc(O)cc(-c2ccc(O)cc2)c1C(=O)O ; CC1=C(COc2cc(O)cc(/C=C/c3ccc(O)cc3)c2C(=O)O)CCC(C)(C)C1 ; C=C1CCC(C)(C)CC1(C)Oc1cc(O)cc(/C=C/c2ccc(O)cc2)c1C(=O)O]

7. The compound of claim 1, wherein R¹ is selected from:

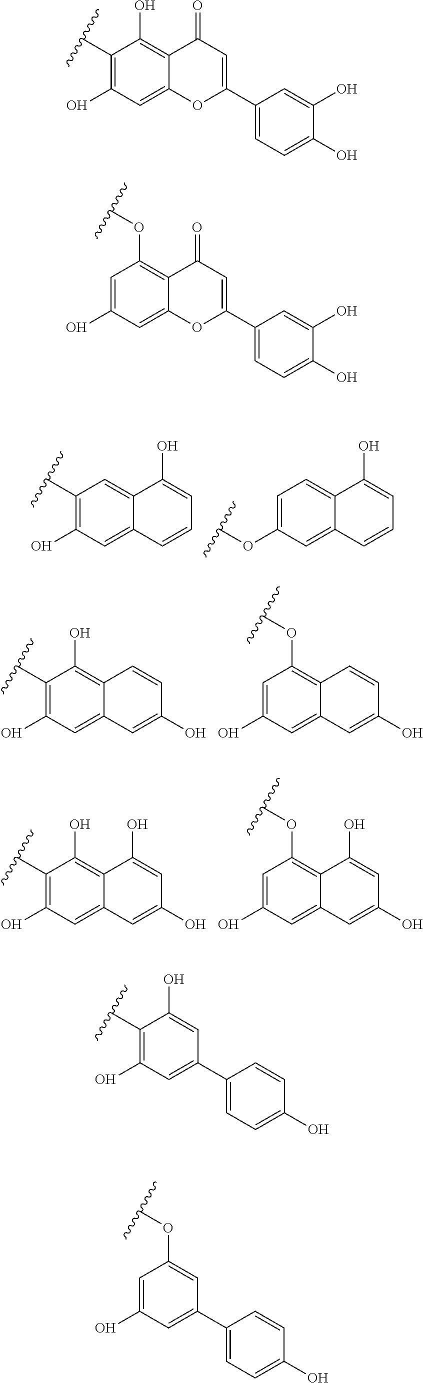

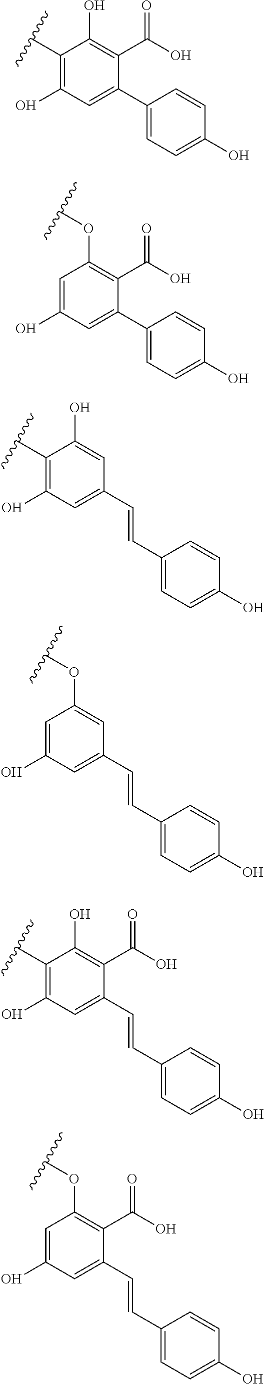 [SMILES: *c1c(O)cc(-c2ccc(O)cc2)c(C(=O)O)c1O ; *Oc1cc(O)cc(-c2ccc(O)cc2)c1C(=O)O ; *c1c(O)cc(/C=C/c2ccc(O)cc2)cc1O ; *Oc1cc(O)cc(/C=C/c2ccc(O)cc2)c1 ; *c1c(O)cc(/C=C/c2ccc(O)cc2)c(C(=O)O)c1O ; *Oc1cc(O)cc(/C=C/c2ccc(O)cc2)c1C(=O)O]

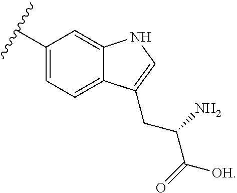 [SMILES: *c1ccc2c(C[C@H](N)C(=O)O)c[nH]c2c1]

8. A method for preparing a cyclolavandulyl-substituted aromatic compound comprising:
(a) contacting under suitable reaction conditions: a prenyitransferase, a cyclolavandulyl pyrophosphate of compound (1) 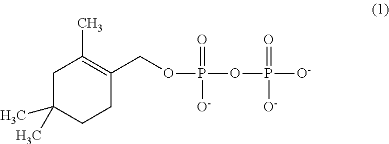 [SMILES: CC1=C(COP(=O)([O-])OP(=O)([O-])[O-])CCC(C)(C)C1]
and an aromatic compound selected from a compound of structural formulas (II), (MV), (V), and (VI) 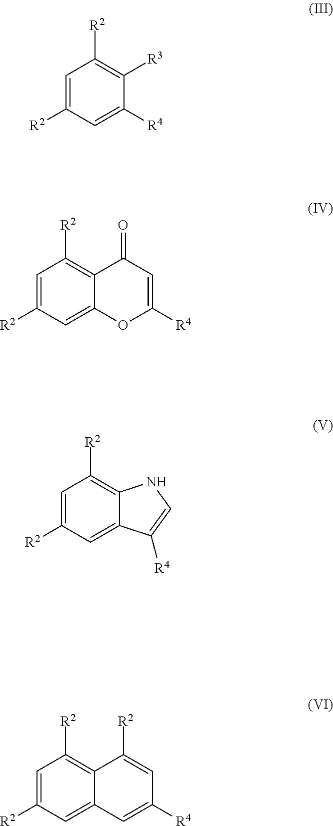
wherein,
R² is —H or —OH;
R³ is —H or —COOH; and
R⁴ is —H, —OH, linear or branched C1-C10 alkyl, linear or branched C1-C10 alkyl-amide, linear or branched C1-C10 alkyl-amine, linear or branched C1-C10 alkyl-alkylene, linear or branched C1-C10 alkyl-alkoxy, or C1-C10 alkyl-aryl, wherein any of the C1-C10 groups is optionally substituted with —OH, —OCH₃, or halogen; and
(b) recovering the cyclolavandulyl-substituted aromatic compound from the reaction mixture.

9. The method of claim 8, wherein the prenyltransferase is selected from:
(i) NphB (SEQ ID NO: 8) or a variant of NphB comprising an amino acid sequence having at least 90%, at least 95%, at least 97%, at least 98%, or at least 99% identity to any one of SEQ ID NO: 8;
(ii) NphBM31s (SEQ ID NO: 6) or a variant of NphBM31s comprising an amino acid sequence having at least 90%, at least 95%, at least 97%, at least 98%, or at least 99% identity to any one of SEQ ID NO: 6;
(iii) a prenyltransferase comprising an amino acid sequence or any one of SEQ ID NO: 9, 10, 11, 12, 13, 14, 15, 16, 17, 18, or 19; and
(iv) a variant prenyltransferase comprising an amino acid sequence having at least 90%, at least 95%, at least 97%, at least 98%, or at least 99% identity to any one of SEQ ID NO: 9, 10, 11, 12, 13, 14, 15, 16, 17, 18, or 19.

10. The method of claim 8, wherein the cyclolavandulyl-substituted aromatic compound is selected from compound (4a), (4b), (5a), (5b), (6a), (6b), (7a), (7b), (8a), and (8b):

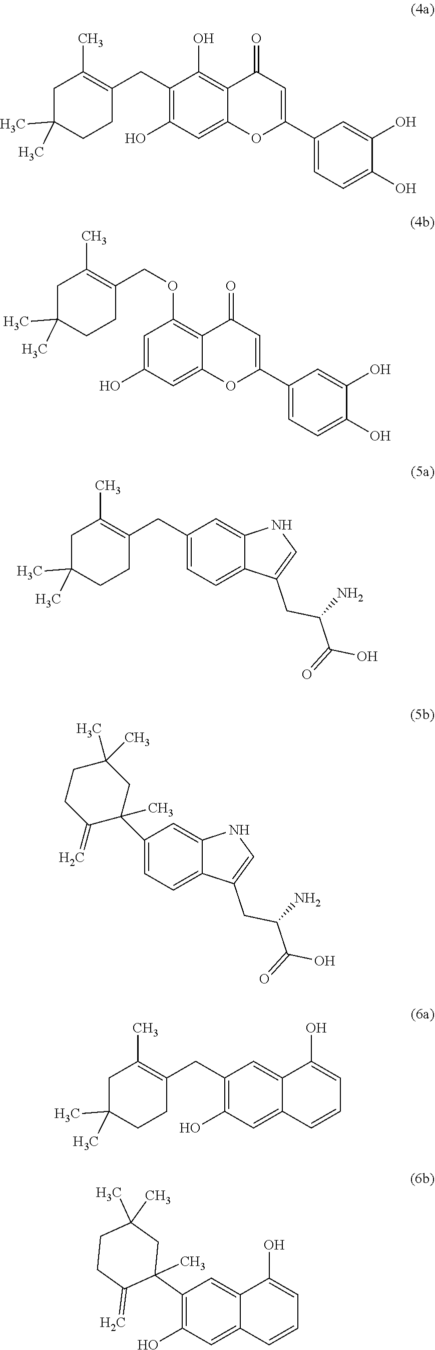 [SMILES: CC1=C(Cc2c(O)cc3oc(-c4ccc(O)c(O)c4)cc(=O)c3c2O)CCC(C)(C)C1 ; CC1=C(COc2cc(O)cc3oc(-c4ccc(O)c(O)c4)cc(=O)c23)CCC(C)(C)C1 ; CC1=C(Cc2ccc3c(C[C@H](N)C(=O)O)c[nH]c3c2)CCC(C)(C)C1 ; C=C1CCC(C)(C)CC1(C)c1ccc2c(C[C@H](N)C(=O)O)c[nH]c2c1 ; CC1=C(Cc2cc3c(O)cccc3cc2O)CCC(C)(C)C1 ; C=C1CCC(C)(C)CC1(C)c1cc2c(O)cccc2cc1O]

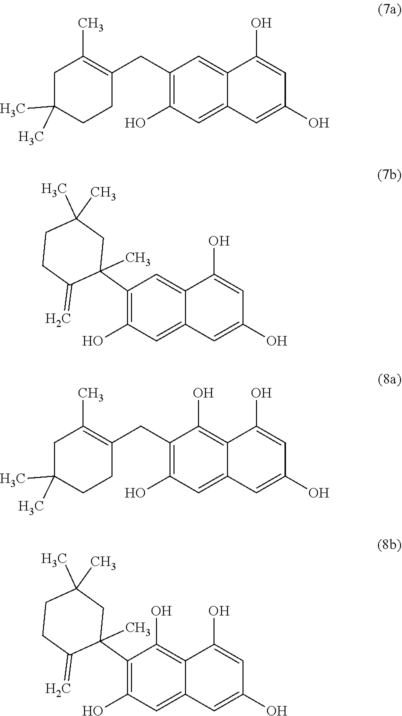 [SMILES: CC1=C(Cc2cc3c(O)cc(O)cc3cc2O)CCC(C)(C)C1 ; C=C1CCC(C)(C)CC1(C)c1cc2c(O)cc(O)cc2cc1O ; CC1=C(Cc2c(O)cc3cc(O)cc(O)c3c2O)CCC(C)(C)C1 ; C=C1CCC(C)(C)CC1(C)c1c(O)cc2cc(O)cc(O)c2c1O]

11. The method of claim 8, wherein the aromatic compound is a compound of structural formula (III): 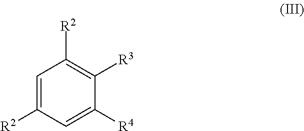
wherein,
R² is —H or —OH
R³ is —H or —COOH; and
R⁴ is linear or branched C1-C10 alkyl, linear or branched C1-C10 alkyl-amide, linear or branched C1-C10 alkyl-amine, linear or branched C1-C10 alkyl-alkylene, linear or branched C1-C10 alkyl-alkoxy, or C1-C10 alkyl-aryl, wherein any of the C1-C10 groups is optionally substituted with a —OH, —OCH₃, or a halogen.

12. The method of claim 11, wherein: (a) R² is —OH, and R³ is —COOH; and/or (b) R⁴ is selected from CH₃, CH₂CH₃, (CH₂)₂CH₃, (CH₂)₃CH₃, (CH₂)₄CH₃, (CH₂)₅CH₃, (CH₂)₆CH₃, and (CH₂)₇CH₃; optionally, wherein R⁴ is selected from (CH₂)₂CH₃, (CH₂)₄CH₃, and (CH₂)₆CH₃.

13. The method of claim 11, wherein the aromatic compound is selected from any one of compounds (3a), (3b), (3c), (3d), (3e), (3f), (3g), and (3h):

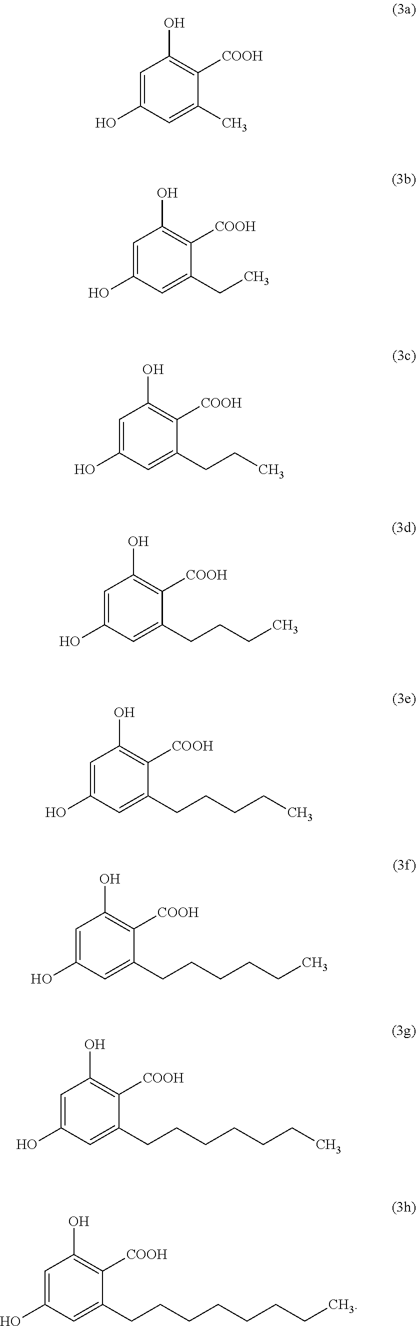

14. The method of claim 8, wherein the cyclolavandulyl-substituted aromatic compound has structural formula (IIa), (IIb), (IIc), or (IId): 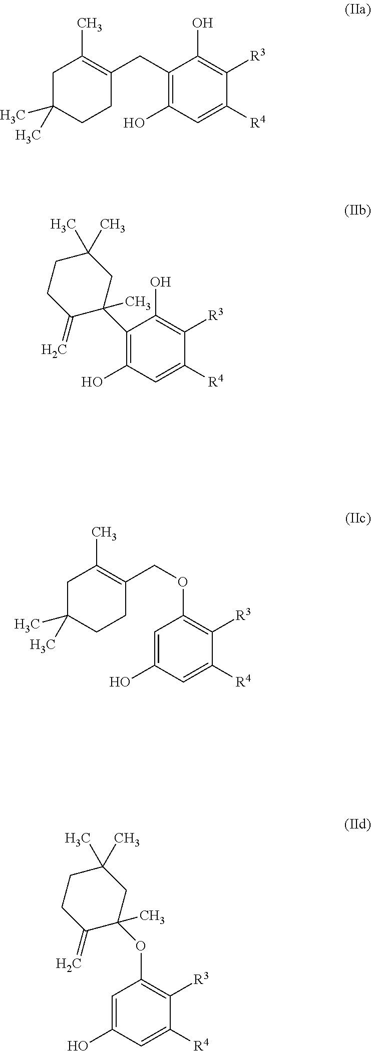
wherein,
R³ is —H or —COOH; and
R⁴ is linear or branched C1-C10 alkyl, linear or branched C1-C10 alkyl-amide, linear or branched C1-C10 alkyl-amine, linear or branched C1-C10 alkyl-alkylene, linear or branched C1-C10 alkyl-alkoxy, or C1-C10 alkyl-aryl, wherein any of the C1-C10 groups is optionally substituted with a —OH, —OCH₃, or a halogen.

15. The method of claim 14, wherein R⁴ is selected from CH₃, CH₂CH₃, (CH₂)₂CH₃, (CH₂)₃CH₃, (CH₂)₄CH₃, (CH₂)₅CH₃, (CH₂)₆CH₃, and (CH₂)₇CH₃; optionally, wherein R⁴ is selected from (CH₂)₂CH₃, (CH₂)₄CH₃, and (CH₂)₆CH₃.

16. The method of claim 14, wherein the recovered cyclolavandulyl-substituted compound is a cyclolavandulyl-substituted cannabinoid; optionally, wherein the cyclolavandulyl-substituted cannabinoid is selected from compounds (2a), (2b), (2c), (2d), (2e), (2f), (2g), (2h), (2i), (2j), (2k), (2l), (2m), (2n), (2o), (2p), (2q), (2r), (2s), (2t), (2u), (2v), (2w), (2x), (2y), (2z), (2aa), (2bb), (2cc), (2dd), (2ee), (2ff), (2gg), (2hh), (2ii), (2jj), (2kk), (2ll), (2 mm), (2nn), (2oo), (2pp), (2qq), (2rr), (2ss), (2tt), (2uu), (2vv), (2ww), and (2xx).

17. The method of claim 13, wherein the method further comprises a step of decarboxylating the recovered cyclolavandulyl-substituted compound.

18. A composition comprising a prenyltransferase, a cyclolavandulyl pyrophosphate of compound (1) 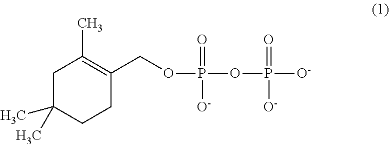 [SMILES: CC1=C(COP(=O)([O-])OP(=O)([O-])[O-])CCC(C)(C)C1]
and an aromatic compound selected from a compound of structural formula (III), (IV), (V), and (VI): 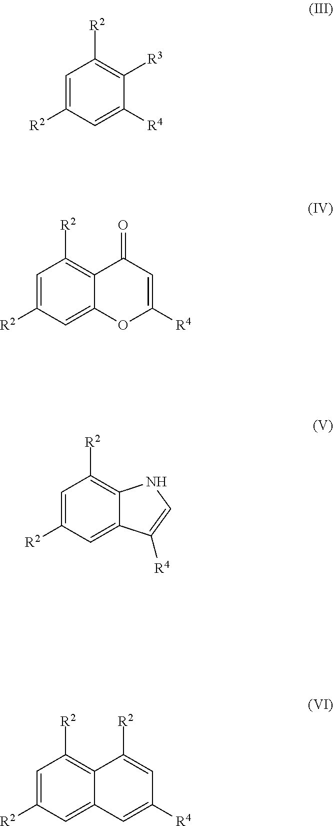
wherein,
R² is —H or —OH
R³ is —H or —COOH; and
R⁴ is —H, —OH, linear or branched C1-C10 alkyl, linear or branched C1-C10 alkyl-amide, linear or branched C1-C10 alkyl-amine, linear or branched C1-C10 alkyl-alkylene, linear or branched C1-C10 alkyl-alkoxy, or C1-C10 alkyl-aryl, wherein any of the C1-C10 groups is optionally substituted with a —OH, —OCH₃, or a halogen.

19. The composition of claim 18, wherein the prenyltransferase is selected from:
(i) NphB (SEQ ID NO: 8) or a variant of NphB comprising an amino acid sequence having at least 90%, at least 95%, at least 97%, at least 98%, or at least 99% identity to any one of SEQ ID NO: 8;
(ii) NphBM31s (SEQ ID NO: 6) or a variant of NphBM31s comprising an amino acid sequence having at least 90%, at least 95%, at least 97%, at least 98%, or at least 99% identity to any one of SEQ ID NO: 6;
(iii) a prenyltransferase comprising an amino acid sequence or any one of SEQ ID NO: 9, 10, 11, 12, 13, 14, 15, 16, 17, 18, or 19; and
(iv) a variant prenyltransferase comprising an amino acid sequence having at least 90%, at least 95%, at least 97%, at least 98%, or at least 99% identity to any one of SEQ ID NO: 9, 10, 11, 12, 13, 14, 15, 16, 17, 18, or 19.

20. The composition of claim 18, wherein the aromatic compound is a compound of structural formula (III): 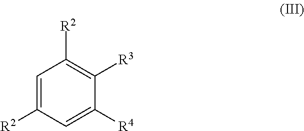
wherein,
R² is —H or —OH
R³ is —H or —COOH; and
R⁴ is linear or branched C1-C10 alkyl, linear or branched C1-C10 alkyl-amide, linear or branched C1-C10 alkyl-amine, linear or branched C1-C10 alkyl-alkylene, linear or branched C1-C10 alkyl-alkoxy, or C1-C10 alkyl-aryl, wherein any of the C1-C10 groups is optionally substituted with a —OH, —OCH₃, or a halogen.

21. The composition of claim 20, wherein: (a) R² is —OH, and R³ is —COOH; and/or (b) R⁴ is selected from CH₃, CH₂CH₃, (CH₂)₂CH₃, (CH₂)₃CH₃, (CH₂)₄CH₃, (CH₂)₅CH₃, (CH₂)₆CH₃, and (CH₂)₇CH₃; optionally, wherein R⁴ is selected from (CH₂)₂CH₃, (CH₂)₄CH₃, and (CH₂)₆CH₃.

22. The composition of claim 20, wherein the aromatic compound is selected from any one of compounds (3a), (3b), (3c), (3d), (3e), (3f), (3g), and (3h).

23. A method for preparing a cyclolavandulyl-substituted aromatic compound comprising:
(a) contacting under suitable reaction conditions cyclolavandulyl diphosphate synthase (CLDS), dimethylallyl pyrophosphate, a prenyltransferase, and an aromatic compound selected from a compound of structural formulas (III), (IV), (V), and (VI) 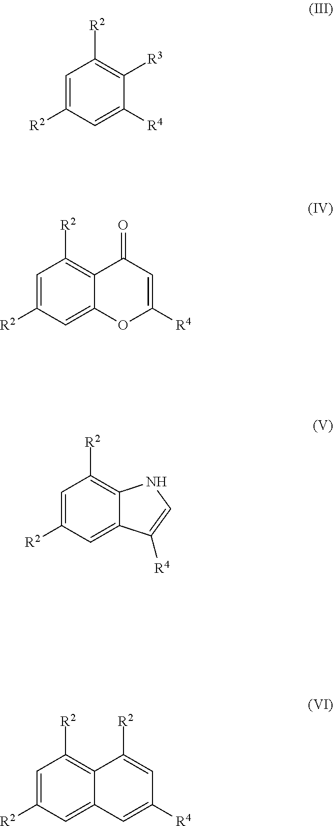
wherein,
R² is —H or —OH
R³ is —H or —COOH; and
R⁴ is —H, —OH, linear or branched C1-C10 alkyl, linear or branched C1-C10 alkyl-amide, linear or branched C1-C10 alkyl-amine, linear or branched C1-C10 alkyl-alkylene, linear or branched C1-C10 alkyl-alkoxy, or C1-C10 alkyl-aryl, wherein any of the C1-C10 groups is optionally substituted with a —OH, —OCH₃, or a halogen; and
(b) recovering the cyclolavandulyl-substituted aromatic compound from the reaction mixture.

24. The method of claim 23, wherein the cyclolavandulyl diphosphate synthase (CLDS) is a polypeptide comprises an amino acid sequence of SEQ ID NO: 2 or 4, or a variant of SEQ ID NO: 2 or 4; optionally, wherein the variant of SEQ ID NO: 2 or 4 comprises an amino acid sequence having at least 90%, at least 95%, at least 97%, at least 98%, or at least 99% identity to SEQ ID NO: 2 or 4.

25. The method of claim 23, wherein the prenyltransferase is selected from:
(i) NphB (SEQ ID NO: 8) or a variant of NphB comprising an amino acid sequence having at least 90%, at least 95%, at least 97%, at least 98%, or at least 99% identity to any one of SEQ ID NO: 8;
(ii) NphBM31s (SEQ ID NO: 6) or a variant of NphBM31s comprising an amino acid sequence having at least 90%, at least 95%, at least 97%, at least 98%, or at least 99% identity to any one of SEQ ID NO: 6;
(iii) a prenyltransferase comprising an amino acid sequence or any one of SEQ ID NO: 9, 10, 11, 12, 13, 14, 15, 16, 17, 18, or 19; and
(iv) a variant prenyltransferase comprising an amino acid sequence having at least 90%, at least 95%, at least 97%, at least 98%, or at least 99% identity to any one of SEQ ID NO: 9, 10, 11, 12, 13, 14, 15, 16, 17, 18, or 19.

26. The method of claim 23, wherein the cyclolavandulyl-substituted aromatic compound is selected from compound (4a), (4b), (5a), (5b), (6a), (6b), (7a), (7b), (8a), and (8b).

27. The method of claim 23, wherein the aromatic compound is a compound of structural formula (III): 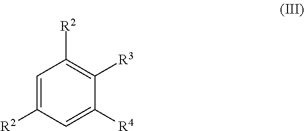
wherein,
R² is —H or —OH
R³ is —H or —COOH; and
R⁴ is linear or branched C1-C10 alkyl, linear or branched C1-C10 alkyl-amide, linear or branched C1-C10 alkyl-amine, linear or branched C1-C10 alkyl-alkylene, linear or branched C1-C10 alkyl-alkoxy, or C1-C10 alkyl-aryl, wherein any of the C1-C10 groups is optionally substituted with a —OH, —OCH₃, or a halogen.

28. The method of claim 27, wherein: (a) R² is —OH, and R³ is —COOH; and/or (b) R⁴ is selected from CH₃, CH₂CH₃, (CH₂)₂CH₃, (CH₂)₃CH₃, (CH₂)₄CH₃, (CH₂)₅CH₃, (CH₂)₆CH₃, and (CH₂)₇CH₃; optionally, wherein R⁴ is selected from (CH₂)₂CH₃, (CH₂)₄CH₃, and (CH₂)₆CH₃.

29. The method of claim 27, wherein the aromatic compound is selected from any one of compounds (3a), (3b), (3c), (3d), (3e), (3f), (3g), and (3h).

30. The method of claim 23, wherein the cyclolavandulyl-substituted aromatic compound has structural formula (II): 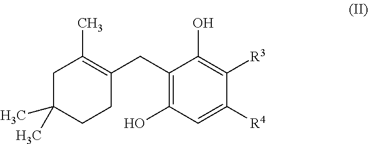
wherein,
R³ is —H or —COOH; and
R⁴ is linear or branched C1-C10 alkyl, linear or branched C1-C10 alkyl-amide, linear or branched C1-C10 alkyl-amine, linear or branched C1-C10 alkyl-alkylene, linear or branched C1-C10 alkyl-alkoxy, or C1-C10 alkyl-aryl, wherein any of the C1-C10 groups is optionally substituted with a —OH, —OCH₃, or a halogen.

31. The method of claim 30, wherein R⁴ is selected from CH₃, CH₂CH₃, (CH₂)₂CH₃, (CH₂)₃CH₃, (CH₂)₄CH₃, (CH₂)₅CH₃, (CH₂)₆CH₃, and (CH₂)₇CH₃; optionally, wherein R⁴ is selected from (CH₂)₂CH₃, (CH₂)₄CH₃, and (CH₂)₆CH₃.

32. The method of claim 30, wherein the recovered cyclolavandulyl-substituted compound is a cyclolavandulyl-substituted cannabinoid; optionally, wherein the cyclolavandulyl-substituted cannabinoid is selected from compounds (2a), (2b), (2c), (2d), (2e), (2f), (2g), (2h), (2i), (2j), (2k), (2l), (2m), (2n), (2o), (2p), (2q), (2r), (2s), (2t), (2u), (2v), (2w), (2x), (2y), (2z), (2aa), (2bb), (2cc), (2dd), (2ee), (2ff), (2gg), (2hh), (2ii), (2jj), (2kk), (2ll), (2 mm), (2nn), (2oo), (2pp), (2qq), (2rr), (2ss), (2tt), (2uu), (2vv), (2ww), and (2xx).

33. The method of claim 32, wherein the method further comprises a step of decarboxylating the recovered cyclolavandulyl-substituted compound.

34. A composition comprising a cyclolavandulyl diphosphate synthase (CLDS), dimethylallyl pyrophosphate, a prenyltransferase, and an aromatic compound selected from a compound of structural formulas (III), (IV), (V), and (VI) 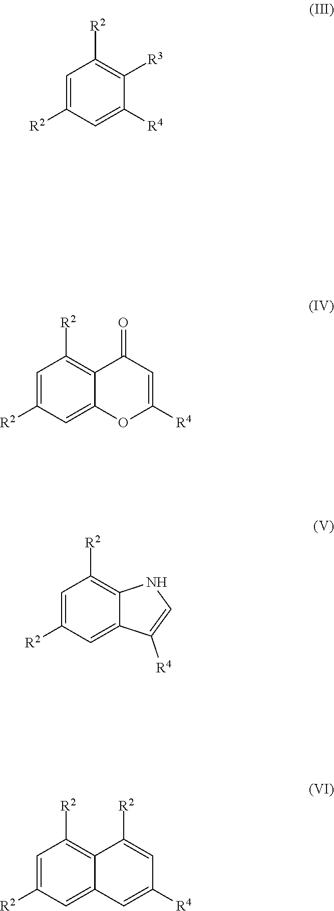
wherein,
R² is —H or —OH
R³ is —H or —COOH; and
R⁴ is —H, —OH, linear or branched C1-C10 alkyl, linear or branched C1-C10 alkyl-amide, linear or branched C1-C10 alkyl-amine, linear or branched C1-C10 alkyl-alkylene, linear or branched C1-C10 alkyl-alkoxy, or C1-C10 alkyl-aryl, wherein any of the C1-C10 groups is optionally substituted with a —OH, —OCH₃, or a halogen.

35. The composition of claim 34, wherein the cyclolavandulyl diphosphate synthase (CLDS) is a polypeptide comprises an amino acid sequence of SEQ ID NO: 2 or 4, or a variant of SEQ ID NO: 2 or 4; optionally, wherein the variant of SEQ ID NO: 2 or 4 comprises an amino acid sequence having at least 90%, at least 95%, at least 97%, at least 98%, or at least 99% identity to SEQ ID NO: 2 or 4.

36. The composition of claim 34, wherein the prenyltransferase is selected from:
(i) NphB (SEQ ID NO: 8) or a variant of NphB comprising an amino acid sequence having at least 90%, at least 95%, at least 97%, at least 98%, or at least 99% identity to any one of SEQ ID NO: 8;
(ii) NphBM31s (SEQ ID NO: 6) or a variant of NphBM31s comprising an amino acid sequence having at least 90%, at least 95%, at least 97%, at least 98%, or at least 99% identity to any one of SEQ ID NO: 6;
(iii) a prenyltransferase comprising an amino acid sequence or any one of SEQ ID NO: 9, 10, 11, 12, 13, 14, 15, 16, 17, 18, or 19; and
(iv) a variant prenyltransferase comprising an amino acid sequence having at least 90%, at least 95%, at least 97%, at least 98%, or at least 99% identity to any one of SEQ ID NO: 9, 10, 11, 12, 13, 14, 15, 16, 17, 18, or 19.

37. The composition of claim 34, wherein the aromatic compound is a compound of structural formula (III): 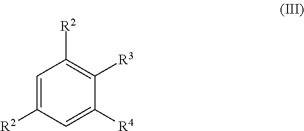
wherein,
R² is —H or —OH
R³ is —H or —COOH; and
R⁴ is linear or branched C1-C10 alkyl, linear or branched C1-C10 alkyl-amide, linear or branched C1-C10 alkyl-amine, linear or branched C1-C10 alkyl-alkylene, linear or branched C1-C10 alkyl-alkoxy, or C1-C10 alkyl-aryl, wherein any of the C1-C10 groups is optionally substituted with a —OH, —OCH₃, or a halogen.

38. The composition of claim 37, wherein: (a) R² is —OH, and R³ is —COOH; and/or (b) R⁴ is selected from CH₃, CH₂CH₃, (CH₂)₂CH₃, (CH₂)₃CH₃, (CH₂)₄CH₃, (CH₂)₅CH₃, (CH₂)₆CH₃, and (CH₂)₇CH₃; optionally, wherein R⁴ is selected from (CH₂)₂CH₃, (CH₂)₄CH₃, and (CH₂)₆CH₃.

39. The composition of claim 37, wherein the aromatic compound is selected from any one of compounds (3a), (3b), (3c), (3d), (3e), (3f), (3g), and (3h).

40. The composition of claim 37, wherein the aromatic compound is a cannabinoid precursor compound selected from divarinic acid (DA), olivetolic acid (OA), and phorolic acid (PA).


SEQUENCE LISTING (19 sequences)

SEQ ID NO: 1 - DNA, 654 bp, synthetic construct
  misc_feature 1..654: note = Synthetic polymer
  source 1..654: mol_type = other DNA
atgactacattaatgctgctgccggacggaatgcgtcgctggtcggaaaaaaatggcgtaagtctggatgatggttatgcggcaatgggtgataaaattatcgagtttatgggttgggcaaaggaggagggcgtaaagactctgtacatcactgcgtcaagcgcggcgaaccatgggcgcccggaggcagccgtgaacacctttatggaagcgttcaccgaagttatccgtcgttgccacagccagtttaaattcgacttctctggttcccttgacttagtctctgaggactatctgacggagttatcggcgttacgtgataagtcagactccgagtcggatttcacactgcattatattctggggatgagtttgtcccatgaagtggtaggaatcttcaataaacttaatggcaaaatccccgaaatgactgaagaaattctggctgagaatgcatatgtgcccacgcaggtggattatattattcgcactggcggcgctatccggatgtcatcattctttccactgatgtcgccgtatgctgaattacacttctctcctgttttattcccggatacaacacgcgctgatttcgatgcggctttgaaagatctgcgtgcccgtgatcgtcgctttggtggatacccggcgtga

SEQ ID NO: 2 - protein, 217 aa, Streptomyces sp.
MTTLMLLPDGMRRWSEKNGVSLDDGYAAMGDKIIEFMGWAKEEGVKTLYITASSAANHGRPEAAVNTFMEAFTEVIRRCHSQFKFDFSGSLDLVSEDYLTELSALRDKSDSESDFTLHYILGMSLSHEVVGIFNKLNGKIPEMTEEILAENAYVPTQVDYIIRTGGAIRMSSFFPLMSPYAELHFSPVLFPDTTRADFDAALKDLRARDRRFGGYPA

SEQ ID NO: 3 - DNA, 714 bp, synthetic construct
  misc_feature 1..714: note = Synthetic polymer
  source 1..714: mol_type = other DNA
atgggcagcagccatcatcatcatcatcacagcagcggcctggtgccgcgcggcagccatatgactacattaatgctgctgccggacggaatgcgtcgctggtcggaaaaaaatggcgtaagtctggatgatggttatgcggcaatgggtgataaaattatcgagtttatgggttgggcaaaggaggagggcgtaaagactctgtacatcactgcgtcaagcgcggcgaaccatgggcgcccggaggcagccgtgaacacctttatggaagcgttcaccgaagttatccgtcgttgccacagccagtttaaattcgacttctctggttcccttgacttagtctctgaggactatctgacggagttatcggcgttacgtgataagtcagactccgagtcggatttcacactgcattatattctggggatgagtttgtcccatgaagtggtaggaatcttcaataaacttaatggcaaaatccccgaaatgactgaagaaattctggctgagaatgcatatgtgcccacgcaggtggattatattattcgcactggcggcgctatccggatgtcatcattctttccactgatgtcgccgtatgctgaattacacttctctcctgttttattcccggatacaacacgcgctgatttcgatgcggctttgaaagatctgcgtgcccgtgatcgtcgctttggtggatacccggcgtga

SEQ ID NO: 4 - protein, 237 aa, synthetic construct
  REGION 1..237: note = Synthetic polymer
MGSSHHHHHHSSGLVPRGSHMTTLMLLPDGMRRWSEKNGVSLDDGYAAMGDKIIEFMGWAKEEGVKTLYITASSAANHGRPEAAVNTFMEAFTEVIRRCHSQFKFDFSGSLDLVSEDYLTELSALRDKSDSESDFTLHYILGMSLSHEVVGIFNKLNGKIPEMTEEILAENAYVPTQVDYIIRTGGAIRMSSFFPLMSPYAELHFSPVLFPDTTRADFDAALKDLRARDRRFGGYPA

SEQ ID NO: 5 - DNA, 924 bp, synthetic construct
  misc_feature 1..924: note = Synthetic polymer
  source 1..924: mol_type = other DNA
atgtcggaagctgccgatgtagaacgtgtctacgccgccatcgaagaagccgcaggtttgttgggggtcgcatgcgcacgcgataagatttggcccttgctgtcaacattccaggataccttggttgagggtggaagcgtagttgtttttagcatggcctcggggcgtcactcaacggagctggacttctcaatttccgtcccgcctagtcatggcgatccgtacgcgattgtggtggaaaagggcttgttcccggcaactggacatccagttgatgaccttctggcggacattcagaagcatcttcccgtatctatgtttgcgattgacggggaagttaccggggggttcaaaaaaacttatgcgttcttcccgaccgataacatgcccggtgtcgcggaactggcggccatcccatcgatgcctcctgcagtcgctgaaaatgctgaactgttcgcgcgttatggcctggacaaggtacaaatgacctcgatggattataaaaaacgtcaagtgaacctgtatttctccgaactgtcggctcagacgctggaggctgaatcagtacttgctttagtgcgtgaactgggtcttcatgtcccaaacgagctgggtctgaaattttgcaaacgctccttctcagtatacccaacattaaactgggacacctcgaagattgaccgcctttgcttctctgtaatcagtacagatccgacacttgtacctagctcagacgagggagacattgaaaaatttcacaattacgctacaaaggccccctatgcatatgttggagaaaagcgtacacttgtttacggcttgactttatctcccaaagaggagtattataaattgggtgccgtttaccacattactgacgtacaacgcaaacttttgaaggcgttcgacagccttgaggattaa

SEQ ID NO: 6 - protein, 307 aa, synthetic construct
  REGION 1..307: note = Synthetic polymer
MSEAADVERVYAAIEEAAGLLGVACARDKIWPLLSTFQDTLVEGGSVVVFSMASGRHSTELDFSISVPPSHGDPYAIVVEKGLFPATGHPVDDLLADIQKHLPVSMFAIDGEVTGGFKKTYAFFPTDNMPGVAELAAIPSMPPAVAENAELFARYGLDKVQMTSMDYKKRQVNLYFSELSAQTLEAESVLALVRELGLHVPNELGLKFCKRSFSVYPTLNWDTSKIDRLCFSVISTDPTLVPSSDEGDIEKFHNYATKAPYAYVGEKRTLVYGLTLSPKEEYYKLGAVYHITDVQRKLLKAFDSLED

SEQ ID NO: 7 - DNA, 924 bp, synthetic construct
  misc_feature 1..924: note = Synthetic polymer
  source 1..924: mol_type = other DNA
atgtccgaggccgcagatgtagaacgcgtttacgcagctatggaggaagccgcagggctgctgggggtggcgtgtgctcgcgacaaaatctatccccttctttcgacgtttcaggacacactggtagaaggaggctccgttgttgtgtttagcatggcttccggtcgtcacagtacagagcttgatttctcaatttccgtacccacgtcgcatggggacccctatgccactgttgtggagaaggggttgtttccagcgacagggcaccctgtcgatgacctgcttgctgatactcaaaaacatttacccgtcagcatgtttgctatcgatggcgaagtcaccggcggcttcaagaagacatatgcttttttcccaacagataatatgcctggcgtagcagagttgtcggctatcccgagtatgccgcccgcagtggccgagaacgctgagctgttcgctcgttatggccttgataaggttcaaatgacaagtatggattacaagaagcgtcaggtaaacctgtacttcagcgagctttcagctcaaaccctggaagccgagtccgtattagcccttgtacgtgagttgggcttacacgtaccaaacgagcttggactgaaattctgtaaacgctcgtttagtgtttaccctacattaaattgggagactggcaagattgatcgtttatgcttcgcagtgatctccaacgatccgactctggtgccgagctctgatgaaggagatattgaaaaatttcataattatgctacaaaggccccgtatgcatatgttggcgaaaagcgcactcttgtctatggcctgactttaagtcccaaggaggagtactataaactgggagcatattaccatatcaccgacgttcaacgcggacttttgaaagcgtttgactcattggaggattaa

SEQ ID NO: 8 - protein, 307 aa, Streptomyces sp. [sequence omitted]

SEQ ID NO: 9 - protein, 299 aa, Actinomyces sp.
MSGKANIQRVYSAVEEAADLLDIACSREQMWPTLTAFEDVLTDGAIVFNMVTSGGRIGDLSFDFTLPPSAGDPYELALAHGLVDETDHPVRALFSDIQARFPLQAYGVDHGITSGFHKTYAFFPLGDLQSVAELADIPSMPSGLSEHVTSFAAYGLDDKVSAIAIDYARRTWNVYFNGLSAPHVQRTAVLSMLSEFGLPEPSAQLLEFIETSSALYPTFGWDSSKIERISFSTRTTDPVALPARIEPKLEKFARSAPHVYEGERVLVYAGALSPSEEYYKLATYYQMTAAAHDRVRSAN

SEQ ID NO: 10 - protein, 307 aa, Streptomyces sp.
MPGTDDVAVDVASVYSAIEKSAGLLDVTAAREVVWPVLTAFEDVLEQAVIAFRVATNARHEGDFDVRFTVPEEVDPYAVALSRSLIAKTDHPVGSLLSDIQQLCSVDTYGVDLGVKSGFKKVWVYFPAGEHETLARLTGLTSMPGSLAGNVDFFTRYGLADKVDVIGIDYRSRTMNVYFAAPSECFERETVLAMHRDIGLPSPSEQMFKFCENSFGLYTTLNWDTMEIERISYGVKTENPMTFFARLGTKVEHFVKNVPYGVDTQKMVYAAVTSSGEEYYKLQSYYRWRSVSRLNAAYIAARDKEST

SEQ ID NO: 11 - protein, 299 aa, Streptomyces sp.
MSEASELADIYSAIEETAQLLDVPCSRDKVQPALAAFGDGLADAHIVFSMATGERYKGELAFDFTVPTAAGDPYAIALANGLVEETDHPIRTLFSDVQKRCPVDSYGVDYGIVGGFKKTYISFPLGDLQRLSALVGIPSMPRALAEHADFFASHGLDDKVSAIAIDYAHRTWNVYFSGIPAEVKEPQTLQSVLQKFGLPEPSEQLMDFIRTSFAMYTTFGWDSTKAERICFSARSSDPTALPAQFEPQIGKFAKSAPYTYTGERVLTYAGALSPSEEFYKLATFYQKTSKLSDRVRPAT

SEQ ID NO: 12 - protein, 305 aa, Streptomyces sp.
MSGAADVERVYAAMEEAAGLLDVSCAREKIYPLLTVFQDTLTDGVVVFSMASGRRSTELDFSISVPVSQGDPYATVVKEGLFQATGSPVDELLADTVAHLPVSMFAIDGEVTGGFKKTYAFFPTDDMPGVAQLAAIPSMPASVAENAELFARYGLDKVQMTSMDYKKRQVNLYFSDLKQEYLQPESVVALARELGLRVPGELGLEFCKRSFAVYPTLNWDTGKIDRLCFAAISTDPTLVPSEDERDIEMFRNYATKAPYAYVGEKRTLVYGLTLSSTEEYYKLGAYYHITDIQRQLLKAFDALED

SEQ ID NO: 13 - protein, 297 aa, Streptomyces aculeolatus
MEGLYAAIEEASGLLDVAPSRDKVWPILSAYELDKVVVAFRVTTRGSKDLDCRFTALPADVNPYRYAVSKGIAEATDHPVGTLLDDVQANIPVTAHGVDFGVVEGFRKTWTFLPGNDLQKLSKVAALPSMPPSLAENLDFYARYGLDDKNSMIGIDYPSRTVNVYFLQFPDETREPETVRAMLRDLGLPEPSEQMLTLAKQAVGIYTTLTWDSPKIQRITFATLVPDAEALPGRIAVEPSVEKFARNVPHTYPGPVQGLYNVASYSGGEYFKLQTYHQLAEGSLEARVLLGAAGAGS

SEQ ID NO: 14 - protein, 306 aa, Streptomyces sp.
MSGATGAEKLYSAIEESARMVDAPFSRDKVWPTLSAFEGGFSDAGGVILSLQAGTHVPEMEYSAQVSPGISDPYARALASGILTETDHPVSTVLAEIVSLAPTSEHYIDCGIVGGFKKIYANFPHDQQTVAALAGLPSMPRAVGGNAEFFARHGLDRVALIGVDYVNKTINLYFQVSAATAGNLDQKTVSAMLHETGMSEPSDAMVAYACQAYRIYTTLSWDAQEILRIAFAPKPRRGIDPADLPARLEPRIEKFLRATPHKYPGALINATAAKWSPEREVLDLAAHYQVSAVQMKAIEAEEGQAS

SEQ ID NO: 15 - protein, 305 aa, Streptomyces sp.
MSGETDAEELCSAIEETARLVGAPCSREKIWPILTEYRSGFAEGGVVFSAQAGENHAGELDYGLAVPPRIDDPYAHALAHGFVTETDHPVAALLSDIRERCAVTEHFADCGVVGGFRKLYAHFPRDLQKVSEIADIPSMPRAVAENVGLFTRYGLENVVMVGVNYKNKTVSLYFQFTAEGRPRPSAIRSMLGDIGMTEPNERMLDFASNSFRANITLSWDSPKILRVAFAPPPGAGLNLSTVPIPTGPHLEDFVTRAPRAYDGERMNLFAVKWTQDKEFLEVCSYYQLPAGYEPIRQMEIHKEQG

SEQ ID NO: 16 - protein, 305 aa, Streptomyces sp.
MSGAADVERVYSAMEEAARLLDITVSREKVRPALEAYHEVLADAVVVFSMASGRYATELDFSISVPAEAGDPYRVALAKGLTPRTDHPVGRLLADTQEHCPVSMFAFDGEITGGFKKTYAFFPTNDLQSASKLAEIPSMPDSVKENADLFARYGLDKVQMTSIDYKKKAVNLYFSEMSPDILGPDTVRSMLRDMGLKETGETGLTFARRSFSVYPTLNWETGRIERLCFAVISRDPTLAPAERAEDLAKFSKYANNAPYAYAGEARTLVYGLTLTPREEYYKLGSYYQISDIQRKLLKAFDSLND

SEQ ID NO: 17 - protein, 424 aa, Aspergillus terreus
MLPPSDSKDPRPWQILSQALGFPNYDQELWWQNTAETLNRVLEQCDYSVHLQYKYLAFYHKYILPSLGPFRRPGVEPEYISGLSHGGHPLEISVKIDKSKTICRLGLQAIGPLAGTARDPLNSFGDRELLKNLATLLPHVDLRLFDHFNAQVGLDRAQCAVATTKLIKESHNIVCTSLDLKDGEVIPKVYFSTIPKGLVTETPLFDLTFAAIEQMEVYHKDAPLRTALSSLKDFLRPRVPTDASITPPLTGLIGVDCIDPMLSRLKVYLATFRMDLSLIRDYWTLGGLLTDAGTMKGLEMVETLAKTLKLGDEACETLDAERLPFGINYAMKPGTAELAPPQIYFPLLGINDGFIADALVEFFQYMGWEDQANRYKDELKAKFPNVDISQTKNVHRWLGVAYSETKGPSMNIYYDVVAGNVARV

SEQ ID NO: 18 - protein, 305 aa, Streptomyces sp.
MSRTAEVERVYSAIEEAAGLLSAVCSRDNVWPVLDAFGDELAEAMIVFGGQAGDRHAGELDYSFTVPSSIGNPYPHALSRGLVTETDHPVGSVLSDIQGRWPIKEHFVDAGVVGGFKKLYAHFPDNMQKVSDIADIPSMPRAVAENAALFARYGLDQVAMIGVDYRHKTVSVYFHFGPDGRPEPSTIRALLSDLGMPEPDEPTLEFAHKSLRVNITLGWETGDVIRVAFAPPPVRGLDPSALPGQLTPRMEEFARVSPRAYAGGRVNMYAVKWMRGEEFHEVCTYYQLTPWYEKILTDAPKEQQG

SEQ ID NO: 19 - protein, 301 aa, Streptomyces sp.
MSGTAVEKLYSAIEETAGLLDVTCSRDKVWPVLASFEDAIPQAAILFRVATDKRHAGELNLHFMMLPGDVDPYALALSNDLMAKTEHPVGSLLSDIAERFPVESYGIDFGVVKGFQKTWSCFPGNSMQSLSKLAEVPSVPSSLAGNLDFFSRYGLDENVTLVGIDFEHRTMNVYFGEVAELLEPDAVKSMLRDLEMPEPSEQLLNFAKQSFGFYATLSWDSPRIERFCYAAIAPDPMALLDQVEPKIEHFLKTVPYGVDDPKAVYAATSPNDGEFYKIQSYYQWRPRLVNHMHTSVSDSGE